(19) United States
(12) Patent Application Publication
(10) Pub. No.: US 2026/0174679 A1
(43) Pub. Date: Jun. 25, 2026

(54) METHODS OF SELECTING CANCER PATIENTS AMENDABLE TO TREATMENT WITH IMMUNE CHECKPOINT INHIBITOR

(71) Applicant: The Regents of the University of California, Oakland, CA (US)

(72) Inventors: Bridget KEENAN, San Francisco, CA (US); Lawrence FONG, San Francisco, CA (US); Chun YE, San Francisco, CA (US)

(21) Appl. No.: 19/129,254

(22) PCT Filed: Nov. 17, 2023
(86) PCT No.: PCT/US2023/080272

§ 371 Date: May 12, 2025

Related U.S. Application Data
(60) Provisional application No. 63/384,362, filed on Nov. 18, 2022.

(51) Int. Cl. A61K 9/00; A61K 40/11; A61K 45/06

(52) U.S. Cl. CPC A61K 9/0019; A61K 40/11; A61K 45/06

(57) ABSTRACT

The present disclosure describes methods for selecting a cancer subject amendable to a treatment with one or more immune checkpoint inhibitors. The present disclosure also describes methods for treating cancer in a subject having or suspected of having cancer with one or more immune checkpoint inhibitors by specifically selecting a subject who will respond to the treatment with the one or more immune checkpoint inhibitors.

### RELATED APPLICATION

This application claims priority to U.S. Provisional Application Ser. No. 63/384,362, filed on Nov. 18, 2022, which is incorporated herein by reference in its entirety for all purpose.

### STATEMENT REGARDING FEDERALLY SPONSORED RESEARCH OR DEVELOPMENT

This invention was made with government support under grant Nos. U01 CA233100, R35 CA253175 awarded by the National Institutes of Health. The government has certain rights in the invention.

### BACKGROUND

While immune checkpoint inhibition can lead to dramatic clinical responses in specific cancers, many solid malignancies are “cold tumors,” insensitive to this treatment approach. Advanced biliary tract cancers (BTCs), a complex family of epithelial cancers including intrahepatic and extrahepatic cholangiocarcinoma and gallbladder cancer, represent such a tumor with a poor prognosis and particularly low responses rates under 10% to immune checkpoint inhibition. BTCs are characterized by an immunosuppressive microenvironment, desmoplastic stroma, and few effector T cells. The mechanisms that underlie primary resistance to immune checkpoint inhibition are not fully elucidated, leading to inability to determine whether a patient is amendable to a treatment with an immune checkpoint inhibitor (CPI) in a clinical setting. Therefore, there is a critical need to be able to determine whether a cancer patient is or will be amendable to a CPI treatment at the early stage of or prior to administering the CPI to the patient.

### SUMMARY

Recognized herein is a need to determine whether a patient will respond to a treatment with an immune checkpoint inhibitor (CPI). The present disclosure describes methods for selecting a subject amendable to a treatment with an immune checkpoint inhibitor. The present disclosure also describes methods for treating cancer in a subject having or suspected of having cancer with an immune checkpoint inhibitor by specifically selecting the subject who will respond to the CPI treatment.

Provided herein is a method of selecting a subject amendable to treatment with one or more immune checkpoint inhibitors from one or more subjects having or suspected of having cancer, the method including: (a) isolating a test population of peripheral blood mononuclear cells (PBMCs) from the subject and one or more reference populations of PBMCs from each subject of the one or more subjects, wherein each reference population can be isolated independently from each subject of the one or more subjects; (b) quantifying frequency of CD14_{CTX} cells in the test population; (c) quantifying frequency of CD14_{CTX} cells in the each reference population independently, and averaging the frequency CD14_{CTX} cells in the one or more reference populations; (d) comparing the frequency of CD14_{CTX} cells in the test population to the average frequency of CD14_{CTX} in the one or more reference population; and (e) selecting the subject as the subject amendable to the treatment if the frequency of CD14_{CTX} cells in the test population is lower than the average frequency of CD14_{CTX} cells in the one or more reference populations.

Provided herein is a method of treating cancer in a subject having or suspected of having the cancer with one or more immune checkpoint inhibitors, the method including: (a) isolating a test population of PBMCs from the subject and one or more reference populations of PBMCs from one or more subjects having or suspected of having the cancer, wherein each reference population can be isolated independently from each subject of the one or more subjects; (b) quantifying frequency of CD14_{CTX} cells in the test population; (c) quantifying frequency of CD14_{CTX} cells in the each reference population independently, and averaging the frequency of CD14_{CTX} cells in the one or more reference populations; (d) comparing the frequency of CD14_{CTX} cells in the test population to the average frequency of CD14_{CTX} in the one or more reference populations; and (e) administering a therapeutically effective amount of the one or more immune checkpoint inhibitors to the subject if the frequency of CD14_{CTX} cells in the test population is lower than the average frequency of CD14_{CTX} cells in the one or more reference populations, thereby treating the cancer in the subject.

In some embodiments, the lower frequency of CD14_{CTX} cells can lead to higher disease-free survival (DFS). In some embodiments, the frequency of CD14_{CTX} cells can be determined by sequencing PBMCs. In some embodiments, a cell surface marker of the CD14_{CTX} cells can be selected from the group consisting of T cell immunoglobulin and mucin domain-containing protein 3 (Tim3), CD29 (integrin β1), CD14, CD63, and CD68. In some embodiments, the cell surface marker can be Tim3. In some embodiments, the cell surface marker can be CD29.

Provided herein is a method of selecting a subject amendable to treatment with one or more immune checkpoint inhibitors from one or more subjects having or suspected of having cancer, the method including: (a) isolating a test population of peripheral blood mononuclear cells (PBMCs) from the subject and one or more reference populations of PBMCs from each subject of the one or more subjects, wherein each reference population can be isolated independently from each subject of the one or more subjects; (b) quantifying frequency of CD4_{SOCS3} cells in the test population; (c) quantifying frequency of CD4_{SOCS3} cells in the each reference population independently, and averaging the frequency CD4_{SOCS3} cells in the one or more reference populations; (d) comparing the frequency of CD4_{SOCS3} cells in the test population to the average frequency of CD4_{SOCS3} cells in the one or more reference populations; and (e) selecting the subject as the subject amendable to the treatment if the frequency of CD4_{SOCS3} cells in the test population is lower than the average frequency of CD4_{SOCS3} cells in the one or more reference populations.

Provided herein is a method of treating cancer in a subject having or suspected of having the cancer with one or more immune checkpoint inhibitors, the method including: (a) isolating a test population of PBMCs from the subject and one or more reference populations of PBMCs from one or more subjects having or suspected of having cancer, wherein each reference population can be isolated independently from each subject of the one or more subjects; (b) quantifying frequency of CD4_{SOCS3} cells in the test population; (c) quantifying frequency of CD4_{SOCS3} cells in the each reference population independently, and averaging the frequency of CD4_{SOCS3} cells in the one or more reference populations; (d) comparing the frequency of CD4_{SOCS3} cells in the test population to the average frequency of CD4_{SOCS3} in the one or more reference populations; and (e) administering a therapeutically effective amount of the one or more immune checkpoint inhibitors to the subject if the frequency of CD4_{SOCS3} cells in the test population is lower than the average frequency of CD4_{SOCS3} cells in the one or more reference populations, thereby treating the cancer in the subject.

In some embodiments, the frequency of CD4_{SOCS3} cells can be correlated with the frequency of CD14_{CTX} cells in any of the methods provided herein. In some embodiments, the lower frequency of CD4_{SOCS3} cells can lead to higher disease-free survival (DFS). In some embodiments, the frequency of CD4_{SOCS3} cells can be determined by sequencing PBMCs. In some embodiments, the sequencing method can be single cell RNA sequencing (scRNAseq), single cell cellular indexing of transcriptomes or epitopes by sequencing (CITE-seq). In some embodiments, the one or more immune checkpoint inhibitors can target PD-1/PD-L1 pathway. In some embodiments, the one or more immune checkpoint inhibitors targeting PD-1/PD-L1 pathway can be selected from the group consisting of AMP-224, and AMP-514 (MEDI-0680), atezolizumab (TECENTRIQ®), avelumab (BAVENCIO®), BI-754091, budigalimab (ABBV-181), camrelizumab (SHR-1210), cemiplimab (LIBTAYO®), cosibelimab (CK-301), dostarlimab (Jemperli), durvalumab (IMFINZI®), INCMGA00012 (MGA012), JTX-4014, nivolumab (OPDIVO®), pembrolizumab (KEYTRUDA®), pidilizumab (CT-011), retifanlimab (MGA012), sasanlimab (PF-06801591), sintilimab (IBI308), spartalizumab (PDR001), tislelizumab (BGB-A317), toripalimab (JS 001), and zimberelimab (AB122). In some embodiments, the one or more immune checkpoint inhibitors can be pembrolizumab (Keytruda®). In some embodiments, the treatment can include one or more therapeutic agents. In some embodiments, the one or more therapeutic agents can be GM-CSF. In some embodiments, the PBMCs can be isolated before the treatment is administered. In some embodiments, the PBMCs can be isolated after at least one cycle of the treatment is administered. In some embodiments, the PBMCs can be isolated at least one week, at least two weeks, at least 3 weeks, at least 4 weeks, at least 5 weeks, or at least 6 weeks after the treatment is administered. In some embodiments, the PBMCs can be isolated at least three weeks after the treatment is administered.

In any of the methods provided above, the method can further include (f) isolating a test group of intratumoral myeloid cells from the subject and one or more reference groups of intratumoral myeloid cells from the one or more subjects, wherein each reference group can be isolated independently from each subject of the one or more subjects; (g) quantifying frequency of Mac_{SPP1} cells in the test group; (h) quantifying frequency of Mac_{SPP1} cells in the each reference group independently, and averaging the frequency of Mac_{SPP1} cells in the one or more reference groups; and (i) comparing the frequency of Mac_{SPP1} cells from the test group to the average frequency of Mac_{SPP1} cells from the one or more reference groups. In some embodiments, the frequency of Mac_{SPP1} cells in the test group can be lower than the average frequency of Mac_{SPP1} cells in the one or more reference groups.

Provided herein is a method of selecting a subject amendable to treatment with one or more immune checkpoint inhibitors from one or more subjects having or suspected of having cancer, the method including: (a) isolating a test group of intratumoral myeloid cells from the subject and one or more reference groups of intratumoral myeloid cells from the one or more subjects, wherein each reference group can be isolated independently from each subject of the one or more subjects; (b) quantifying frequency of Mac_{SPP1} cells in the test group; (c) quantifying frequency of Mac_{SPP1} cells in the each reference group independently, and averaging the frequency of Mac_{SPP1} cells in the one or more reference groups; (d) comparing the frequency of Mac_{SPP1} cells from the test group to the average frequency of Mac_{SPP1} cells from the one or more reference groups; and (e) selecting the subject as the subject amendable to the treatment if the frequency of Mac_{SPP1} cells in the test group is lower than the average frequency of Mac_{SPP1} cells in the one or more reference groups.

Provided herein is a method of treating cancer in a subject having or suspected of having the cancer with one or more immune checkpoint inhibitors, the method including: (a) isolating a test group of intratumoral myeloid cells from the subject and one or more reference groups of intratumoral myeloid cells from the one or more subjects, wherein each reference group can be isolated independently from each subject of the one or more subjects; (b) quantifying frequency of Mac_{SPP1} cells in the test group; (c) quantifying frequency of Mac_{SPP1} cells in the each reference group independently, and averaging the frequency of Mac_{SPP1} cells in the one or more reference groups; (d) comparing the frequency of Mac_{SPP1} cells from the test group to the average frequency of Mac_{SPP1} cells from the one or more reference groups; and (e) administering a therapeutically effective amount of the one or more immune checkpoint inhibitors to the subject if the frequency of Mac_{SPP1} cells from the test group is lower than the average frequency of Mac_{SPP1} cells from the one or more reference groups, thereby treating the cancer in the subject.

In any of the methods described herein, the cancer can be selected from the group consisting of biliary tract cancer, prostate cancer, colon cancer, kidney cancer, and skin cancer.

Each of the aspects and embodiments described herein are capable of being used together, unless excluded either explicitly or clearly from the context of the embodiment or aspect.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1E collectively illustrates analysis of circulating immune cells in cancer-free subjects and biliary tract cancer (BTC) patients.

FIG. 1A is a schematic of experimental design for analyzing circulating immune cells in cancer-free subjects and BTC patients.

FIG. 1B is a Uniform Manifold Approximation and Projection (UMAP) plot of all cells from BTC patients and cancer-free subject blood samples colored by cell type. NK/NKT cluster contains T cells, NK T cells, and NK cells; cDC=conventional dendritic cells; mono=monocytes; pDC=plasmacytoid dendritic cells.

FIG. 1C is a graph illustrating percent of each cell type out of total immune cells in BTC patients (prior to treatment) and cancer-free subjects. * denotes significance (adjusted p<0.05). Boxes denote inter-quartile range (IQR) while bars denote 25%-1.5×IQR and 75%+1.5×IQR.

FIG. 1D is UMAP colored by density by population type (BTC=biliary cancer patients, Healthy=cancer-free subjects).

FIG. 1E is UMAP of all immune cells colored by protein and RNA expression for PD-1 (PDCD1), PD-L1 (CD274), and PD-L2 (PDCD1LG2).

FIGS. 2A-2E collectively illustrates dynamics of circulating immune cells with anti-PD-1 treatment in clinical responders and non-responders.

FIG. 2A is a schematic of experimental design for dynamics of circulating immune cells with anti-PD-1 treatment in clinical responders and non-responders.

FIG. 2B is a UMAP plot of all circulating immune cells from BTC patient and cancer-free subject samples colored by cell type. NK/NKT=T cells, NK T cells, and NK cells; cDC=conventional dendritic cells; mono=monocytes; pDC=plasmacytoid dendritic cells.

FIG. 2C is UMAP colored by density of cells by time-point for patients whose tumor responded to immunotherapy (responder) and whose tumor did not respond (non-responder).

FIG. 2D is a graph illustrating percent of each cell type out of total immune cells in responders and non-responders prior to anti-PD-1 treatment.

FIG. 2E is a graph illustrating percent of each cell type out of total immune cells in responders and non-responders three weeks following anti-PD-1 treatment. * denotes significance (adjusted p<0.05). Boxes denote inter-quartile range (IQR), while bars denote 25%-1.5×IQR and 75%+1.5×IQR.

FIGS. 3A-3F collectively illustrates circulating myeloid populations in BTC patients and cancer-free subjects.

FIG. 3A is a UMAP plot colored by myeloid cell subtype. cDC=conventional dendritic cells; mono=monocytes; pDC=plasmacytoid dendritic cells.

FIG. 3B is UMAP of myeloid cells showing expression of each protein or RNA molecule used to annotate myeloid subtypes.

FIG. 3C is a heatmap with expression of genes in the top enriched pathways (right labels) for each monocyte sub-type.

FIG. 3D is UMAP colored by density of cells within BTC patients and cancer-free subjects for all myeloid cells.

FIG. 3D is UMAP of CD68 and CD63 RNA expression across all myeloid cells.

FIG. 3F is a graph illustrating percent of each cell subtype out of total myeloid cells in BTC patients (prior to treatment) and cancer-free subjects. * denotes significance (adjusted p<0.05); *** denotes adjusted p-value <0.001. Boxes denote inter-quartile range (IQR) while bars denote 25%-1.5×IQR and 75%+1.5×IQR.

FIGS. 4A-4F collectively illustrates monocyte sub-types associated with anti-PD-1 treatment response.

FIG. 4A is graph showing trajectory analysis of monocyte sub-types from BTC patients and cancer-free subjects. Cells were ordered in pseudotime.

FIG. 4B is graph showing trajectory analysis of monocyte sub-types from BTC patients and cancer-free subjects. Cells were ordered in pseudotime with monocyte subtype overlaid.

FIG. 4C is graph showing trajectory analysis of monocyte sub-types from BTC patients and cancer-free subjects. Cells were ordered in pseudotime with response status overlaid.

FIG. 4D is a heatmap of differentially expressed genes along pseudotime, arranged by clusters of patterns of gene expression across pseudotime (direction shown by arrow).

FIG. 4E is a graph illustrating percent of each cell subtype out of total myeloid cells in BTC responders and non-responders prior to anti-PD-1 treatment.

FIG. 4F is a graph illustrating percent of each cell subtype out of total myeloid cells in BTC responders and non-responders 3 weeks following anti-PD-1 treatment. * denotes significance (adjusted p<0.05); ** denotes adjusted p-value <0.005; *** denotes adjusted p-value <0.001. Boxes denote inter-quartile range (IQR) while bars denote 25%-1.5×IQR and 75%+1.5×IQR.

FIGS. 5A-5D collectively illustrates monocyte gene signatures associated with poor prognosis in immune check point inhibitor insensitive cancer types.

FIG. 5A is a volcano plot of log 2 (fold change) and −log₁₀ (p-value) showing differently expressed genes between CD14_{CTX} and CD 14APC.

FIG. 5B is graphs illustrating expression of suppressive chemokines and cytokines associated with MDSC and M2 macrophages for CD14_{CTX} and CD14APC.

FIG. 5C is UMAP illustrating protein and RNA expression, overlaid of myeloid cells for HAVCR2 (Tim3) and ITGB1 (CD29, integrin-β1) and for the combination of both genes/proteins.

FIG. 5D is bar plots of each myeloid population gated on CD68 and calculated as percentage of total CD45⁺circulating immune cells as analyzed by flow cytometry of peripheral blood samples from cancer-free subjects (“Healthy”) or BTC patients. *=p<0.05, error bars denote standard deviation.

FIGS. 6A-6C collectively illustrates overall survival for different types of cancer.

FIG. 6A is a graph illustrating Kaplan-Meier curve of overall survival for cholangiocarcinoma cases in the TCGA dataset by high (solid line: median expression greater than composite score (CS)) or low (dashed line: median expression lower than composite score (CS)) expression of the CD14crx gene signature. NR=not reached, CI=confidence interval, OS=overall survival, DFS=disease-free survival.

FIG. 6B is a graph illustrating Kaplan-Meier curve of overall survival for colon cancer cases in the TCGA dataset by high (solid line: median expression greater than composite score (CS)) or low (dashed line: median expression lower than composite score (CS)) expression of the CD14_{CTX} gene signature. NR=not reached, CI=confidence interval, OS=overall survival, DFS=disease-free survival.

FIG. 6C is a graph illustrating Kaplan-Meier curve of overall survival for prostate cancer cases in the TCGA dataset by high (solid line: median expression greater than composite score (CS)) or low (dashed line: median expression lower than composite score (CS)) expression of the CD14_{CTX} gene signature. NR=not reached, CI=confidence interval, OS=overall survival, DFS=disease-free survival.

FIGS. 7A-7C collectively illustrates CD14_{CTX} are associated with CD4_{SOCS3} cells.

FIG. 7A is a UMAP plot of all T cells in cancer-free subjects and BTC patients colored by cell annotations.

FIG. 7B is a heatmap of Pearson correlation coefficients for cell type frequencies for myeloid and T cell sub-types.

FIG. 7C is graphs illustrating the frequency of the specified cell type out of total myeloid or T cells calculated and correlated as shown in each plot. Each dot corresponds to an individual patient sample.

FIGS. 8A-8E collectively illustrates CD14_{CTX} can induce CD4⁺ T cell suppression.

FIG. 8A is a schematic of co-culture conditions. The monocyte populations indicated were cultured with naïve healthy T cells for 6 days and re-stimulated with anti-CD3/CD28 beads for 3 days prior to harvest.

FIG. 8B is graphs illustrating CFSE staining shown for each co-culture condition. Cells were gated on CD45 CD3⁺CD4⁺. Stim=stimulated.

FIG. 8C is graphs illustrating flow cytometry assessment for SOCS3 expression in naive CD4⁺ T cells co-cultured with the indicated myeloid cell are shown (top panel). Results were representative of 3 experiments. Median fold change in SOCS3 expression for each condition compared to the T cell alone control from the combined 3 independent experiments (bottom panel).

FIG. 8D is graphs illustrating flow cytometry assessment from representative BTC peripheral blood mononuclear cells (PBMCs) sample, demonstrating SOCS3 and cytokine staining in stimulated (top panels) or unstimulated T cells (bottom). Results were representative of 3 experiments. BV=brilliant violet, AF=Alexa Fluor, PE=phycoerythrin.

FIG. 8E is immunohistochemistry staining for CD3, CD4, and SOCS3, shown individually and overlaid (overlay: T cells, white arrows=areas of co-staining) and overlaid with HAVCR2, SPP1, and CD68 (overlay: all, orange arrows=areas of co-localization of myeloid and T cells of interest) in biliary tumor tissue.

FIGS. 9A-9F collectively illustrating characterization of circulating immune cells with CITEseq.

FIG. 9A is a plot illustrating expression of proteins used to classify immune cell clusters, shown by percentage of cells with expression above the zero threshold (dot size) and mean expression (color intensity).

FIG. 9B is a plot illustrating characterization of circulating immune cells. For each immune cell class, expression of each gene is shown using a standard scale (for each gene, minimum is subtracted and then divided by its maximum).

FIG. 9C is a correlation plot of transcript (y-axis) and corresponding protein expression (x-axis) by pseudobulk expression data for all immune cells in the dataset. Legend shows value for the Spearman correlation coefficient.

FIG. 9D is a plot illustrating protein (left panels) and transcript (right panels) expression overlaid on UMAP plots for CD4.

FIG. 9E is a plot illustrating protein (left panels) and transcript (right panels) expression overlaid on UMAP plots for CD14.

FIG. 9F is a graph illustrating percent of each cell type out of total immune cells in responders and non-responders one week following anti-PD-1 administration. Boxes denote inter-quartile range (IQR), while bars denote 25%-1.5×IQR and 75%+1.5×IQR.

FIGS. 10A-10B collectively illustrates fluorescence-assisted cell sorting for isolation and co-culture of monocytes sub-populations from BTC patients and cancer-free subjects.

FIG. 10A is a sorting strategy for isolating myeloid cells from blood samples of cancer-free subjects.

FIG. 10B is a fluorescence-assisted cell sorting strategy for isolating myeloid cells from blood samples of BTC patents.

FIGS. 11A-11B collectively illustrates that monocyte sub-populations are differentially distributed in pseudotime and dynamic over time with the treatment with an immune checkpoint inhibitor.

FIG. 11A is plots illustrating monocyte sub-populations are differentially distributed in pseudotime and dynamic over time with treatment. Monocyte sub-populations are shown ordered in pseudotime for each time-point/response category.

FIG. 11B is a graph illustrating percent of each cell type out of all myeloid cells in responders and non-responders one week following anti-PD-1 administration. Boxes denote inter-quartile range (IQR) while bars denote 25%-1.5×IQR and 75%+1.5×IQR.

FIGS. 12A-12C collectively illustrates responders and non-responders to the treatment with an immune checkpoint inhibitor have diverging myeloid sub-populations.

FIG. 12A is a plot illustrating checkpoint inhibitor (CPI) responders and non-responders have diverging myeloid sub-populations. Immune pathways are plotted as a network with genes assigned to each pathway and differentially expressed in CD14_{APC}. Number of genes in each pathway is shown by the size of the pathway circle and fold change is shown by the heatmap in the legend.

FIG. 12B is a plot illustrating CPI responders and non-responders have diverging myeloid sub-populations. Immune pathways are plotted as a network with genes assigned to each pathway and differentially expressed in CD14_{CTX}.

FIG. 12C is a bar plots of each myeloid population gated on CD14 and calculated as percentage of total CD45⁺ circulating immune cells as analyzed by flow cytometry of peripheral blood cells from BTC patients and cancer-free subject. *=p<0.05, error bars denote standard deviation.

FIGS. 13A-13E collectively illustrates that intra-tumoral BTC myeloid populations correlate with circulating CD14_{CTX} cells.

FIG. 13A is UMAP colored by myeloid cell sub-types present in biliary cancer tumor dataset, illustrating intra-tumoral BTC myeloid populations correlate with circulating CD14_{CTX}.

FIG. 13B is a plot illustrating expression of genes for phenotypic markers, shown for myeloid cell sub-types in the tumor dataset using a standard scale (for each gene, minimum is subtracted and then divided by its maximum).

FIG. 13C is a heatmap of Pearson correlation coefficient values for gene signatures using pseudobulk gene expression data for each myeloid cell sub-type from peripheral blood and intra-tumoral datasets. Legend shows R value for each correlation.

FIG. 13D is a heatmap showing mean expression of CD 14_{CTX} hallmark genes in myeloid cells isolated from biliary tumors.

FIG. 13E is immunohistochemistry staining for CD68, SPP1 and HAVCR2 in biliary tumor tissue (two different representative patient samples are shown) and negative control (tonsil tissue). Examples of CD68 HAVCR2⁺SPP1⁺ cells (arrowheads) are demarcated in overlay image.

FIGS. 14A-14D collectively illustrates peripheral T cell characterization and association with frequency of myeloid cell sub-types

FIG. 14A is a plot illustrating mean expression of each gene is shown using a standard scale for each T cell type.

FIG. 14B is a plot illustrating expression of each protein, shown by percentage of cells with expression above the zero threshold (dot size) and mean expression (color intensity) for each T cell type.

FIG. 14C is a plot illustrating protein expression of CD45RA (x-axis) and CCR7 (y-axis) for individual cells in the specified T cell types, demonstrating examples of using protein data to annotate T cells as naïve, effector, and memory phenotypes.

FIG. 14D is a plot illustrating the frequency of the specified cell type out of total myeloid or T cells, calculated and correlated as shown in each plot. Each dot corresponds to an individual patient sample.

FIGS. 15A-15C collectively illustrates that CD3⁺CD4⁺SOCS3⁺ cells are identified in biliary tumors and co-localize with CD68 HAVCR2⁺SPP1⁺ cells.

FIG. 15A is UMAP colored by cell annotations for intra-tumoral T cells.

FIG. 15B is a plot illustrating mean expression of each gene for each intra-tumoral T cell type, shown using a standard scale.

FIG. 15C is immunohistochemistry staining for SOCS3, CD4, and CD3, shown individually and overlaid (overlay: T cells), and with overlay of staining for CD68, HAVCR2, and SPP1 (overlay: all) in a representative biliary tumor (different patient from staining displaying in main text). Examples of CD3⁺CD4⁺SOCS3⁺ cells and CD68⁺ cells (white arrowheads) and of co-localization of CD3⁺CD4⁺SOCS3⁺ cells with CD68⁺HAVCR2⁺SPP1⁺ cells (orange arrowheads) are highlighted in overlay image. Negative control staining for SOCS3 probe with immunofluorescence for CD68 and CD4 in a control tonsil tissue.

### DETAILED DESCRIPTION

The present disclosure relates to, inter alia, characterizing circulating monocytes, which can induce T cell paralysis and can lead to resistance to the treatment with one or more immune checkpoint inhibitors in cancer patients. Provided herein are methods of determining and/or selecting whether a subject having or suspected of having cancer will be amendable to treatment with one or more immune checkpoint inhibitors. Also provided herein are methods of treating cancer in a subject having or suspected of having the cancer with a therapeutically effective amount of one or more immune checkpoint inhibitors by specifically selecting the subject who will respond to the checkpoint inhibitor (CPI) treatment. The embodiments of the present disclosure are described in greater details below.

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular embodiments described herein and as such can vary. Those of skill in the art will recognize that there are variations and modifications of the present disclosure, which are encompassed within its scope.

### Definition

All terms are intended to be understood as they would be understood by a person skilled in the art. Unless defined otherwise, all technical and scientific terms used herein have the same meaning as commonly understood by one of ordinary skill in the art to which the disclosure pertains.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein

The following definitions supplement those in the art and are directed to the current application and are not to be imputed to any related or unrelated cases, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a”, “an”, and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C”. The term “or” can be used conjunctively or disjunctively, unless the context specifically refers to a disjunctive use.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, an “individual” or a “subject” includes animals, such as human (e.g., human individuals) and non-human animals. In some embodiments, an “individual” or “subject” can be a patient under the care of a physician. Thus, the subject can be a human patient or an individual who has, can be at risk of having, or can be suspected of having a disease of interest (e.g., cancer) and/or one or more symptoms of the disease. The subject can also be an individual who is diagnosed with a risk of the condition of interest at the time of diagnosis or later. The term “non-human animals” can include all vertebrates, e.g., mammals, e.g., rodents, e.g., mice, non-human primates, and other mammals, such as e.g., sheep, dogs, cows, chickens, and non-mammals, such as amphibians, reptiles, etc.

The term “treating” or “treatment” of a condition as used herein can include preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. With regard to cancer, “treating” or “treatment” can refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” can include eradicating all or part of a tumor, inhibiting or slowing tumor growth and metastasis, preventing or delaying the development of a tumor, or some combination thereof.

The term “therapeutically effective amount” or any grammatical equivalent thereof as used herein refers to the dosage or concentration of a drug (e.g., a CPI) effective to treat a disease or a condition, such as cancer. For example, with regard to the use of an immune checkpoint inhibitor to treat e.g., cancer, a therapeutically effective amount is the dosage or concentration of the immune checkpoint inhibitor capable of eradicating all or part of a tumor or cancer, inhibiting or slowing tumor or cancer growth, inhibiting growth or proliferation of cells mediating a cancerous condition, inhibiting tumor cell metastasis, ameliorating any symptom or marker associated with a tumor or cancerous condition, preventing or delaying the development of a tumor or cancerous condition, or some combination thereof. An appropriate amount in any given instance can be ascertained by those skilled in the art or capable of determination by routine experimentation.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### Overview

The myeloid component of the immune system contains both tumor-promoting and tumor-suppressing subsets that function in inflammation and cancer immunity. While the effects of checkpoint inhibitor (CPI) on T cells are well documented, the effects of CPI treatment on myeloid cells are not well understood despite associations to their altered frequency and activation states. For example, an increased frequency of circulating CD14⁺CD16 HLADR^{hi} monocytes prior to treatment, along with a decreased frequency of T cells, correlate with survival and response to an anti-PD-1 treatment in melanoma patients. Further, PD-1 signaling can polarize macrophages to a M2 phenotype and leads to impaired phagocytosis. Although suppressive myeloid cells have been proposed as a mechanism of resistance to cancer immunotherapy, their role in response to an immune checkpoint inhibitor treatment in refractory cancers, such as biliary tract cancer (BTC), is largely unknown. A refractory cancer refers to a cancer that is not amendable to treatment(s), either initially unresponsive to treatment(s) or become unresponsive over time.

The present disclosure provides important insights into the circulating immune system of cancer patients and mechanisms of responses and insensitivity to an immune checkpoint inhibitor treatment (e.g., an anti-PD-1 treatment, such as, but not limited to, pembrolizumab). The present disclosure identifies an immunosuppressive myeloid sub-population (i.e., CD14_{CTX} cells) and its gene signature (i.e., CD14crx gene signature), which can be correlated with poor prognosis in cancer patients. Targeting this immunosuppressive myeloid sub-population in combination with one or more CPIs presents a new avenue for overcoming CPI insensitivity and improving outcomes in patients with cancer.

### Methods of the Disclosure

The present disclosure relates to, inter alia, characterizing circulating monocytes, which can induce T cell paralysis and can lead to resistance to the treatment with one or more checkpoint inhibitors (CPIs) in cancer patients. Currently, there exists no effective predictive factors elucidating whether a cancer patient will respond to and benefit from a CPI treatment. The present disclosure provides new ways of determining predictive value of CPI treatments, which can enable selecting cancer patients who will respond to and benefit from a CPI treatment, prior to treating the cancer patients with one or more CPIs.

Accordingly, provided herein are methods of determining and/or selecting whether a subject having or suspected of having cancer will be amendable to treatment with one or more immune checkpoint inhibitors (e.g., a PD-1 inhibitor, such as, but not limited, to pembrolizumab). Also provided herein are methods of identifying a subject having or suspected of having cancer who will be amendable to treatment with one or more CPIs (e.g., a PD-1 inhibitor, such as, but not limited, to pembrolizumab). Also provided herein are methods of treating cancer in a subject having or suspected of having the cancer with a therapeutically effective amount of one or more CPIs (e.g., an anti-PD-1 treatment, such as, but not limited, to pembrolizumab) by specifically selecting the subject who will respond to the one or more immune checkpoint inhibitors.

As non-limiting embodiments, the Examples described herein utilized multiplexed single-cell transcriptomic and epitope sequencing method to profile over 200,000 peripheral blood mononuclear cells (PBMCs) from advanced biliary track cancer (BTC) patients and matched cancer-free subjects. The results of the studies in the present disclosure demonstrate that CD14⁺ monocytes expressing high levels of immunosuppressive cytokines and trafficking molecules involved in chemotaxis (CD14_{CTX}) can be associated with resistance to treatment with one or more CPIs, such as, but not limited to, an anti-PD-1 treatment (e.g., pembrolizumab). Furthermore, CD14_{CTX} can directly suppress CD4⁺ T cells and induce SOCS3 expression in naïve CD4⁺ T cells rendering them functionally unresponsive. As illustrated in the present disclosure, gene signatures from CD14_{CTX} can be correlated with worse survival in BTC patients as well as in other immune checkpoint inhibitor refractory cancers, such as, but not limited to, biliary tract cancer, prostate cancer, colon cancer, gastric cancer, gastroesophageal junction adenocarcinoma, esophageal cancer, kidney cancer, skin cancer, lung cancer, pancreatic cancer, liver cancer, head-and-neck cancer, mesothelioma, cervical cancer, ovarian cancer, endometrial cancer, uterine cancer, breast cancer, testicular cancer, gall bladder cancer, heart cancer, glandular cancer, brain cancer, or thyroid cancer. In some embodiments, the cancer can be a solid tumor. In some embodiments, the cancer can be a hematological cancer. Exemplary hematological cancer can include, but are not limited to, leukemias, lymphomas, or myelomas. The results presented herein demonstrate that monocytes arising in the setting of immune checkpoint inhibitor insensitivity can induce T cell paralysis as a distinct mode of tumor-mediated immunosuppression.

### Methods of Selecting a Subject Amendable to CPI Treatments

Provided herein, inter alia, methods of selecting a subject amendable to treatment(s) with one or more immune checkpoint inhibitors (i.e., CPI treatment) from one or more subjects having or suspected of having cancer.

For example, the method can include (a) isolating a test population of PBMCs from the subject and one or more reference populations of PBMCs from each subject of the one or more subjects; (b) quantifying frequency of CD14_{CTX} cells in the test population; (c) quantifying frequency of CD14_{CTX} cells in each reference population independently, and averaging the frequency CD14crx cells in the one or more reference populations; (d) comparing the frequency of CD14_{CTX} cells in the test population to the average frequency of CD14_{CTX} in the one or more reference population; and (d) selecting the subject as the subject amendable to the treatment if the frequency of CD14_{CTX} cells in the test population is lower than the average frequency of CD14_{CTX} cells in the one or more reference populations.

In another example, the method can include (a) isolating a test population of PBMCs from the subject and one or more reference populations of PBMCs from each subject of the one or more subjects; (b) quantifying frequency of CD4_{SOCS3} cells in the test population; (c) quantifying frequency of CD4_{SOCS3} cells in each reference population independently, and averaging the frequency CD4_{SOCS3} cells in the one or more reference populations; (d) comparing the frequency of CD4_{SOCS3} cells in the test population to the average frequency of CD4_{SOCS3} cells in the one or more reference populations; and (e) selecting the subject as the subject amendable to the treatment if the frequency of CD4_{SOCS3} cells in the test population is lower than the average frequency of CD4_{SOCS3} cells in the one or more reference populations.

In any of the methods provided herein, PBMCs can be isolated using any technique known to the skilled artisan. For example, PBMCs can be isolated per an established institutional protocol, as described in Example 1.

In some embodiments, any of the methods provided herein can further include (f) isolating a test group of intratumoral myeloid cells from the subject and one or more reference groups of intratumoral myeloid cells from the one or more subjects; (g) quantifying frequency of Mac_{SPP1} cells in the test group; (h) quantifying frequency of Mac_{SPP1} cells in each reference group independently, and averaging the frequency of Mac_{SPP1} cells in the one or more reference groups; (i) comparing the frequency of Mac_{SPP1} cells from the test group to the average frequency of Mac_{SPP1} cells from the one or more reference groups. In some embodiments, the frequency of Mac_{SPP1} cells can be correlated with the frequency of CD14_{CTX} cells. In some embodiments, the frequency of Mac_{SPP1} cells can be correlated with the frequency of CD4_{SOCS3} cells. In some embodiments, the frequency of Mac_{SPP1} cells in the test group can be lower than the average frequency of Mac_{SPP1} cells in the one or more reference groups.

In some embodiments, the lower frequency of CD14_{CTX} cells can lead to higher disease-free survival (DFS). In some embodiments, the lower frequency of CD4_{SOCS3} cells can lead to higher disease-free survival (DFS). In some embodiments, the frequency of CD14_{CTX} cells and/or CD4_{SOCS3} cells can be determined by sequencing PBMCs. In some embodiments, the sequencing method can be single cell RNA sequencing (scRNAseq), single cell cellular indexing of transcriptomes or epitopes by sequencing (CITE-seq).

In some embodiments, the one or more immune checkpoint inhibitors can target PD-1/PD-L1 pathway. In some embodiments, the one or more immune checkpoint inhibitors targeting PD-1/PD-L1 pathway is selected from the group consisting of AMP-224, and AMP-514 (MEDI-0680), atezolizumab (e.g., TECENTRIQ®), avelumab (e.g., BAVENCIO®), BI-754091, budigalimab (ABBV-181), camrelizumab (SHR-1210), cemiplimab (e.g., LIBTAYO®), cosibelimab (CK-301), dostarlimab (Jemperli), durvalumab (e.g., IMFINZI®), INCMGA00012 (MGA012), JTX-4014, nivolumab (e.g., OPDIVO®), pembrolizumab (e.g., KEYTRUDA®), pidilizumab (CT-011), retifanlimab (MGA012), sasanlimab (PF-06801591), sintilimab (IBI308), spartalizumab (PDR001), tislelizumab (BGB-A317), toripalimab (JS 001), and zimberelimab (AB122). In some embodiments, the one or more immune checkpoint inhibitors can be pembrolizumab (Keytruda®).

In some embodiments, a CPI treatment can further include one or more therapeutic agents, wherein the one or more therapeutic agents is not an immune checkpoint inhibitor. In some embodiments, the one or more therapeutic agents can be GM-CSF. In some embodiments, the one or more additional therapeutic agents can be a chemotherapeutic agent. Non-limiting examples of a chemotherapeutic agent are described elsewhere in the present disclosure.

In some embodiments, the PBMCs can be isolated before a CPI treatment is administered. In some embodiments, the PBMCs can be isolated at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks after the treatment is administered. In some embodiments, the PBMCs can be isolated at least 1 weeks after the treatment is administered. In some embodiments, the PBMCs can be isolated at least 3 weeks after the treatment is administered. In some embodiments, the PBMCs can be isolated after at least 1 cycle, at least 2 cycle, at least 3 cycle, at least 4 cycle, at least 5 cycle, at least 6 cycle, at least 7 cycle, at least 8 cycle, at least 9 cycle, at least 10 cycle, at least 11 cycle, or at least 12 cycle of the treatment is administered. In some embodiments, the PBMCs can be isolated after at least 1 cycle of the treatment is administered. In some embodiments, the PBMCs can be isolated after at least 2 cycle of the treatment is administered. In some embodiments, the PBMCs can be isolated after at least 3 cycle of the treatment is administered.

In some embodiments, a cell surface marker of the CD14crx cells can be selected from the group consisting of T cell immunoglobulin and mucin domain-containing protein 3 (Tim3), CD29 (integrin β1), CD14, CD63, and CD68. In some embodiments, the cell surface marker of the CD14_{CTX} cells can be Tim3. In some embodiments, the cell surface marker of the CD14_{CTX} cells can be CD29.

Also provided herein is a method of selecting a subject amendable to treatment with one or more immune checkpoint inhibitors, wherein the method can include (a) isolating a test group of intratumoral myeloid cells from the subject and one or more reference groups of intratumoral myeloid cells from the one or more subjects; (b) quantifying frequency of Mac_{SPP1} cells in the test group; (c) quantifying frequency of Mac_{SPP1} cells in each reference group independently, and averaging the frequency of Mac_{SPP1} cells in the one or more reference groups; (c) comparing the frequency of Mac_{SPP1} cells from the test group to the average frequency of Mac_{SPP1} cells from the one or more reference groups; and (d) selecting the subject as the subject amendable to the treatment if the frequency of Mac_{SPP1} cells in the test group is lower than the average frequency of Mac_{SPP1} cells in the one or more reference groups.

### Methods of Treating Cancer in a Subject Amendable to CPI Treatments

Provided herein, inter alia, methods of treating cancer in a subject having or suspected of having the cancer with one or more immune checkpoint inhibitors by specifically selecting the subject who will respond to a CPI treatment.

For example, the method can include (a) isolating a test population of PBMCs from the subject and one or more reference populations of PBMCs from one or more subjects having or suspected of having cancer; (b) quantifying frequency of CD14_{CTX} cells in the test population; (c) quantifying frequency of CD14_{CTX} cells in each reference population independently, and averaging the frequency of CD14_{CTX} cells in the one or more reference populations; (d) comparing the frequency of CD14_{CTX} cells in the test population to the average frequency of CD14_{CTX} in the one or more reference populations; and (e) administering a therapeutically effective amount of the one or more CPIs to the subject if the frequency of CD14_{CTX} cells in the test population is lower than the average frequency of CD14_{CTX} cells in the one or more reference populations, thereby treating the cancer in the subject.

In another example, the method can include (a) isolating a test population of PBMCs from the subject and one or more reference populations of PBMCs from one or more subjects having or suspected of having cancer; (b) quantifying frequency of CD4_{SOCS3} cells in the test population; (c) quantifying frequency of CD4_{SOCS3} cells in each reference population independently, and averaging the frequency of CD4_{SOCS3} cells in the one or more reference populations; (d) comparing the frequency of CD4_{SOCS3} cells in the test population to the average frequency of CD4_{SOCS3} in the one or more reference populations; and (e) administering a therapeutically effective amount of the one or more CPIs to the subject if the frequency of CD4_{SOCS3} cells in the test population is lower than the average frequency of CD4_{SOCS3} cells in the one or more reference populations, thereby treating the cancer in the subject.

In some embodiments, any of the methods provided herein can further include (f) isolating a test group of intratumoral myeloid cells from the subject and one or more reference groups of intratumoral myeloid cells from the one or more subjects; (g) quantifying frequency of Mac_{SPP1} cells in the test group; (h) quantifying frequency of Mac_{SPP1} cells in each reference group independently, and averaging the frequency of Mac_{SPP1} cells in the one or more reference groups; (i) comparing the frequency of Mac_{SPP1} cells from the test group to the average frequency of Mac_{SPP1} cells from the one or more reference groups. In some embodiments, the frequency of Mac_{SPP1} cells can be correlated with the frequency of CD14_{CTX} cells. In some embodiments, the frequency of Mac_{SPP1} cells can be correlated with the frequency of CD4_{SOCS3} cells. In some embodiments, the frequency of Mac_{SPP1} cells in the test group can be lower than the average frequency of Mac_{SPP1} cells in the one or more reference groups.

One or more CPIs can be administered in an effective regime meaning a dosage, route of administration and frequency of administration that delays the onset, reduces the severity, inhibits further deterioration, and/or ameliorates at least one sign or symptom of a disorder. If a subject is already suffering from a disorder, the regime can be referred to as a therapeutically effective regime. If the subject is at elevated risk of the disorder relative to the general population but is not yet experiencing symptoms, the regime can be referred to as a prophylactically effective regime. In some embodiments, therapeutic or prophylactic efficacy can be observed in a subject relative to historical controls or past experience in the same subject. In other embodiments, therapeutic or prophylactic efficacy can be demonstrated in a preclinical or clinical trial in a population of treated subjects relative to a control population of untreated subjects.

Administration can be parenteral, intravenous, oral, subcutaneous, intra-arterial, intracranial, intrathecal, intraperitoneal, intratumoral, topical, intranasal or intramuscular. In some embodiments, administration into the systemic circulation can be by intravenous or subcutaneous administration. Intravenous administration can be, for example, by infusion over a period such as 30-90 min. An appropriate time in any given circumstances can be ascertained by those skilled in the art.

The frequency of administration of the one or more immune checkpoint inhibitors (i.e., CPIs) depends on the half-life of the CPI in the circulation, the condition of the subject and the route of administration among other factors. The frequency can be daily, weekly, monthly, quarterly, or at irregular intervals in response to changes in the subject's condition or progression of the disorder being treated. In an embodiment, the frequency can be in two-week cycles. In another embodiment, the frequency can be in three-week cycles. In another embodiment, the frequency can be four-week cycles. In another embodiment, the frequency can be six-week cycles. An exemplary frequency for intravenous administration can be between weekly and quarterly over a continuous cause of treatment, although more or less frequent dosing can also be possible. For subcutaneous administration, an exemplary dosing frequency can be daily to monthly, although more or less frequent dosing is also possible.

In some embodiments, the subject selected for a CPI treatment can have a lower frequency of CD14_{CTX} cells and/or a lower frequency of CD4_{SOCS3} cells compared to one or more reference subjects. In a preferred embodiment, the subject selected for the CPI treatment is a responder (a subject whose tumors respond to treatment with an immune checkpoint inhibitor), and the one or more reference subject is a non-responder (a subject whose tumors respond to treatment with an immune checkpoint inhibitor). In some embodiments, a reference subject is one or more cancer-free subjects. In some embodiments, a reference subject is one or more subjects having or suspected of having the same type of cancer as the selected subject.

In some embodiments, the lower frequency of CD14_{CTX} cells can lead to higher disease-free survival (DFS). In some embodiments, the lower frequency of CD4_{SOCS3} cells can lead to higher disease-free survival (DFS). In some embodiments, the frequency of CD14_{CTX} cells and/or CD4_{SOCS3} cells can be determined by sequencing PBMCs. In some embodiments, the sequencing method can be single cell RNA sequencing (scRNAseq), single cell cellular indexing of transcriptomes or epitopes by sequencing (CITE-seq).

In some embodiments, the one or more CPIs can target PD-1/PD-L1 pathway. In some embodiments, the one or more CPIs targeting PD-1/PD-L1 pathway is selected from the group consisting of AMP-224, and AMP-514 (MEDI-0680), atezolizumab (e.g., TECENTRIQ®), avelumab (e.g., BAVENCIO®), BI-754091, budigalimab (ABBV-181), camrelizumab (SHR-1210), cemiplimab (e.g., LIBTAYO®), cosibelimab (CK-301), dostarlimab (Jemperli), durvalumab (e.g., IMFINZI®), INCMGA00012 (MGA012), JTX-4014, nivolumab (e.g., OPDIVO®), pembrolizumab (e.g., KEYTRUDA®), pidilizumab (CT-011), retifanlimab (MGA012), sasanlimab (PF-06801591), sintilimab (IBI308), spartalizumab (PDR001), tislelizumab (BGB-A317), toripalimab (JS 001), and zimberelimab (AB122). In some embodiments, the one or more CPIs can be pembrolizumab (Keytruda®).

In some embodiments, a CPI treatment can further include one or more therapeutic agents, wherein the one or more therapeutic agents is not an immune checkpoint inhibitor. In some embodiments, the one or more therapeutic agents can be GM-CSF. In some embodiments, the one or more additional therapeutic agents can be a chemotherapeutic agent. Non-limiting examples of a chemotherapeutic agent are described elsewhere in the present disclosure.

In some embodiments, the PBMCs can be isolated before a CPI treatment is administered. In some embodiments, the PBMCs can be isolated at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks after the treatment is administered. In some embodiments, the PBMCs can be isolated at least 1 weeks after the treatment is administered. In some embodiments, the PBMCs can be isolated at least 3 weeks after the treatment is administered. In some embodiments, the PBMCs can be isolated after at least 1 cycle, at least 2 cycle, at least 3 cycle, at least 4 cycle, at least 5 cycle, at least 6 cycle, at least 7 cycle, at least 8 cycle, at least 9 cycle, at least 10 cycle, at least 11 cycle, or at least 12 cycle of the treatment is administered. In some embodiments, the PBMCs can be isolated after at least 1 cycle of the treatment is administered. In some embodiments, the PBMCs can be isolated after at least 2 cycle of the treatment is administered. In some embodiments, the PBMCs can be isolated after at least 3 cycle of the treatment is administered.

In some embodiments, a cell surface marker of the CD14_{CTX} cells can be selected from the group consisting of T cell immunoglobulin and mucin domain-containing protein 3 (Tim3), CD29 (integrin β1), CD14, CD63, and CD68. In some embodiments, the cell surface marker of the CD14_{CTX} cells can be Tim3. In some embodiments, the cell surface marker of the CD14_{CTX} cells can be CD29.

Also provided herein is a method of treating cancer in a subject by selecting the subject who will respond to treatment with one or more CPIs, wherein the method includes (a) isolating a test group of intratumoral myeloid cells from the subject and one or more reference groups of intratumoral myeloid cells from the one or more subjects; (b) quantifying frequency of Mac_{SPP1} cells in the test group; (c) quantifying frequency of Mac_{SPP1} cells in each reference group independently, and averaging the frequency of Mac_{SPP1} cells in the one or more reference groups; (d) comparing the frequency of Mac_{SPP1} cells from the test group to the average frequency of Mac_{SPP1} cells from the one or more reference groups; and (e) administering a therapeutically effective amount of the one or more CPIs to the subject if the frequency of Mac_{SPP1} cells from the test group is lower than the average frequency of Mac_{SPP1} cells from the one or more reference groups, thereby treating the cancer in the subject.

### Immune Checkpoint Inhibitors (CPIs)

Non-limiting examples of immune checkpoints (ligands and receptors), some of which are selectively upregulated in various types of tumor cells, that can be candidates for blockade include PD-1 (programmed cell death protein 1); PD-L1 (programmed cell death ligand 1); PD-L2 (programmed cell death ligand 2); BTLA (B and T lymphocyte attenuator); CTLA4 (cytotoxic T-lymphocyte associated antigen 4); TIM-3 (T cell immunoglobulin mucin protein 3); LAG-3 (lymphocyte activation gene 3); TIGIT (T cell immunoreceptor with Ig and ITIM domains); and Killer Inhibitory Receptors, which can be divided into two classes based on their structural features: (i) killer cell immunoglobulin-like receptors (KIRs), and (ii) C-type lectin receptors (members of the type II transmembrane receptor family). Other less well-defined immune checkpoints have been well described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). Pardoll, Nature Rev. Cancer 12 (4): 252-264 (2012).

The present disclosure contemplates use of one or more inhibitors of the aforementioned immune checkpoint ligands and receptors, as well as any other immune checkpoint ligands and receptors. Certain modulators of immune checkpoints are currently approved, and many others are in development.

For example, approved anti-PD-1 antibodies can include nivolumab (e.g., OPDIVO®; Bristol Myers Squibb) and pembrolizumab (e.g., KEYTRUDA®; Merck) for various cancers, including squamous cell carcinoma, classical Hodgkin lymphoma and urothelial carcinoma. Approved anti-PD-L1 antibodies include avelumab (e.g., BAVENCIO®; EMD Serono & Pfizer), atezolizumab (e.g., TECENTRIQ®; Roche/Genentech), and durvalumab (e.g., IMFINZI®; AstraZeneca) for certain cancers, including urothelial carcinoma.

For example, approved anti CTLA-4 antibodies include ipilimumab (e.g., YERVOY®; Bristol Myers Squibb), a fully humanized CTLA-4 monoclonal antibody, and abatcept (e.g., ORENCIA®; Bristol Myers Squibb), a fusion protein composed of the Fc region of the immunoglobulin G1 (IgG1) fused to the extracellular domain of CTLA-4.

In some embodiment, an immune checkpoint inhibitor can include a PD-1 inhibitor, a PD-L1 inhibitor, a PD-L2 inhibitor, a CTLA-4 inhibitor, a TIM-3 inhibitor, a LAG-3 inhibitor, a TIGIT inhibitor, or any combination thereof. In some embodiments, an immune checkpoint inhibitor can be a fusion protein comprising a portion of an immunoglobulin protein and a portion of an immune checkpoint receptor or ligand.

In some embodiments, an immune checkpoint inhibitor can be selected from AMP-224, and AMP-514 (MEDI-0680), atezolizumab (e.g., TECENTRIQ®), avelumab (e.g., BAVENCIO®), BI-754091, budigalimab (ABBV-181), camrelizumab (SHR-1210), cemiplimab (e.g., LIBTAYO®), cosibelimab (CK-301), dostarlimab (Jemperli), durvalumab (e.g., IMFINZI®), INCMGA00012 (MGA012), JTX-4014, nivolumab (e.g., OPDIVO®), pembrolizumab (e.g., KEYTRUDA®), pidilizumab (CT-011), retifanlimab (MGA012), sasanlimab (PF-06801591), sintilimab (IBI308), spartalizumab (PDR001), tislelizumab (BGB-A317), toripalimab (JS 001), and zimberelimab (AB122).

The present disclosure encompasses pharmaceutically acceptable salts, acids, or derivatives any of the above.

### Circulating Myeloid Populations in Cancer Patients

Circulating and tissue resident myeloid cells are known to be heterogeneous in cancer patients, having immune-modulating functions ranging from being tumor promoting to tumor suppressing. An understanding of immunosuppressive capacity of myeloid-derived suppressor cells (MDSC), M2 macrophages, and tumor-associated macrophages (TAMs) is emerging, along with the heterogeneity of myeloid phenotypes within different tumor types.

The present disclosure describes circulating monocytes as a hallmark of cancer and of insensitivity to treatment with one or more CPIs, such as a PD-1 inhibitor (e.g., pembrolizumab). While these monocytes share some features of MDSC and M2 macrophages, they do not conform to these classifiers. The present disclosure provides and identifies new classifications of circulating myeloid sub-populations in cancer patients (i.e., CD14_{CTX}, CD14_{APC}, CD14_{IFL}, and CD14 _{ISG}) (see, for example, Example 4).

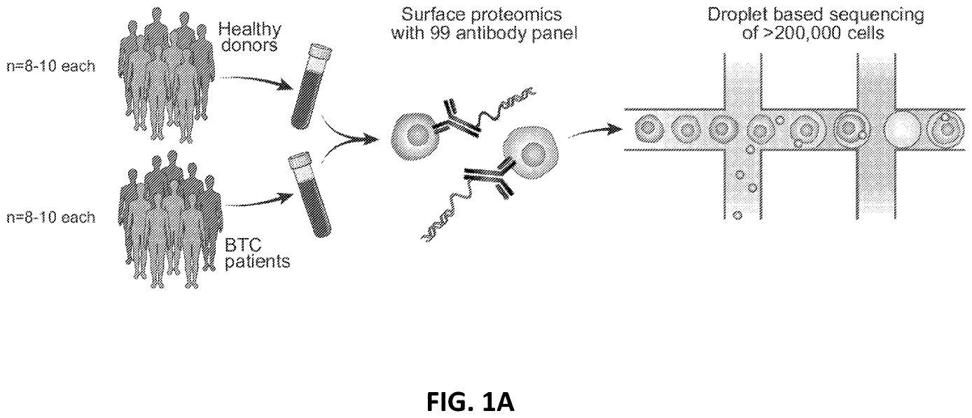

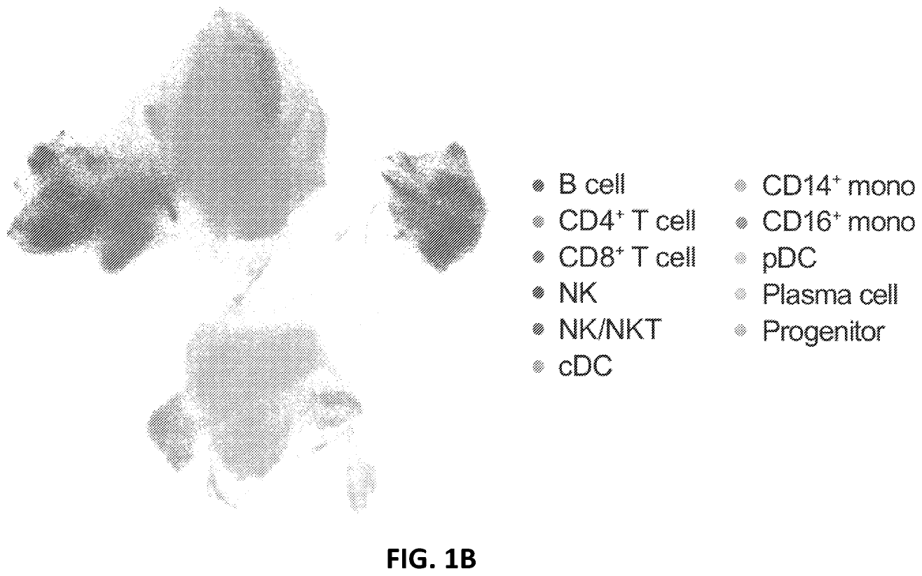

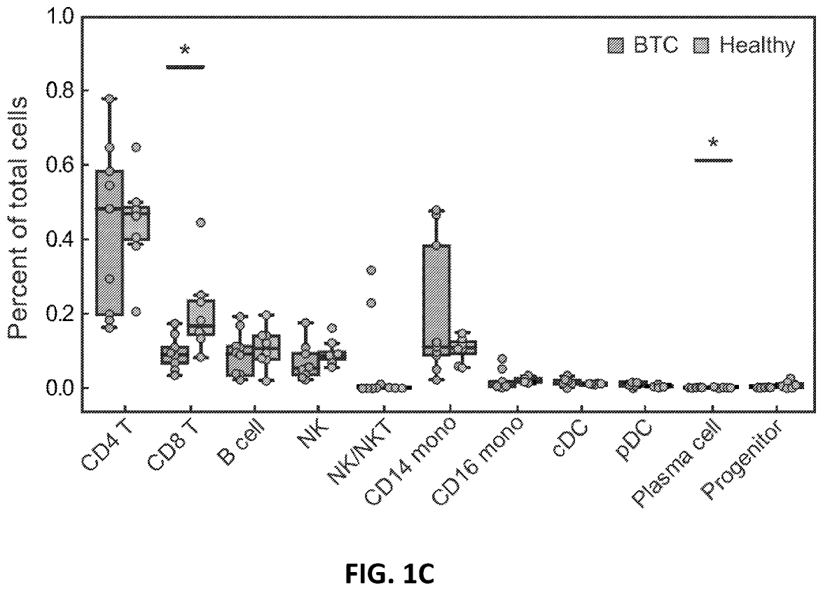

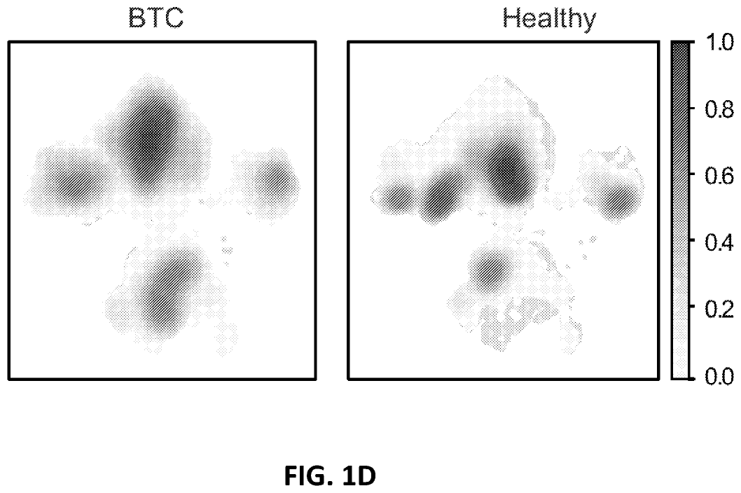

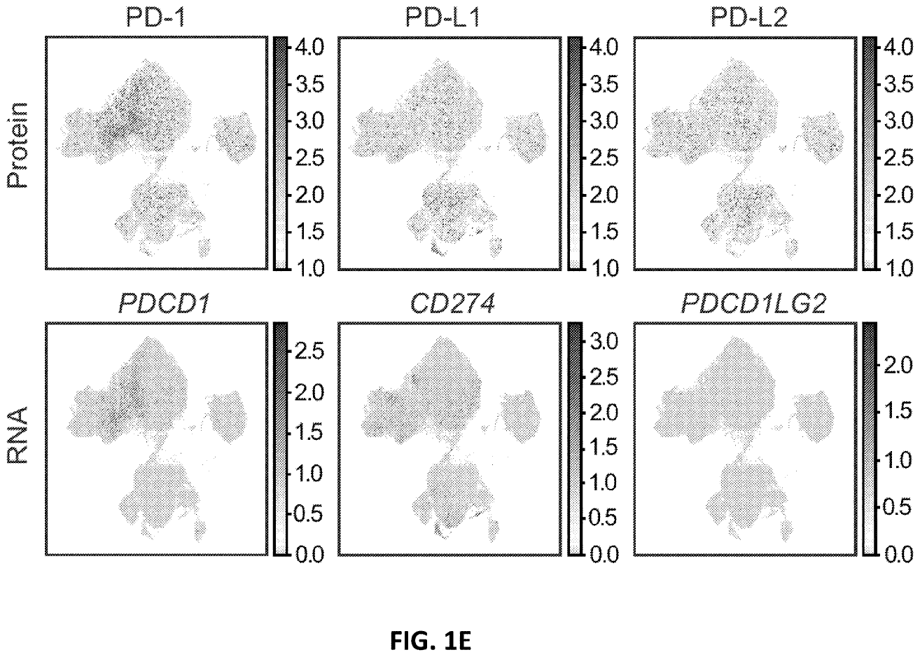

As described herein, peripheral blood mononuclear cells (PBMCs) can be isolated from a subject having or suspected of having cancer prior to or after receiving treatment of one or more CPIs. The canonical circulating myeloid and lymphoid cell types can include B cells, CD4⁺ and CD8⁺ T cells, NK cells, NK T cells, plasmacytoid and conventional dendritic cells (pDC and cDC), CD14⁺ and CD16⁺ monocytes, plasma cells, and a small immune progenitor cell population. Cancer-free subjects and cancer patients can have differences in the composition of broadly defined circulating immune cells (FIG. 1C). For example, baseline frequencies of CD8⁺ and plasma cells in cancer patients can be decreased compared to those of cancer-free subjects. In some embodiments, the transcriptional state of the monocyte compartment can be distinct between cancer patients and cancer-free subjects both pre-treatment and post-treatment, while no difference in frequency can be seen in the monocyte compartment overall (FIGS. 1C, 2D-2E, and 9F). In some embodiments, an immune checkpoint inhibitor, such as, but not limited to, an anti-PD-1 inhibitor (e.g., pembrolizumab), can have a direct effect on myeloid cells. In some embodiments, an immune checkpoint inhibitor, such as, but not limited to, an anti-PD-1 inhibitor (e.g., pembrolizumab), can act on myeloid cells.

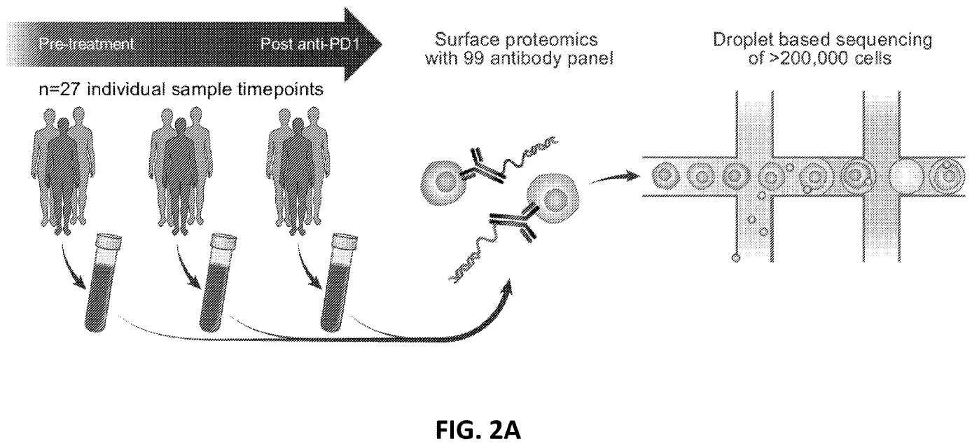

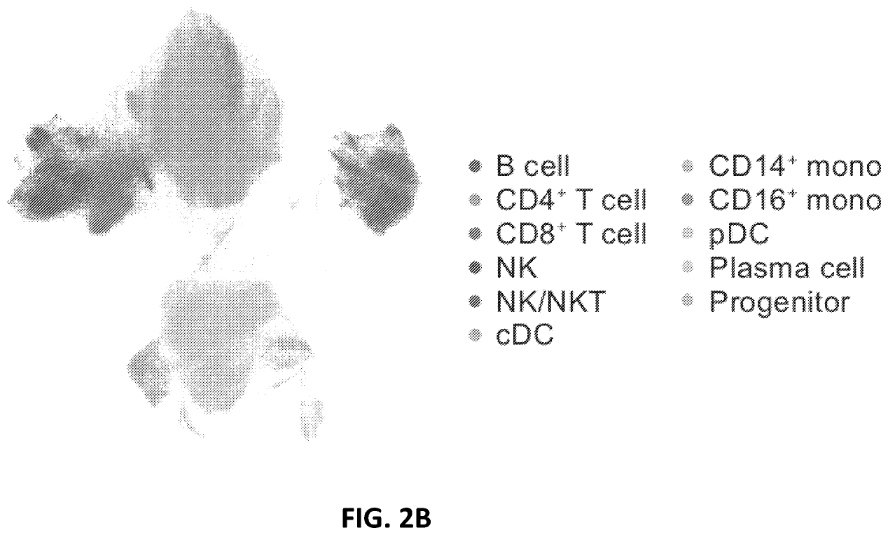

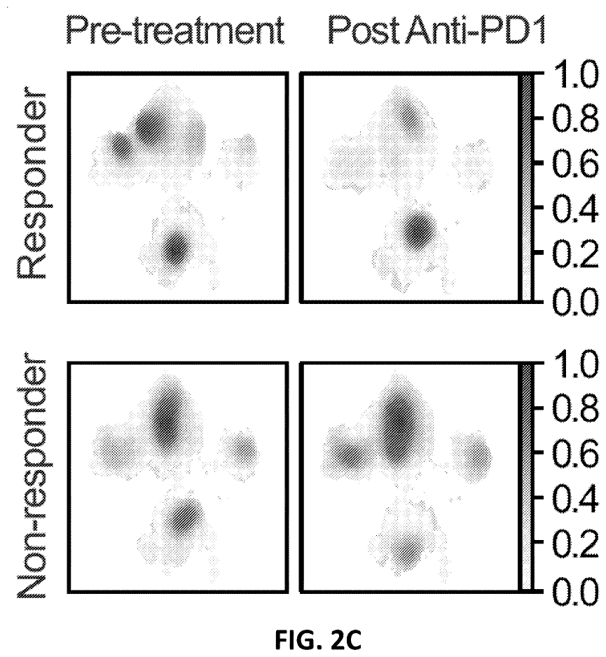

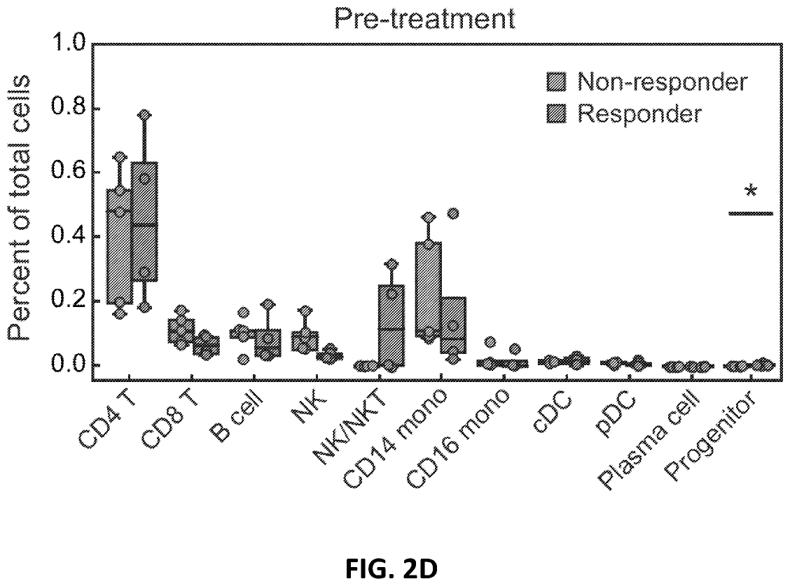

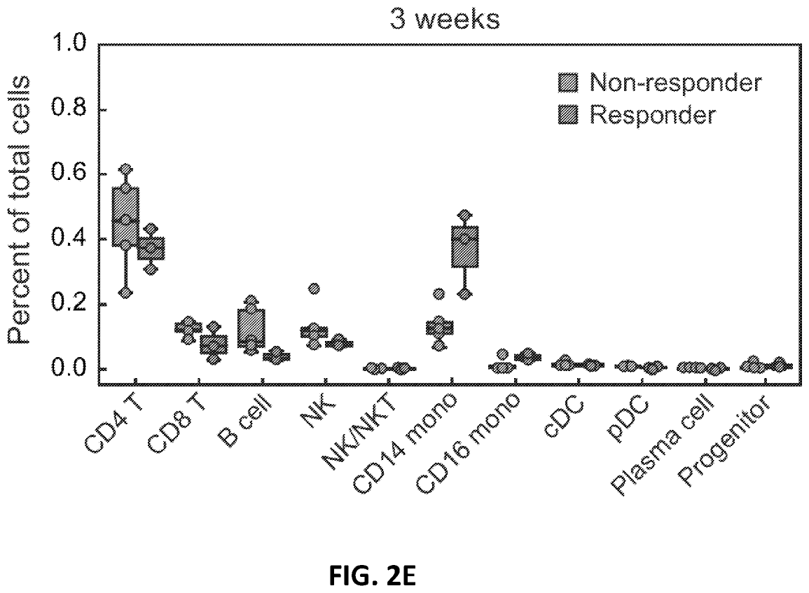

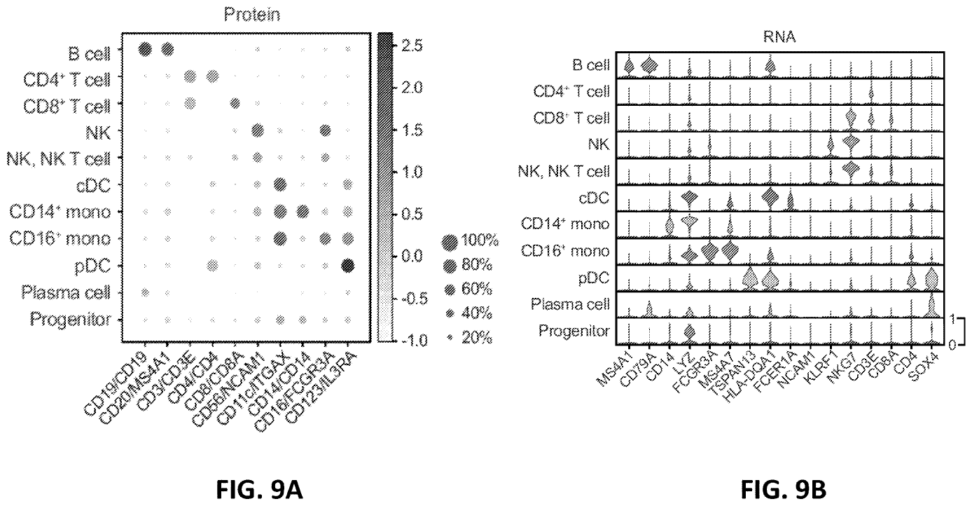

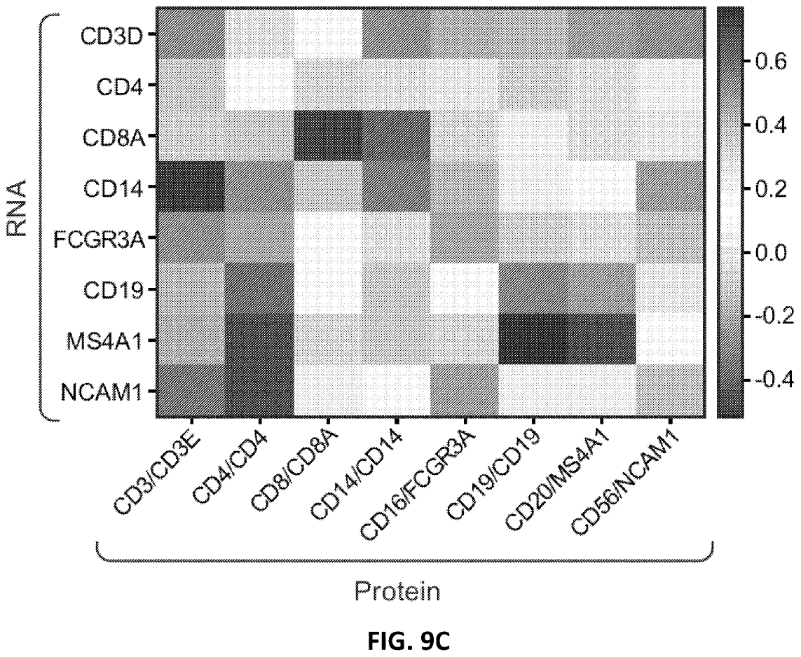

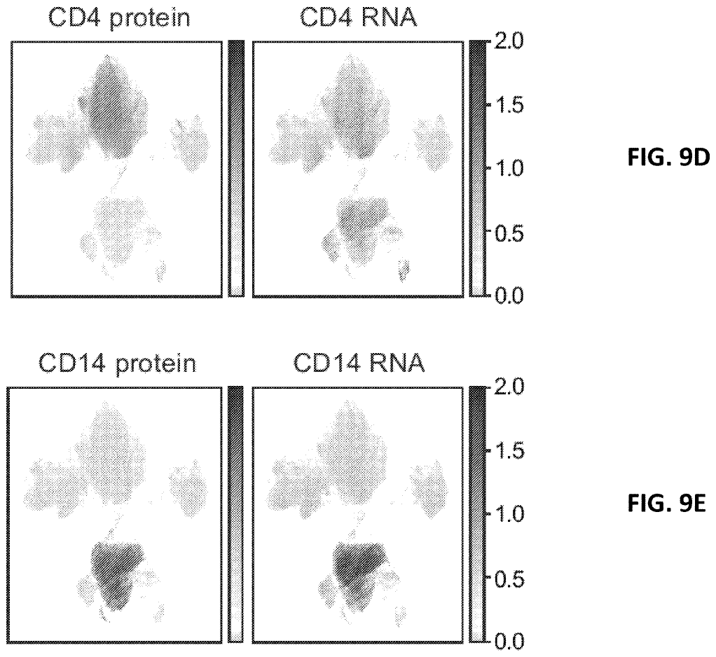

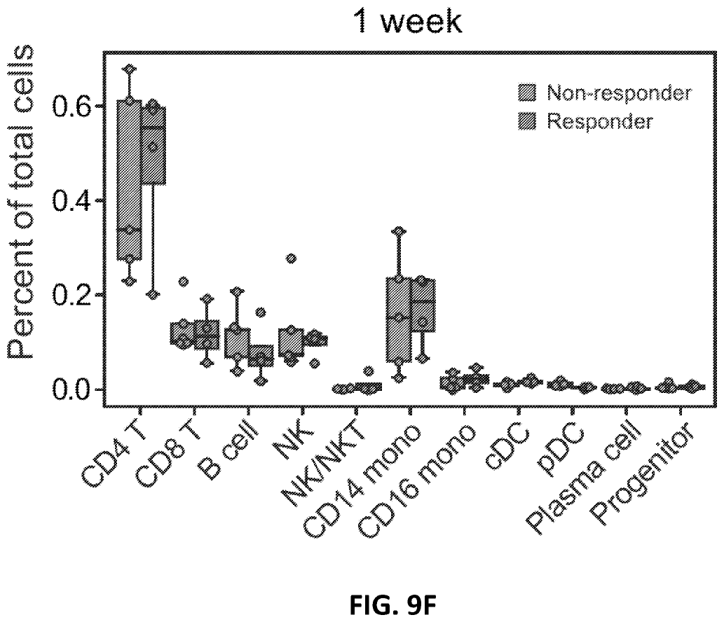

The present disclosure examines circulating immune cells in cancer patients to determine whether there is any differences when analyzed by their clinical outcome to cancer treatment, e.g., with one or more CPIs. In some embodiments, as shown in Example 3, circulating cell composition can be dynamic for both responders (cancer patients whose tumors respond to treatment with an immune checkpoint inhibitor, such as, but not limited to an anti-PD-1) and non-responders (cancer patients whose tumors do not respond to treatment with an immune checkpoint inhibitor, such as, but not limited to an anti-PD-1). In some embodiments, both responders and non-responders can exhibit dynamic changes in the composition within the monocyte compartment. In some embodiments, broad cell frequencies can be not significantly different between responders and non-responders prior to or with treatment with an immune checkpoint inhibitor (FIGS. 2D-2E and 9F). In some embodiments, an increased frequency of circulating CD14⁺ monocytes can be observed in responders prior to treatment with an immune checkpoint inhibitor.

### Circulating Myeloid Sub-Populations

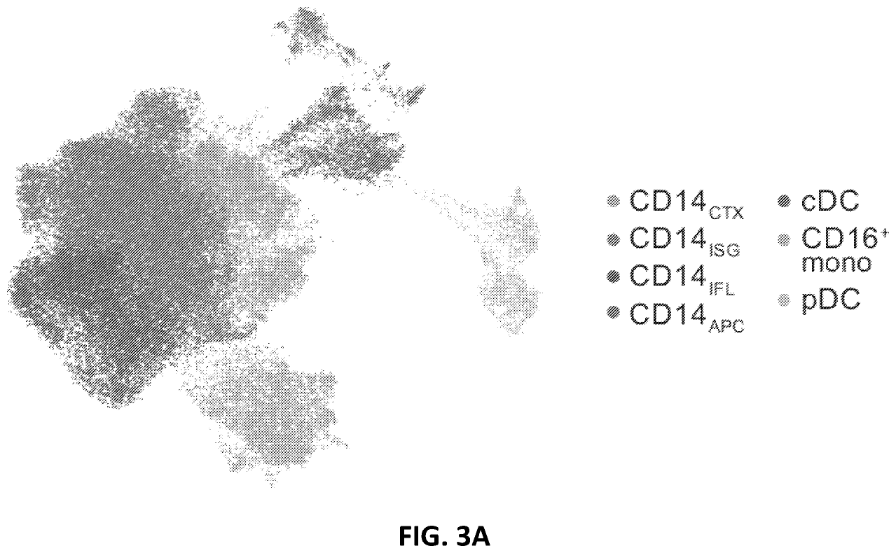

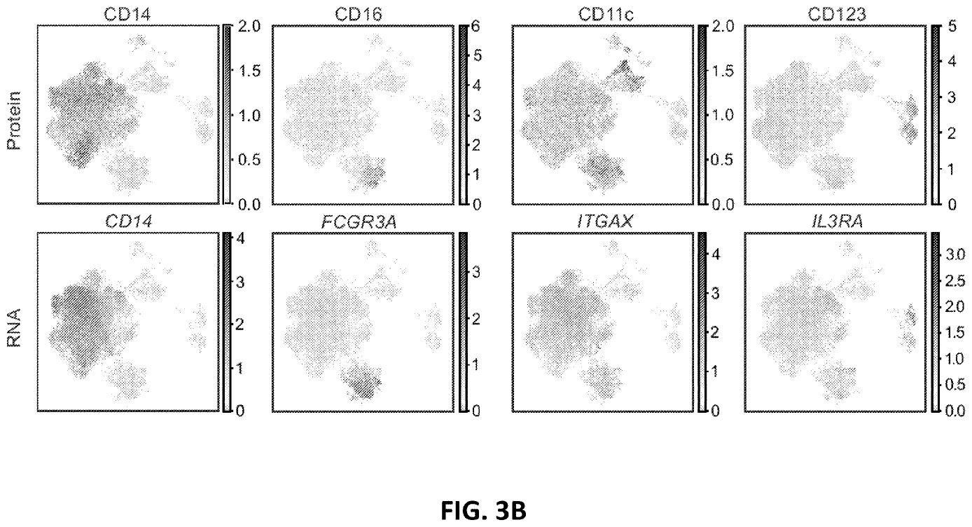

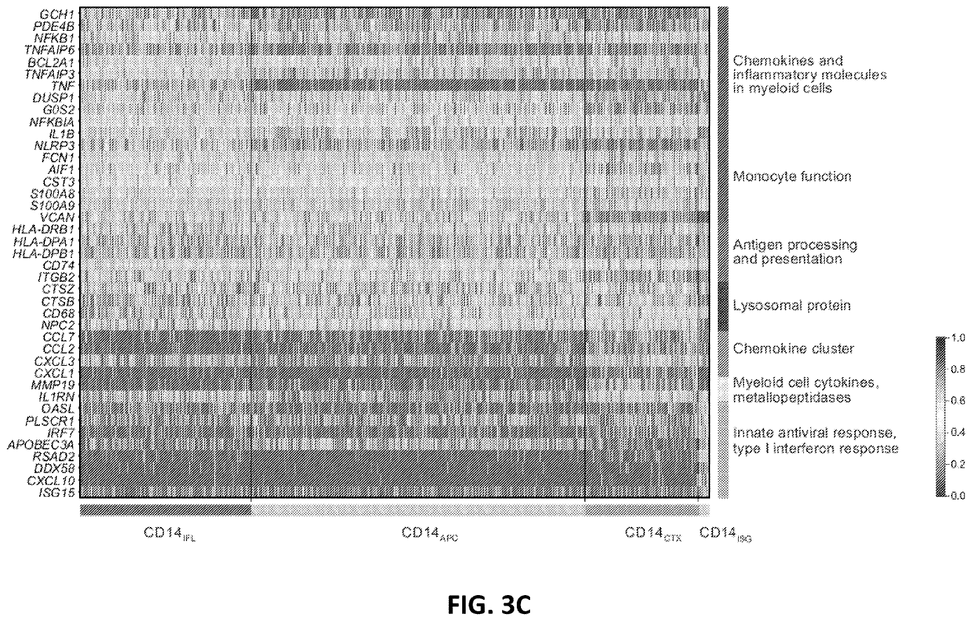

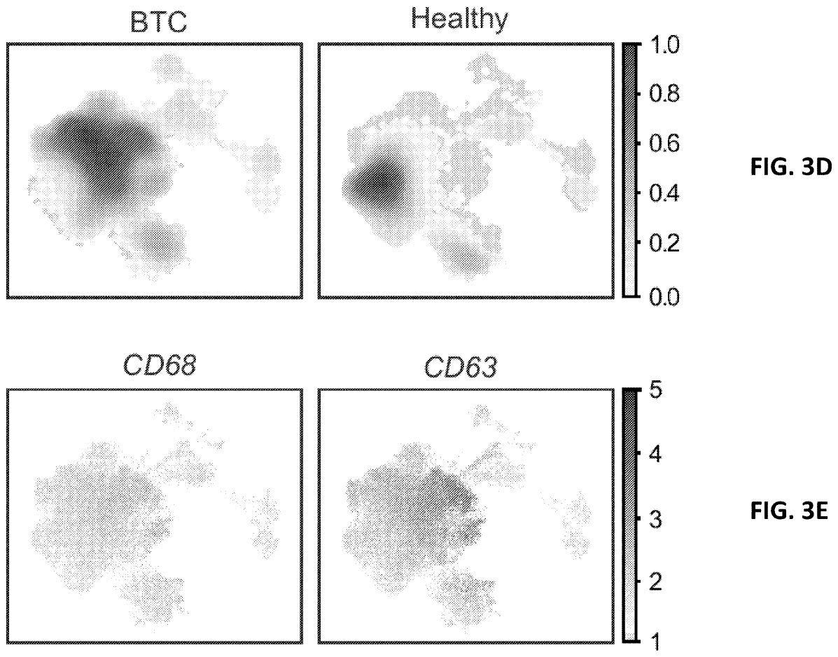

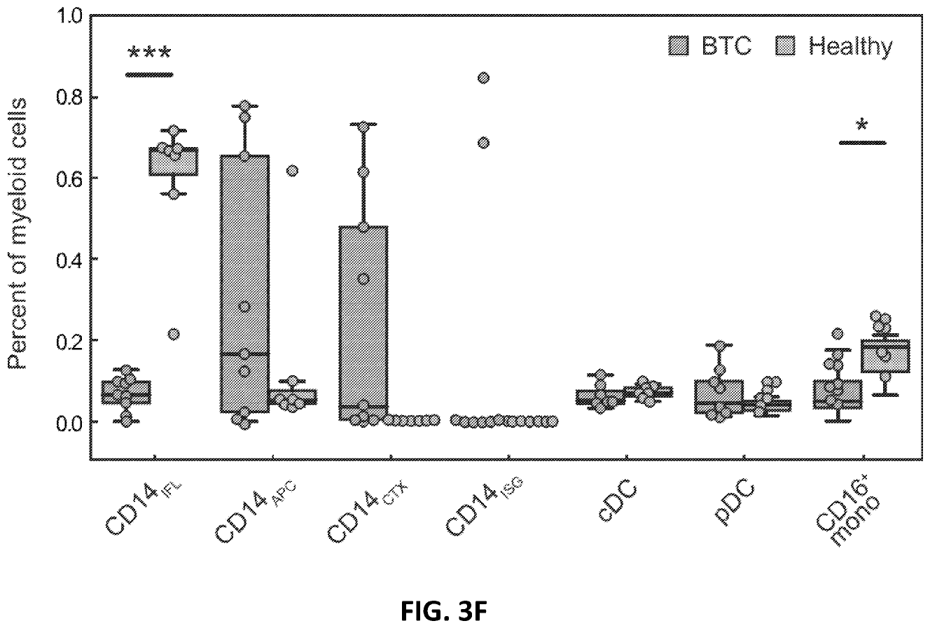

The present disclosure identifies seven sub-populations of the myeloid compartment, focusing on analysis of monocytes and dendritic cells found in PBMCs isolated from one or more subjects having or suspected of having cancer prior to or after receiving treatment of one or more CPIs. As described in Example 4, the seven sub-populations can be identified using, for example, a combination of protein and RNA markers. The identified seven sub-populations include conventional dendritic cells (CDC), plasmacytoid dendritic cells (pDC), CD16⁺ monocytes, and four subpopulations of CD14⁻ monocytes: (1) CD14_{CTX} cells; (2) CD14_{ISG} cells; (3) CD14_{IFL} cells and (4) CD14_{APC} cells. The present disclosure annotates the four subpopulations of CD14⁺ monocytes by canonical immune-specific pathways using gene ontology enrichment analysis of up-regulated genes. Ashburner et al., Nature Genetics 25:25-29 (2000); Li et al., Nat Immunol 15:195-204 (2014). The present disclosure indicates that CD14_{APC}, CD14_{IFL}, and CD14_{ISG} can be canonical CD14⁺ monocytes while CD14_{CTX} can exist on the spectrum of monocytes-macrophages. In some embodiments, the distribution of CD14⁺ sub-populations (CD14_{CTX} cells, CD14_{ISG} cells, CD14_{IFL} cells, and CD14_{APC} cells) can vary between cancer patients and cancer-free subjects with quantitative differences despite there not being an apparent difference when comparing total CD14⁺ monocyte frequencies overall. In comparison to cancer-free subjects, cancer patients prior to treatment can have a decreased frequency of CD14_{IFL} cells and CD16⁺ monocytes and an increased frequency of CD14_{APC} cells. In some embodiments, CD14_{CTX} and CD14_{APC} can be found exclusively in the circulation of cancer patients and not in cancer-free subjects (FIG. 3F).

As described in Example 5 below, myeloid sub-population frequencies and gene signatures can differ by clinical outcome. For example, responders can have a markedly higher frequency of CD14_{APC} cells, while non-responders can have an increased frequency of CD14_{CTX}, pDC, and cDC. In some embodiments, the myeloid sub-populations described herein can represent states of monocyte-macrophage differentiation.

### CD14⁺ Monocyte Sub-populations

A CD14⁺ monocyte subpopulation CD14_{CTX} cells are CD14⁺ monocytes enriched with chemotaxis molecules (e.g., chemokines, chemokine receptors, and pro-inflammatory cytokines). In some embodiments, the chemotaxis molecules enriched in CD14_{CTX} cells can be expressed from one or more genes selected from the group consisting of: ANTXR2, ANXA1, ANXA5, AQP9, ASPH, BASP1, BRI3, CCL2, CCL3, CCL7, CD53, CD68, CTSB, CTSZ, CXCLI, CXCL3, CYPIBI, EMPI, EREG, FCERIG, FLNA, GLIPRI, HLA-A, HLA-B, HLA-C, HLA-DRA, HLA-E, HMOXI, ILIRI, ILIRN, INHBA, KYNU, LAPTM5, LCP1, LGALSI, LGALS3, LHFPL2, MMP19, NINJI, NPC2, NRIP3, PLAUR, PPP1R15A, S100A11, SERPINB2, SERPINB9, SLAMF7, SLC6A6, SLC7A11, SOD2, TGFBI, THBSI, TIMP1, TNFAIP8, and TPM4. CD14_{CTX} can also be distinguished by an increased expression of CD63 and/or CD68 and a lower expression of CD14. In some embodiments, two highly expressed surface markers in CD14_{CTX} can be (1) Tim3 (HAVCR2), an immune checkpoint on T cells that is also expressed by dendritic cells and M2 macrophages; and/or (2) CD29 (ITGB1), an integrin that can mediate chemotaxis and is upregulated in macrophages compared to other myeloid cells.

A CD14⁺ monocyte subpopulation CD14_{APC} cells are CD14⁺ monocytes enriched in monocyte differentiation and function and antigen processing and presentation. In some embodiments, molecules involved in the monocyte differentiation and function and antigen processing and presentation enriched in CD14_{APC} cells can be expressed from one or more genes selected from the group consisting of: MAFB, AIFI, C5ARI, CD14, CD74, CEBPD, CLEC7A, CST3, CTSS, FCNI, GRN, HLA-DPA1, HLA-DPBI, HLA-DRBI, ITGB2, LGALS2, LSTI, LY, LYZ, MAFB, MXD1, PSAP, RPL3, S100A12, S100A8, S100A9, SERPINA1, SLC11A1, THBD, TYROBP, and VCAN.

A CD 14⁺ monocyte subpopulation CD14_{IFL} cells are CD14⁺ monocytes enriched for pathways related to inflammation (e.g., pro-inflammatory cytokines and chemokines, NFκB signaling, and inflammasome function). In some embodiments, molecules in the pathways related to inflammation enriched in CD14_{APC} cells can be expressed from one or more genes selected from the group consisting of: ACSLI, AQP9, ATF3, BCL2A1, BTG2, CCL20, CCL3, CCL3L1, CCL4, CD83, CLEC4E, CLK1, CXCL2, CXCL3, DUSP1, DUSP2, DUSP6, EGR1, EREG, F3, FOS, FOSB, GOS2, GADD45B, GCHI, ICAMI, IER3, IFIT2, ILIA, ILIB, ILIRN, JUN, JUNB, KLF4, KLF6, MAFF, MARCKS, MIR155HG, MNDA, NCF1, NFκB1, NFκBIA, NLRP3, NR4A2, PDE4B, PLAUR, PLEK, PNPLA8, PPPIR15A, PTGS2, PTX3, SGK1, SOD2, STX11, TAGAP, TNF, TNFAIP2, TNFAIP3, TNFAIP6, TNFAIP8, and TRIB1.

A CD 14⁺ monocyte subpopulation CD14_{ISG} cells represents a smaller population of CD14^{low} monocytes with upregulated interferon response genes (ISG) and innate immune signaling. In some embodiments, the upregulated interferon response genes (ISG) and molecules in the innate immune signaling enriched in CD14_{ISG} cells can be expressed from one or more genes selected from the group consisting of: APOBEC3A, AQP9, BCL2A1, C3ARI, CCL3, CCL3L1, CCL4, CCRL2, CD69, CLEC4E, CXCL10, CXCL11, CXCL2, DDX58, DRAMI, HLA-E, IFI6, IFIT2, IFIT3, IL1ORA, ILIB, ILIR1, ILIRN, IL6, INHBA, IRF7, ISG15, ITGB8, LCP2, MIR155HG, MX2, OASL, PLEK, PLSCR1, PTGS2, RIN2, RSAD2, SLAMF7, SOD2, TAGAP, TNF, TNFAIP6, and TRAF1.

The present disclosure identifies cell surface markers and gene signatures of CD14_{CTX} cells that can be used to assess circulating myeloid cells by more conventional means and can be further explored as a circulating biomarker or a target for future treatment(s) for cancer. The present disclosure identifies T cell immunoglobulin and mucin domain-containing protein 3 (Tim3) and CD29 (integrin β1) as more specific combinatorial markers for identifying circulating myeloid cells in patients with cancer (e.g., biliary tract cancer, prostate cancer, and colon cancer). Furthermore, as described herein, CD14_{CTX} cells can express certain molecules associated with immunosuppression such as CXCL8, TGFβ1, and IL-6, which can be targeted when treating cancer. In addition, CD14_{CTX} cells can align with secreted phosphoprotein 1 (SPP1)-expressing tumor-associated macrophages (TAMs). SPP1 (also known as osteopontin (OPN), bone sialoprotein 1 (BSP-1 or BNSP), early T-lymphocyte activation (ETA-1), and 2ar and Rickettsia resistance (Ric)) can be a broadly expressed, pleiotropic molecule, involved in chemotaxis, anti-apoptosis, and maladaptive wound-healing response, with both pro-inflammatory and anti-inflammatory roles. SPP1 expression can correlate with poor prognosis in many cancer types, including biliary cancer, and SPP1⁺ TAMs (Mac_{SPP1}) have been identified in many immune checkpoint inhibitor treatment insensitive cancers, such as colorectal cancer.

In some embodiments, CD14_{CTX} can have increased expressions of several tumor-associated macrophage (TAM) and/or myeloid-derived suppressor cell (MDSC)-related cytokines, such as, but not limited to, IL6, TGFβ1, and CXCL8, when compared to CD14_{APC} cells. In some embodiments, CD14crx can lack expressions of other MDSC-associated genes, such as, but not limited to, ARG1, VEGFA, and IDO1. Although antigen processing and presentation pathways can be enriched in both CD14⁺ monocyte sub-populations CD14_{CTX} and CD14_{APC}, the individual genes and pathways can differ. Exemplary genes that are differently expressed in CD14_{CTX} compared to CD14_{APC} can include, but are not limited to, FTH1, CXCL8, VIM, FTL, MALATI, SERPINB2, CCL3, ILIB, S100A10, EIF1, ANXA1, CXCL3, TPT1, CTSL, THBS1, TIMP 1, CCL4, SOD2, S100A11, and ANXA5. In some embodiments, CD14_{CTX} can express COX2 (PTSG2) and HLA molecules, while CD14_{APC} can express genes related to monocyte surface phenotype (S100A8, S100A9, CD14, FCN1) and function (i.e., the inflammasome-related gene, NLRP3). CD14_{CTX} can also express a distinct set of chemokines involved in the recruitment of CCR2⁺ inflammatory monocytes, a population associated with poor outcomes in cancer patients (e.g., CCL2, CCL7), recruitment of neutrophils (e.g., CXCL1, CXCL2, CXCL3), and associated with T cell exhaustion (e.g., CCL20), pro-inflammatory cytokines (e.g., ILIA, IL1B), as well as molecules associated with cell migration and extracellular matrix digestion (e.g., TIMP1, CTSB, CTSZ).

The present disclosure provides cell surface markers that can distinguish of CD14_{CTX} from other monocyte subpopulations. In some embodiments, the cell surface marker can include T cell immunoglobulin and mucin domain-containing protein 3 (Tim3), an immune checkpoint on T cells that is also expressed by dendritic cells and M2 macrophages. In some embodiments, the cell surface marker can include CD29 (integrin β1), an integrin that can mediate chemotaxis and is upregulated in macrophages compared to other myeloid cells. In some embodiments, high expression of Tim3 and CD29 combination can specifically distinguish CD14_{CTX} from other CD14⁺ monocyte subpopulations. In some embodiments, a cancer patient can have an increased frequency of CD29⁺ Tim3⁺CD68⁺ cells as well as Tim3⁺CD68⁺ and CD29⁺CD68⁺ cells compared to cancer-free subjects. In some embodiments, enrichment of CD29 Tim3⁺ monocytes can be specific to cancer patients, while the frequency of total CD14⁺ or CD68⁺ myeloid cells may not differ significantly between cancer-free subjects and cancer patients.

CD4⁺ and CD8⁺ T Cell Sub-Populations

The present disclosure provides nine unique CD4⁺ and CD8⁺ T cell sub-populations, identified using both transcript and protein markers (Example 8). Six CD4⁺ T cell clusters can include (1) CD4naive naïve and effector memory cells; (2) CD4EM naïve and effector memory cells; (3) CD4Treg FOXP3⁺ regulatory cells; (4) CD4TCF7 cells characterized by high expression of TCF7; (5) CD4_{SOCS3} cells characterized by high expression of SOCS3; and (6) CD4_{ISG} cells characterized by high expression of ISG. Three clusters of CD8⁺ T cells can include (7) CD8naive naïve cells; (8) CD8GrB effector cells expressing predominantly GZMB/GZMH; and (9) CD8GrK effector cells expressing predominantly GZMK.

The present disclosure provides that frequency of CD14_{CTX} cells can be correlated with suppressor of cytokine signaling 3 (SOCS3) expression in CD4⁺ T cells (CD4_{SOCS3} cells). SOCS3 is a known negative regulator of cytokine signaling and a mediator of T cell immune paralysis. T cell unresponsiveness induced in T cells by cancer-associated myeloid cells is an emerging mechanism of immunosuppression distinct from those mediated by other immune checkpoint pathways. Circulating CD4_{SOCS3} cells can also exhibit immune paralysis following stimulation in vitro (Example 9).

In some embodiments, CD14_{CTX} cells isolated from cancer patients' circulation can suppress proliferation of CD4⁺ T cells. Further, consistent with the association between the frequencies of CD14_{CTX} cells with CD4_{SOCS3} cells, CD14_{CTX} cells can induce SOCS3 expression in sorted naïve CD4″ T cells. In some embodiments, SOCS3 expression can be associated with immune paralysis in CD4⁺ T cells in the setting of cytokine exposure. In some embodiments, CD4_{SOCS3} cells from cancer patients can retain the ability to produce IFNγ, TNFα, and IL2. In contrast, CD4_{SOCS3} cells can fail to produce these cytokines in response to stimulation. In some embodiments, CD14_{CTX} cells and CD4_{SOCS3} cells interact within tumor microenvironment.

The present disclosure provides unexpected and surprising frequency associations between myeloid sub-populations and T cell sub-populations. In some embodiments, frequency of CD14_{CTX} cells can be positively correlated with the frequency of CD4_{SOCS3} and negatively correlated with CD4TCF7 frequency. In some embodiments, frequency of CD14_{APC} can be positively correlated with the frequency of CD4TCF7 and not correlated with frequency of CD4_{SOCS3}. As described in Example 8, the positive correlation of CD4TCF7 with CD14_{APC} and negative correlation with CD14_{CTX} in biliary tract cancers patients is an unexpected finding because TCF7 expression within CD4⁺ T cells is associated with the capability to self-renew. Also, SOCS3 is a negative regulator of cytokine signaling and is associated with T cell dysfunction.

### Tumor-Associated Macrophages (TAMs)

As demonstrated herein, the monocyte subpopulation CD14_{CTX}, which can be associated with treatment insensitivity to one or more CPIs, such as an anti-PD-1 (e.g., pembrolizumab), has increased expression of chemokines and molecules involved in extracellular matrix digestion, which can facilitate migration into the tumor microenvironment and can represent a precursor of TAMs. This is further supported by overall highly correlated gene signatures, with downregulation of genes related to extravasation, in TAMs from primary tumor tissue samples (e.g., cholangiocarcinoma tissue samples). Incongruous findings have been observed regarding the association of TAMs with biliary cancer patient prognosis, highlighting the challenge in applying one label to a heterogeneous group of cells that can have anti-oncogenic or pro-oncogenic phenotypes. The present disclosure demonstrates that alteration in monocytes are associated with clinical response to treatment with one or more CPIs, such as a PD-1 inhibitor (e.g., pembrolizumab). This aligns with findings in melanoma patients, although clinical associations with circulating monocyte populations emerges on treatment rather than being present at baseline.

The present disclosure provides that CD14_{CTX} gene signature can be correlated with SPP1⁺ tumor-associated macrophages (TAMs) (i.e., Mac_{SPP1} cells) in the tumor microenvironment. Thus, CD14_{CTX} gene signature can be associated with poor prognosis in cancer patients with immune checkpoint inhibitor insensitive tumors (Example 7). CD14_{CTX} cells can express chemokine receptors that might facilitate migration into tumor tissues. Tumor-associated myeloid cells (TAMs) can consist of dendritic cells, neutrophils, macrophages with high APOE expression (Mac_{APOE}), macrophages with high SPP1 expression (Mac_{SPP1}), CD14⁺ monocytes, CD16⁺ monocytes, and intermediate CD14⁺CD16″ monocytes. Mac_{SPP1} as used herein refers to tumor-associated macrophages (TAMs) with high SPP1 expression. Among the tissue-associated myeloid populations, the expression profile of CD14_{CTX} cells can be most correlated with Mac_{SPP1}, exemplified by the shared expression of differentially expressed CD14_{CTX} genes including HAVCR2 and ITGB1. In some embodiments, two genes that differs in expression between MAC_{SPP1} and CD14crx can be related to chemotaxis and extravasation (e.g., SERPINB2, TIMP1). In some embodiments, SPP1⁺HAVCR2 CD68⁺ myeloid cells can be detected within tumor tissues from on-treatment biopsies. Accordingly, the present disclosure provides that the existence of a TAM population in tumor tissues can be analogous to a high CD14_{CTX} sub-population in circulating myeloid cells.

The CD14_{CTX} gene signature (i.e., an increased CD14_{CTX} sub-population) presently described herein can be applied to any type of cancer that is insensitive treatment with one or more CPIs, such as an anti-PD-1 (e.g., pembrolizumab) (Example 7). In some embodiments, high expression of the CD14_{CTX} gene signature can be associated with a significantly worse overall survival.

### Combination Therapies

The present disclosure contemplates the use of one or more CPIs alone or in combination with one or more therapeutic agents that is not a CPI. The one or more therapeutic agents can be small chemical molecules; macromolecules, such as proteins, antibodies, peptibodies, peptides, DNA, RNA or fragments of such macromolecules; or cellular or gene therapies. The combination therapy can target different, but complementary, mechanisms of action and thereby have a synergistic therapeutic or prophylactic effect on the underlying disease, disorder, or condition. In addition, or alternatively, the combination therapy can allow for a dose reduction of the one or more CPIs, thereby ameliorating, reducing or eliminating adverse effects associated with the one or more CPIs.

The one or more therapeutic agents in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a subject's course of therapy. If the combination is formulated as separate compositions, in some embodiments, the separate compositions can be provided together in a kit.

In some embodiments, the one or more immune checkpoint inhibitor can be administered or applied sequentially to the one or more therapeutic agents, e.g., where the one or more of therapeutic agents is administered prior to or after the administration of the immune checkpoint inhibitor according to this disclosure. In other embodiments, the immune checkpoint inhibitor can be administered simultaneously with one or more of the therapeutic agents, e.g., where the immune checkpoint inhibitor is administered at or about the same time as one or more of the therapeutic agents; the immune checkpoint inhibitor and one or more of the therapeutic agents can be present in two or more separate formulations or combined into a single formulation (i.e., a co-formulation). Regardless of whether the therapeutic agent(s) are administered sequentially or simultaneously with the immune checkpoint inhibitor, they are considered to be administered in combination for purposes of the present disclosure.

The immune checkpoint inhibitor of the present disclosure can be used in combination with the one or more therapeutic agents in any manner appropriate under the circumstances. In one embodiment, treatment with the one or more therapeutic agents and the one or more CPIs can be maintained over a period of time. In another embodiment, treatment with the one or more therapeutic agents can be reduced or discontinued (e.g., when the subject is stable), while treatment with the one or more CPIs can be maintained at a constant dosing regimen. In a further embodiment, treatment with the one or more therapeutic agents can be reduced or discontinued (e.g., when the subject is stable), while treatment with the one or more CPIs can be reduced (e.g., lower dose, less frequent dosing or shorter treatment regimen). In yet another embodiment, treatment with the one or more therapeutic agents can be reduced or discontinued (e.g., when the subject is stable), and treatment with the one or more CPIs can be increased (e.g., higher dose, more frequent dosing or longer treatment regimen). In yet another embodiment, treatment with the one or more therapeutic agents can be maintained and treatment the one or more CPIs can be reduced or discontinued (e.g., lower dose, less frequent dosing or shorter treatment regimen). In yet another embodiment, treatment with the one or more therapeutic agents and treatment with the one or more CPIs can be reduced or discontinued (e.g., lower dose, less frequent dosing or shorter treatment regimen).

The one or more CPIs can be administered with vaccines eliciting an immune response against a cancer. Such immune response can be enhanced by the one or more CPIs. The vaccine can include an antigen expressed on the surface of the cancerous cell and/or tumor of a fragment thereof effective to induce an immune response, optionally linked to a carrier molecule.

A CPI treatment with the one or more CPIs can be combined with other treatments effective against the disorder being treated. When used in treating a proliferative condition, cancer, tumor, or precancerous disease, disorder or condition, the one or more CPIs can be combined with chemotherapy, radiation (e.g., localized radiation therapy or total body radiation therapy), stem cell treatment, surgery or treatment with other biologics.

In some embodiments, the one or more therapeutic agents can include one or more chemotherapeutic agents. Non-limiting examples of a chemotherapeutic agent can include alkylating agents, such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, such as carmustine, chlorozotocin, fotemustine, lomustine, nimustine, ranimustine; antibiotics, such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, pomalidomide potfiromycin, puromycin, quelamycin, rodorubicin, streptonigrin, streptozocin, tubercidin, ubenimex, zinostatin, zorubicin; anti-metabolites, such as methotrexate and 5-fluorouracil (5-FU); folic acid analogs, such as denopterin, methotrexate, pteropterin, trimetrexate; purine analogs, such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens, such as calusterone, dromostanolone propionate, epitiostanol, mepitiostane, testolactone; anti-adrenals, such as aminoglutethimide, mitotane, trilostane; folic acid replenisher, such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel, and docetaxel; chlorambucil; gemcitabine; 6-thioguanine; mercaptopurine; methotrexate; platinum and platinum coordination complexes, such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; anthracyclines; and pharmaceutically acceptable salts, acids, or derivatives of any of the above.

In some embodiments, a combination therapy includes the one or more CPIs and an antibody directed at a surface antigen preferentially expressed on the cancer cells relative to control normal tissue. Some examples of the antibody that can be administered in combination therapy with the one or more CPIs for treatment of cancer can include trastuzumab (e.g., Herceptin®) against the HER2 antigen, bevacizumab (e.g., Avastin®) against VEGF, or antibodies to the EGF receptor, such as cetuximab (Erbitux®) and panitumumab (Vectibix®).

Other therapeutic agents that can be administered with the one or more CPIs can include antibodies or other inhibitors of any of PD-1, PD-L1, CTLA-4, 4-1BB (CD137), TIGIT, B and T lymphocyte attenuator (BTLA), poliovirus receptor-related immunoglobulin domain-containing (PVRIG), V-domain Ig suppressor of T cell activation (VISTA), T-cell immunoglobulin mucin-3 (TIM-3), and lymphocyte activation gene 3 (LAG-3); or other downstream signaling inhibitors, e.g., mTOR and GSK3ß inhibitors; and cytokines, e.g., interferon-γ, IL-2, and IL-15. The choice of the antibody or other therapeutic agents for combination therapy depends on the cancer being treated. Optionally, the cancer can be tested for expression or preferential expression of an antigen to guide selection of an appropriate antibody or other inhibitors.

### Pharmaceutical Compositions

Pharmaceutical compositions of one or more CPIs with an optional therapeutic agent(s) for parenteral administration can be sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the one or more CPIs can be formulated in aqueous solutions, such as in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, the one or more CPIs can be in lyophilized form for constitution with a suitable vehicle, e.g., sterile pyrogen-free water, before use. The concentration of the one or more CPIs in liquid formulations can vary. An appropriate concentration in any given instance can be ascertained by those skilled in the art.

### EXAMPLES

These examples are provided for illustrative purposes only and not to limit the scope of the claims provided herein.

### Example 1. Experimental Protocols

This Example describes experimental methods followed in Examples 2-9.

Patient Samples: Peripheral blood mononuclear cells (PBMCs) were obtained from patients pre- and on-treatment (per UCSF institutional review board (IRB) #15-18420) from the clinical trial of staggered or simultaneous GM-CSF and anti-PD-1 (pembrolizumab) (ClinicalTrials.gov identifier: NCT02703714). Patients, per eligibility criteria, had advanced biliary tract cancer previously treated with chemotherapy, and no active uncontrolled infections. biliary tract cancer (BTC) patients started treatment with anti-PD-1 (administered intravenously starting on cycle 1 day 1 (C1D1) and repeating every 3 weeks) and subsequently received GM-CSF (administered subcutaneously in cycles 2 and 3 for 14 days each). Blood samples from BTC patients were profiled from baseline, 1 week following anti-PD-1 treatment, and 3 weeks following anti-PD-1 treatment immediately prior to cycle 2; the studies provided herein did not examine effects of GM-CSF, as patients received GM-CSF after the collection of these sample time-points. Responders (n=4) were characterized as patients that had an objective partial response or stable disease by imaging, resulting progression-free survival for 6 months or longer. Non-responders (n=5) were patients that did not have objective tumor responses and/or who had progression-free survival less than 6 months. Tumor samples were collected from patients biopsied as part of the Phase II clinical trial and from patients undergoing standard-of-care resections and consented under the UCSF Hepatobiliary Tissue Bank and Registry (IRB #12-09576). Cancer-free subject PBMCs were collected from age and gender-matched healthy donors as part of the Cancer Immunotherapy Biobanking protocol and the Immune Cell Census (IRB #15-16385 and #19-27147, respectively); cancer-free subject samples reflect one time-point, with multiple independent replicates sequenced. Informed consent was obtained from all patients for participation in the listed trials and for use of blood and tumor samples in research studies.

Processing of Samples, Single Cell RNA Sequencing, and CITEseg: Blood samples were processed using ficoll (Amersham); after centrifugation, the peripheral blood mononuclear cell (PBMC) layer was isolated and cryopreserved in cell media with human serum and DMSO. Previously frozen PBMCs from cancer-free subjects and BTC patients were thawed using media containing RPMI, heat-inactivated sterile filtered human serum, penicillin-streptomycin, non-essential amino acids, sodium pyruvate, and L-glutamine (CHM media). Samples were then incubated for DNAse I before washing and counting. 1×10⁶ cells from 16 unique samples were combined and stained with one pooled cocktail containing 99 AbSeq antibody-oligonucleotide conjugates (Table 1) per standard protocols, following pre-incubation with TruStain FcX (Fc Receptor Blocking Solution, Biolegend). (Olvera et al., Protein and transcriptome quantitation using BD AbSeq™ Antibody-Oligonucleotide. technology and the 10X Genomics Chromium™ Single Cell Gene Expression Solution, Department of Medicine, University of California San Diego, 2018). Samples from different individuals and time-points were randomly mixed across experiments to minimize batch and confounding effects. Droplet-based single cell RNA sequencing (scRNAseq) was performed using the 10× Genomics Chromium Single Cell 3′ Reagent Kits v3, according to manufacturer instructions. For tumor tissues, samples were digested in RPMI containing Collagenase I & II and DNAse I, minced, and digested for one hour using the GentleMACS system (Miltenyi Biotec). Isolation of live cells was performed using MACS LS columns (Miltenyi Biotec). scRNAseq of tumor samples was completed on fresh material with 10× Genomics 5′ version 1 kits. All sequencing was performed on an Illumina NovaSeq S4 sequencer with paired end 200 base pair read length and 25,000 reads per droplet.

TABLE 1
Antibodies and clones used in CITE-seq

| Antibody | Clone | Antibody | Clone | Antibody | Clone |
|---|---|---|---|---|---|
| B7-H4 | MIH43 | CD2 | RPA-2.10 | CD45RA | HI100 |
| CD1a | HI149 | CD20 | 2H7 | CD45RO | UCHL1 |
| CD1c | F10/21A3 | CD206 | 19.2 | CD49a | SR84 |
| CD10 | HI10a | CD21 | B-ly4 | CD49b | 12F1 |
| CD103 | Ber-ACT8 | CD226 | DX11 | CD49d | 9F10 |
| CD117 | YB5.B8 | CD235a | GA-R2 (HIR2) | CD49e | IIA1 |
| CD11a | HI111 | CD24 | ML5 | CD5 | UCHT2 |
| CD11b | M1/70 | CD25 (IL-2R) | 2A3 | CD54 | HA58 |
| CD11c | B-ly6 | CD26 | M-A261 | CD56 | NCAM16.2 |
| CD123 | 7G3 | CD27 | M-T271 | CD61 | VI-PL2 |
| CD124 | hIL4R-M57 | CD270 | CW10 | CD62L | DREG-56 |
| CD126 | M5 | CD272 | J168-540 | CD69 | FN50 |
| CD127 | HIL-7R- | CD273 | MIH18 | CD7 | M-T701 |
| | M21 | | | | |
| CD13 | WM15 | CD274 | MIH1 | CD8 | RPA-T8 |
| | | (PD1L) | | | |
| CD133 | W6B3C1 | CD275 | 2D3/B7-H2 | CD80 | L307.4 |
| | | (ICOSL) | | | |
| CD134 | ACT35 | CD278 (ICOS) | DX29 | CD81 | JS-81 |
| CD137 | 4B4-1 | CD279 (PD-1) | EH12.1 | CD86 | 2331 (FUN-1) |
| CD14 | MφP9 | CD28 | CD28.2 | CD9 | M-L13 |
| CD141 | 1A4 | CD29 | MAR4 | CD90 | 5E10 |
| CD152 (CTLA4) | BNI3 | CD3 | SK7 | CD94 | HP-3D9 |
| CD154 (CD40L) | TRAP1 | CD30 | BerH8 | CD95 | DX2 |
| CD155 | TX24 | CD314 | 1D11 | CD98 | UM7F8 |
| CD16 | 3G8 | CD32 | FLI8.26 | CXCR5 | RF8B2 |
| | | | | (CD185) | |
| CD163 | GHI/61 | CD326 | EBA-1 | GITR | V27-580 |
| | | | | (CD357) | |
| CD178 | NOK-1 | CD33 | WM53 | HLA-ABC | G46-2.6 |
| CD18 | 6.7 | CD335 | 9E2/NKp46 | IgD | IA6-2 |
| | | (NKp46) | | | |
| CD183 (CXCR3) | 1C6/CXCR3 | CD34 | 581 | IgG | G18-145 |
| CD184 | 12G5 | CD38 | HIT2 | IL21R | 17A12 |
| | | | | (CD360) | |
| CD19 | SJ25C1 | CD39 | TU66 | LAG3 | T47-530 |
| CD194 (CCR4) | 1G1 | CD4 | SK3 | NKp44 | p44-8 |
| | | | | (CD336) | |
| CD195 (CCR5) | 2D7/CCR5 | CD40 | 5C3 | TCRab | IP26 |
| CD196 (CCR6) | 11A9 | CD44 | G44-26 | TCRgd | B1 |
| CD197 (CCR7) | 3D12 | CD45 | HI30 | TIM-3 | 7D3 |

RNA Extraction and bulk RNA Sequencing: The RNeasy Mini Kit (Qiagen) was used to extract RNA from minimum 2.5×10⁵ cells per PBMC sample. cDNA was prepared using methods previously described, with the Smart-seq2 protocol, and libraries were prepared using Nextera XT DNA Sample Preparation Kit. Bulk RNA from each sample was sequenced at a depth of at least 2×10⁷ reads per cell on the Illumina Novaseq S4 and aligned to human genome build 38 with STAR. Pre-processing of aligned sequencing data and identification of single nucleotide polymorphisms was performed using the Genome Analysis Toolkit. Demuxlet (https://github.com/statgen/demuxlet) was used for sample deconvolution for biliary cancer multiplexed PBMC samples, removing any samples that lacked high confidence in sample identification.

Pre-Processing of scRNAseg Data: CellRanger version 3.1.0 (10× Genomics, Genome Build: GRCh38 3.0.0) was used to align the raw sequencing data. The ADT library sequences were aligned to a customized reference genome provided by BD containing the oligonucleotide sequences corresponding to each antibody. The SCANPY data analysis pipeline was used for pre-processing and analysis of scRNAseq data, with the following software versions: scanpy 1.4.6, anndata 0.7.1, umap 0.4.1, numpy 1.18.1, scipy 1.4.1, pandas 1.0.3, scikit-learn 0.21.2, statsmodels 0.10.1, python-igraph 0.8.0, and louvain 0.6.1. The following cutoffs were applied for filtering high quality cells: <20% mitochondrial genes, >100 and <2500 genes expressed per cell, and excluded platelets, red blood cells, and doublets. Ribosomal genes and genes detected in less than three cells were filtered out. Following sequencing alignment, pre-processing, quality control, and doublet removal, over 230,000 cells were recovered from all samples combined, corresponding to greater than 5,000 cells per sample. The data were log 2 plus one transformed, normalized to 10,000 counts per cell, regressed out gender, percent mitochondrial genes, and number of gene counts, and scaled genes to unit variance. Batch correction was performed using ComBat and highly variable genes present in greater than 4 of 11 independent experiments, using the SCANPY function for highly variable genes, and principle component analysis was ran with SCANPY. K-nearest neighbor graph construction and clustering on gene expression data were performed; for analysis of all immune cells, cells with a resolution of 1.0 were clustered. Myeloid or T cells were re-clustered individually, removing any contaminating cells (non-myeloid or non-T cell). A resolution of 0.3 was used for myeloid; a resolution 0.6 was used for T cells. The protein data was processed by log 2 plus one transformation, regressing out batch, and scaled as for RNA. For the fresh tumor tissue dataset, the same pre-processing pipeline was applied for the fresh tumor tissue dataset. Previously established gene lists were used for the annotation of cells in cholangiocarcinoma, including immune and non-immune cells. Four myeloid clusters, three lymphocyte clusters, and three malignant cell clusters were identified. The intra-tumoral myeloid cells and T cells were independently re-clustered using a resolution of 0.3 and 1, respectively.

scRNAseg Analysis: The SCANPY embedded function was used to determine top differentially expressed genes for all immune cells, T cells and myeloid sub-types; for further analysis, MAST was used (see Statistical Analysis below). Cell types were annotated using commonly expressed protein (FIG. 9A, Table 1) and transcript markers (FIG. 9B). CITE-seq generally produced strong correlation (R=0.40 to 0.71, p=1.25×10⁻⁷ to 9.31×10⁻³, inclusive of all values except for CD4) between protein and RNA expression for canonical immune cell type markers across individual samples, except in genes that had low levels of transcript abundance such as CD4 (FIG. 9C-9E). COMET was used to identify combinatorial gene expression by analyzing a subset of 1000 equally sampled cells from the CD14_{CTX}, CD 14_{APC}, and CD14_{IFL} populations (see Example 4 below) and running three iterations with different random samples. This list was used to identify highly ranked gene pairs that were cell surface proteins contained in the CITEseq panel. Pseudotime analysis was performed using Monocle v2.10.1, using a sub-sample of maximum 10,000 total cells with equal cell number sampled from each cell type. For gene signature comparisons between circulating immune cells and intra-tumoral immune cells, a matrix of pseudobulk expression was created for each cell type and then correlation analysis was performed on pseudobulk gene expression profiles.

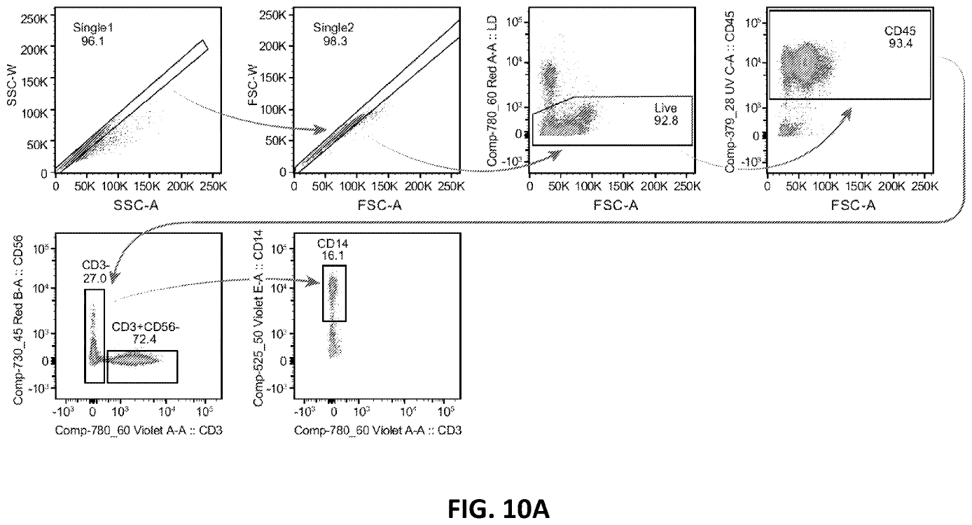

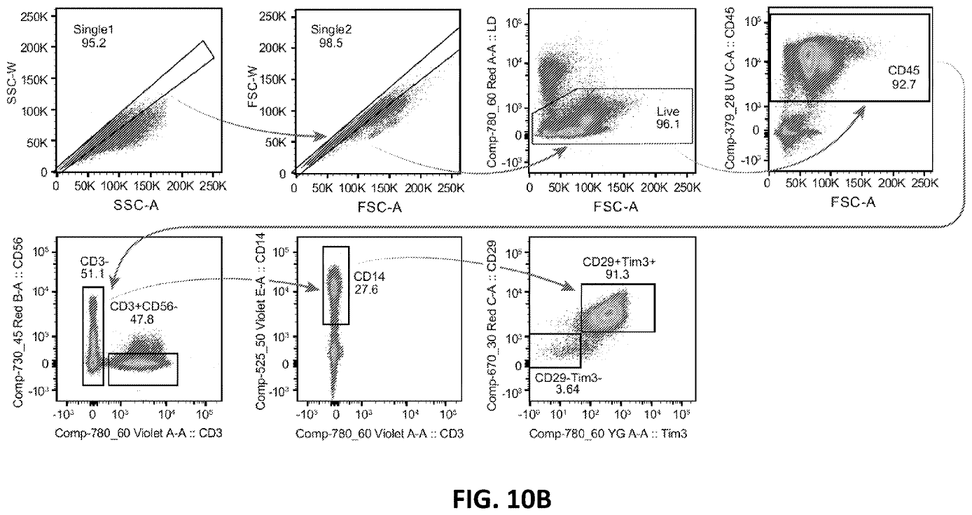

Flow cytometry and in vitro experiments: The PBMC samples were thawed as described for scRNAseq analysis above, incubated with TruStain FcX, and stained with LIVE/DEAD Fixable Near-IR Dead Cell Stain, followed by surface antibody staining. For CD68, SOCS3, and cytokine staining, intracellular staining was performed using the Intracellular Fixation & Permeabilization kit. Data was acquired using the LSRFortessa cytometer. FACS was performed with the gating schema described in below Example sections and FIGS. 10A-10B to obtain the sorted populations from cancer-free subject and cancer patient PBMCs, using a FACSAria Fusion. In T cell/myeloid cell co-cultures, cells were plated at 1:1 ratio for effector T cells: myeloid population, with 1×10⁵ T cells per well, in CHM media and 10 units IL-2. Cells were harvested on day 6 for analysis with flow cytometry. Intracellular SOCS3 staining was performed using an unconjugated primary antibody and a fluorescently-conjugated secondary antibody. For T cell stimulation experiments, anti-CD3/CD28 beads were used in culture for 3 days before harvest; protein transport inhibitor cocktail was added to co-cultures for 4 hours before harvest and intracellular cytokine staining. Complete information for antibodies used is in Table 2. For suppression assays, sorted T cells were stained with CFSE (CellTrace, Invitrogen) per manufacturer instructions prior to co-culture with monocytes.

TABLE 2
Antibodies used in surface and intracellular
staining experiments and in cell sorting

| Fluorophore | Antibody | Manufacturer | Cat no | Clone |
|---|---|---|---|---|
| BV421 | Goat anti- | Jackson | 111-675- | N/A |
| | rabbit IgG | ImmunoResearch | 144 | |
| | (secondary | Laboratories, | | |
| | for SOCS3) | Inc. | | |
| BV510 | CD14 | Biolegend | 367123 | 63D3 |
| BV650 | CD8 | Biolegend | 344729 | T8 |
| BV785 | CD3 | Biolegend | 300472 | UCHT1 |
| PE-Cy7 | Tim3 | BD Biosciences | 345028 | F38-2E2 |
| BUV395 | CD45 | BD Biosciences | 563791 | HI/30 |
| AF647 | CD29 | Biolegend | 303018 | Ts2/16 |
| APC-R700 | CD56 | BD Biosciences | 565139 | NCAM16.2 |
| PE | CD4 | Biolegend | 300508 | RPA-T4 |
| AF488 | CD4 | Biolegend | 317420 | OKT4 |
| BV711 | IL-2 | BD Biosciences | 563946 | 5344.111 |
| PE | TNFa | Biolegend | 502909 | MAb11 |
| AF647 | IFNg | Biolegend | 502516 | 45.B3 |
| N/A | SOCS3 | Cell Signaling | 52113 | D6E1T |
| | | Technology | | |
| BV421 | mouse IgG1, | Biolegend | 400158 | MOPC-21 |
| | k | | | |
| AF647 | mouse IgG1, | Biolegend | 400136 | MOPC-21 |
| | k | | | |
| PE | mouse IgG1, | Biolegend | 400112 | MOPC-21 |
| | k | | | |
| BV711 | mouse IgG1, | Biolegend | 400168 | MOPC-21 |
| | k | | | |

BV = Briliant Violet;
AF = Alexa Fluor;
PE = R-phycoerythrin;
APC = Allophycocyanin;
BUV = Brilliant Ultraviolet

Tissue staining: RNAscope in situ hybridization and immunofluorescence were performed on 4 μm FFPE sections obtained from control tonsil and from biopsies collected from BTC patients treated on the clinical trial. Tissues were pre-treated with target retrieval reagents and protease to improve target recovery based on guidelines provided in the RNAscope Multiplex Fluorescent Reagents Kit v2 Assay protocol. mRNA expression was demonstrated using probes for CD68, SOCS3, SPP1, and HAVCR2 (Table 3). Probes were hybridized with Opal 7-Color Manual IHC Kit (PerkinElmer) to produce discrete points of light. Samples were then stained for CD4 and CD3 and with the secondary antibodies given in Table 2. Tissues were counterstained with DAPI. Slides were imaged using TCS SP8 X white light laser inverted confocal microscope.

TABLE 3
RNAscope probe and antibody information

| | | | | | | | | | Catalog # | |
|---|---|---|---|---|---|---|---|---|---|---|
| | | | Catalog | | Dilution | | | | (secondary | Secondary |
| Target | Type | Manufacturer | # | Isotype | used | Time | Detection | Manufacturer | antibody) | dilution |
| SOCS3 | R | ACD | 469931 | N/A | None | 2 hr | Opal | Perkin | N/A | N/A |
| | | Bio | | | | 40° C. | 690 | Elmer | | |
| SPP1 | R | ACD | 420101-C2 | N/A | 1:50 | 2 hr | Opal | Perkin | N/A | N/A |
| | | Bio | | | | 40° C. | 620 | Elmer | | |
| HAVCR2 | R | ACD | 560681-C3 | N/A | 1:50 | 2 hr | Opal | Perkin | N/A | N/A |
| | | Bio | | | | 40° C. | 650 | Elmer | | |
| CD68 | R | ACD | 560591-C4 | N/A | | 2 hr | Opal | Perkin | N/A | N/A |
| | | Bio | | | | 40° C. | 540 | Elmer | | |
| CD4 | P | Invitrogen | MA5- | Mouse | 1:100 | 16 hr | AF | BTCam | ab150105 | 1:100 |
| | | | 12259 | IgG | | 4° C. | 488 | | | |
| CD3 | P | BTCam | ab1669 | Rabbit | 1:200 | 16 hr | AF | Southern | 4050-32 | 1:100 |
| | | | | IgG | | 4° C. | 555 | Biotech | | |

R = RNA; P = protein

Statistical Analysis: For differential expression analysis, the embedded SCANPY function was used to identify differentially expressed genes in each cluster compared to the union of the rest of the clusters which used Benjamini-Hochberg method to control the false discovery rate. For specific comparisons of differential gene expression between cell types, MAST was used to calculate fold change and significance, based on a model incorporating cellular detection rate (based on number of genes per cell), gender, and patient as covariates. For frequency proportions, weighted least squares was used to adjust for number of cells sequenced in each individual and Benjamini-Hochberg method was used to adjust p-values for multiple comparisons. To assess the correlations of the frequency of cell types, Spearman's rank correlation coefficient was used. Flow cytometry data was analyzed with FlowJo (FlowJo Software for Mac Version 10, 2019) for data analysis, and two-sample t-test was performed using GraphPad Prism version 8.3.0 to compare frequency of cell types between patients and cancer-free subjects. For in vitro SOCS3 induction experiments, combined experiments were combined due to the small n in each individual experiment, using the fold change in percentage of SOCS3 for each group compared to the T cells alone control to normalize across experiments. A Wilcoxon test of the median of fold change for individuals was used to control for different patient samples used.

Survival Analysis of TCGA Data: Raw gene expression counts were downloaded from cholangiocarcinoma, prostate cancer, and colon cancer datasets using The Cancer Genomics Cloud; additional clinical metadata was downloaded from cBioportal. Overall survival (OS) and disease-free survival (DFS) were defined as from the time of collection of tissues to the date of death or last follow-up and estimated by the Kaplan-Meier method. As a starting point, the top 20 differentially expressed genes in CD14_{CTX} were used, as determined by MAST, and then only genes found in both datasets were used. A normalized z score was used for each gene, which was calculated by following formula:

$$\frac{raw gene expression - mean expression}{standard deviation of expression}$$

And then the composite score was calculated as the linear combination of the coefficients estimated based on the multivariable Cox proportional hazards (CPH) model (which includes all the top 20 genes) multiplied by the corresponding gene expression values. When fitting the CPH model, panelized regression with LASSO (least absolute shrinkage and selection operator) method was applied to avoid overfitting. The OS between patients who had the higher composite score (above the median) versus those with the lower score by log rank test was compared.

### Example 2. Multiplexed CITE-Seq Identified Altered Circulating Immune Cell Composition in BTC Patients Compared to Cancer-Free Subjects

Multiplexed CITE-seq was used to profile peripheral blood mononuclear cells (PBMCs) obtained from biliary tract cancer (BTC) patients (n=9). The PBMCs were obtained prior to, one week after, and three weeks after anti-PD-1 treatment. Control PBMCs were obtained from gender and age-matched cancer-free subjects (n=8) (Table 4, FIG. 1A). Over 230,000 cells were analyzed to identify the canonical circulating myeloid and lymphoid cell types, including B cells, CD4⁺ and CD8⁺ T cells, NK cells, NK T cells, plasmacytoid and conventional dendritic cells (pDC, cDC), CD14⁺ and CD16⁺ monocytes, plasma cells, and a small immune progenitor cell population. The cells were visualized using Uniform Manifold Approximation and Projection (UMAP) (FIG. 1B; FIGS. 9A-9E; Table 1). It was observed that cancer-free subjects and BTC patients had differences in the composition of broadly defined circulating immune cells. Baseline frequencies of CD8⁺ and plasma cells in BTC patients were decreased compared to those of cancer-free subjects (FIG. 1C; Table 5). While no difference in frequency was seen in the monocyte compartment overall, the transcriptional state of the monocyte compartment was distinct between BTC patients and cancer-free subjects both pre- and post-treatment (FIG. 1D). To explore whether anti-PD-1 could potentially have a direct effect on myeloid cells, the expression of PD-1 and its ligands were examined. In addition to PD-L1 and PD-L2 transcript and protein expression, PD-1 surface protein expression was observed in myeloid cells (particularly in CD14⁺ monocytes) before immune checkpoint inhibitor (CPI) treatment. These results suggested that anti-PD-1 can also act on myeloid cells (FIG. 1E).

TABLE 4
Clinical characteristics of BTC patents and cancer-free subjects

| | | | | | Cancer | | | |
|---|---|---|---|---|---|---|---|---|
| | | | | | stage at | | | Clinical |
| ID | Age | Gender | Race | Ethnicity | diagnosis | Tumor type | Viral etiology | responder |
| BTC-1 | 54 | M | White | Non- | Stage | Intrahepatic | No | No |
| | | | | Hispanic | IVB | cholangio- | | |
| | | | | | | carcinoma | | |
| BTC-2 | 65 | M | Asian | Non- | Stage | Intrahepatic | HBV+ (core | Yes |
| | | | | Hispanic | IVB | cholangio- | antibody | |
| | | | | | | carcinoma | positive, | |
| | | | | | | | surface antigen | |
| | | | | | | | negative) | |
| BTC-3 | 58 | F | White | Non- | Stage | Intrahepatic | No | Yes |
| | | | | Hispanic | IVB | cholangio- | | |
| | | | | | | carcinoma | | |
| BTC-4 | 68 | F | White | Non- | Stage | Intrahepatic | No | No |
| | | | | Hispanic | IVB | cholangio- | | |
| | | | | | | carcinoma | | |
| BTC-5 | 62 | M | White | Non- | Stage | Intrahepatic | No | No |
| | | | | Hispanic | IVB | cholangio- | | |
| | | | | | | carcinoma | | |
| BTC-6 | 53 | F | Asian | Non- | Stage III | Extrahepatic | No | Yes |
| | | | | Hispanic | | cholangio- | | |
| | | | | | | carcinoma | | |
| BTC-7 | 66 | F | White | Non- | Stage | Intrahepatic | No | Yes |
| | | | | Hispanic | IVB | cholangio- | | |
| | | | | | | carcinoma | | |
| BTC-8 | 61 | F | White | Non- | Stage II | Intrahepatic | No | No |
| | | | | Hispanic | | cholangio- | | |
| | | | | | | carcinoma | | |
| BTC-9 | 73 | M | Asian | Non- | Stage | Extrahepatic | HBV+ (core | No |
| | | | | Hispanic | IVB | cholangio- | antibody | |
| | | | | | | carcinoma | positive, | |
| | | | | | | | surface antigen | |
| | | | | | | | negative) | |
| HC-1 | 68 | M | White | Non- | N/A | N/A | N/A | N/A |
| | | | | Hispanic | | | | |
| HC-2 | 71 | F | Asian | Non- | N/A | N/A | N/A | N/A |
| | | | | Hispanic | | | | |
| HC-3 | 77 | M | White | Non- | N/A | N/A | N/A | N/A |
| | | | | Hispanic | | | | |
| HC-4 | 46 | M | Asian | Non- | N/A | N/A | N/A | N/A |
| | | | | Hispanic | | | | |
| HC-5 | 50 | F | Asian | Non- | N/A | N/A | N/A | N/A |
| | | | | Hispanic | | | | |
| HC-6 | 50 | F | White | Non- | N/A | N/A | N/A | N/A |
| | | | | Hispanic | | | | |
| HC-7 | 57 | M | Asian | Non- | N/A | N/A | N/A | N/A |
| | | | | Hispanic | | | | |
| HC-8 | 50 | F | White | Non- | N/A | N/A | N/A | N/A |
| | | | | Hispanic | | | | |

TABLE 5
P-values and fold changes for immune cell frequency comparisons

| | Fold | | Adjusted | Fold | | Adjusted |
|---|---|---|---|---|---|---|
| | change | p-value | p-value | change | p-value | p-value |
| | Cancer-free subjects v. BTC patients | | | BTC responders v. non-responders | | |
| Cell type | pre-treatment | | | pre-treatment | | |
| B cell | −0.03011 | 0.151701 | 0.2085889 | −0.029232 | 0.488881 | 0.6722114 |
| CD4⁺ T cell | 0.016203 | 0.805537 | 0.881293 | −0.009724 | 0.946072 | 0.988761 |
| CD8⁺ T cell | −0.13046 | 0.004156 | 0.037686 | −0.000291 | 0.988761 | 0.988761 |
| NK | −0.02485 | 0.140799 | 0.2085889 | 0.035257 | 0.831595 | 0.988761 |
| NK/NKT | 0.047816 | 0.088737 | 0.1626845 | −0.049857 | 0.097411 | 0.2678802 |
| cDC | 0.006557 | 0.024243 | 0.0630146 | −0.055735 | 0.071977 | 0.2639157 |
| CD14⁺ | 0.113645 | 0.028643 | 0.0630146 | 0.105051 | 0.1651 | 0.36322 |
| monocyte | | | | | | |
| CD16⁺ | 0.001102 | 0.881293 | 0.881293 | 0.005143 | 0.488069 | 0.6722114 |
| monocyte | | | | | | |
| pDC | 0.003942 | 0.018931 | 0.0630146 | −0.004237 | 0.258185 | 0.4733392 |
| plasma cell | −0.000891 | 0.006852 | 0.037686 | −0.00084 | 0.040534 | 0.222937 |
| progenitor | −0.002954 | 0.303424 | 0.3708516 | 0.004464 | 0.003466 | 0.038126 |
| | BTC responders v. non-responders | | | BTC responders v. non-responders | | |
| Cell type | at 1 week | | | at 3 week | | |
| B cell | −0.065438 | 0.205547 | 0.7536723 | −0.082013 | 0.078001 | 0.2860037 |
| CD4⁺ T cell | −0.006977 | 0.926735 | 0.926735 | 0.233162 | 0.013425 | 0.147675 |
| CD8⁺ T cell | 0.007326 | 0.534267 | 0.926735 | 0.025849 | 0.039419 | 0.2168045 |
| NK | 0.066213 | 0.615903 | 0.926735 | −0.084377 | 0.355565 | 0.4763022 |
| NK/NKT | −0.00677 | 0.858393 | 0.926735 | −0.044454 | 0.157034 | 0.3454748 |
| cDC | −0.005245 | 0.910505 | 0.926735 | −0.051041 | 0.199473 | 0.3657005 |
| CD14⁺ | 0.007942 | 0.345301 | 0.926735 | 0.000884 | 0.258632 | 0.4064217 |
| monocyte | | | | | | |
| CD16⁺ | 0.007132 | 0.043644 | 0.480084 | 0.000672 | 0.846558 | 0.846558 |
| monocyte | | | | | | |
| pDC | −0.005103 | 0.094657 | 0.5206135 | −0.002906 | 0.128991 | 0.3454748 |
| plasma cell | 0.000143 | 0.818514 | 0.926735 | −0.000404 | 0.433002 | 0.4763022 |
| progenitor | 0.000777 | 0.837115 | 0.926735 | 0.004629 | 0.404162 | 0.4763022 |

### Example 3. Insensitive BTC Patients Possessed Distinct Circulating Immune Populations Prior to and Following Treatment

Circulating immune cells in the BTC patients from the foregoing Example 2 were examined to determine whether there were any differences when analyzed by their clinical outcome to the treatment. Circulating cell composition was dynamic for both patients whose tumors responded to anti-PD-1 (responder) or was insensitive (non-responder) (FIGS. 2A-2C). Dynamic changes in the composition within the monocyte compartment was observed (FIG. 2C). Broad cell frequencies were not significantly different between responders and non-responders prior to or with therapy (FIGS. 2D-2E; FIG. 9F; Table 5). These findings differ from those reported in melanoma patients, in which an increased frequency of circulating CD14⁺ monocytes was observed in patients whose tumor responded to immunotherapy prior to treatment. Krieg et al., Nat Med 24:144-153 (2018).

### Example 4. BTC Patients Harbored Distinct Populations of Circulating Myeloid Cells

The monocytes and dendritic cells from the foregoing Example 3 were re-clustered to focus further on the myeloid compartment. Seven sub-populations were identified, annotated using a combination of protein and RNA markers (FIGS. 3A-3B). These included conventional dendritic cells (CDC), plasmacytoid dendritic cells (pDC), CD16⁺ monocytes, and four subpopulations of CD14⁺ monocytes (CD14_{CTX}, CD14_{ISG}, CD14 _{IFL}, and CD14_{APC}). The four subpopulations of CD14⁺ monocytes were annotated by canonical immune-specific pathways using gene ontology enrichment analysis of up-regulated genes (FIG. 3C). Ashburner et al., Nature Genetics 25:25-29 (2000); Li et al., Nat Immunol 15:195-204 (2014).
CD14_{IFL} myeloid cells were enriched for pathways related to inflammation (e.g., pro-inflammatory cytokines and chemokines, NFκB signaling, and inflammasome function).
CD14_{APC} cells were enriched in monocyte differentiation and function and antigen processing and presentation.
CD14_{ISG} represented a smaller population of CD14^{low} monocytes with upregulated interferon response genes (ISG) and innate immune signaling.
CD14_{CTX} cells were enriched for chemotaxis molecules (e.g., chemokines, chemokine receptors, and pro-inflammatory cytokines).

CD14_{CTX} were also distinguished by their increased expression of CD63 and CD68 and lower expression of CD14 (FIGS. 3B-3E). These findings suggest that CD14_{APC}, CD14 _{IFL}, and CD14_{ISG} are canonical CD14⁻ monocytes while CD14_{CTX} can exist on the spectrum of monocytes-macrophages. Betjes et al., Immunobiology 182:79-87 (1991); Iqbal et al., Blood 124: e33-44 (2014). The distribution of CD14⁺ sub-populations varied between BTC patients and cancer-free subjects with quantitative differences were detected for several populations, despite there not being an apparent difference when comparing total CD14⁺ monocyte frequencies overall (FIGS. 3D-3F; Table 6) In comparison to cancer-free subjects, BTC patients prior to treatment had a decreased frequency of CD14_{IFL} and CD16⁺ monocytes and an increased frequency of CD14_{APC} (FIG. 3F). In addition, CD14_{CTX} and CD14_{IsG} were exclusively found in the circulation of BTC patients and not cancer-free subjects (FIG. 3F).

TABLE 6
P-values and fold changes for myeloid cell frequency comparisons

| | Fold | | Adjusted | Fold | | Adjusted |
|---|---|---|---|---|---|---|
| | change | p-value | p-value | change | p-value | p-value |
| | Cancer-free subjects v. BTC patients | | | BTC responders v. non-responders | | |
| Cell type | pre-treatment | | | pre-treatment | | |
| CD14_{CTX} | 0.106337 | 0.19844 | 0.277816 | −0.085725 | 0.592656 | 0.963311 |
| CD14_{ISG} | 0.050707 | 0.513194 | 0.5987263 | 0.115711 | 0.446594 | 0.963311 |
| CD14_{IFL} | −0.503573 | 0.000003 | 0.000021 | −0.057482 | 0.001078 | 0.007546 |
| CD14_{APC} | 0.430743 | 0.006111 | 0.0213885 | 0.04452 | 0.836008 | 0.963311 |
| CD16 | −0.062957 | 0.044416 | 0.077728 | −0.001934 | 0.963311 | 0.963311 |
| monocytes | | | | | | |
| CDC | −0.01844 | 0.038108 | 0.077728 | −0.001074 | 0.929414 | 0.963311 |
| pDC | −0.002817 | 0.852105 | 0.852105 | −0.014015 | 0.61744 | 0.963311 |
| | BTC responders v. non-responders | | | BTC responders v. non-responders | | |
| Cell type | at 1 week | | | at 3 week | | |
| CD14_{CTX} | −0.056282 | 0.675966 | 0.788627 | −0.493558 | 0.005812 | 0.01356133 |
| CD14_{ISG} | 0.000916 | 0.632967 | 0.788627 | −0.000874 | 0.289613 | 0.4054582 |
| CD14_{IFL} | 0.049178 | 0.113661 | 0.656264 | 0.027247 | 0.649247 | 0.649247 |
| CD14_{APC} | −0.032417 | 0.796287 | 0.796287 | 0.519527 | 0.001245 | 0.0043575 |
| CD16 | 0.03109 | 0.426038 | 0.788627 | 0.021317 | 0.457643 | 0.53391683 |
| monocytes | | | | | | |
| CDC | 0.033795 | 0.187504 | 0.656264 | −0.03142 | 0.016753 | 0.02931775 |
| pDC | −0.02628 | 0.459307 | 0.788627 | −0.042238 | 0.000128 | 0.000896 |

### Example 5. Myeloid Subpopulation Frequencies and Gene Signatures Differed by Clinical Outcome

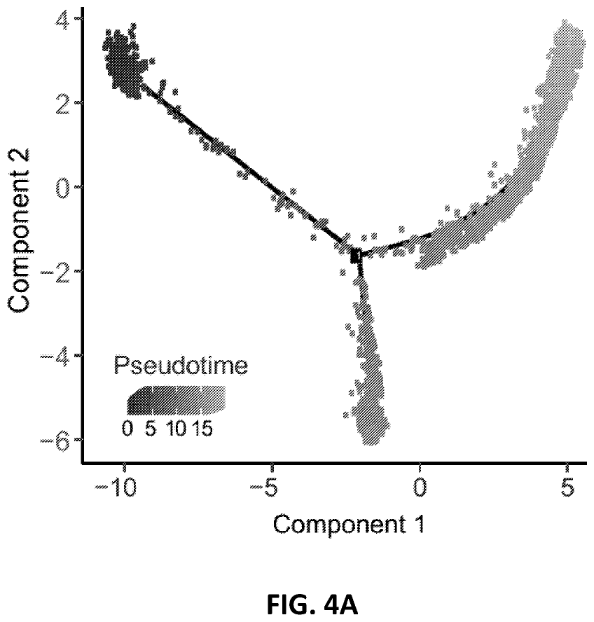

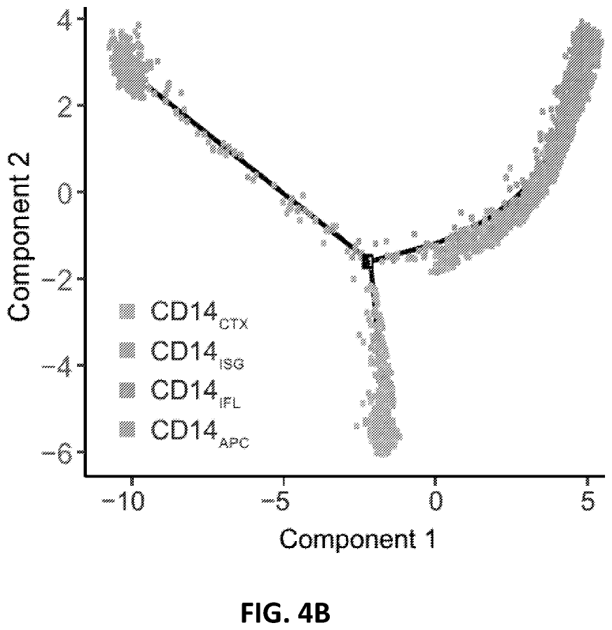

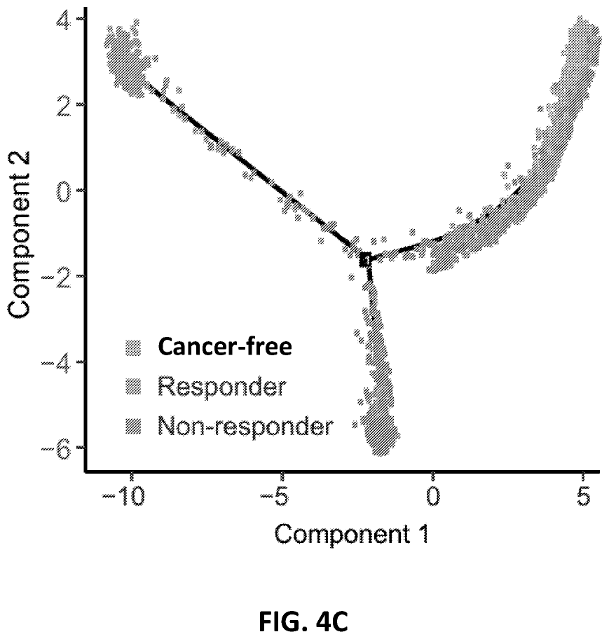

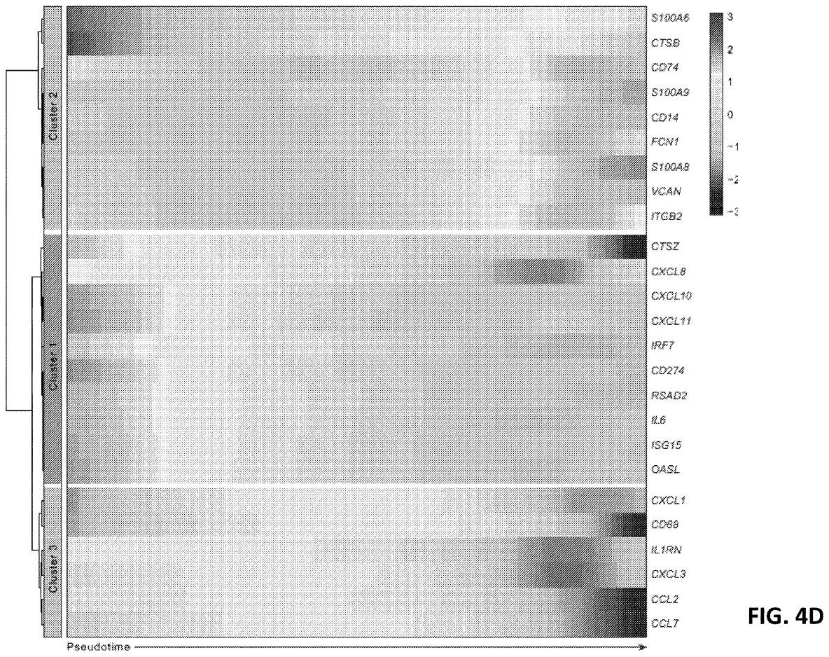

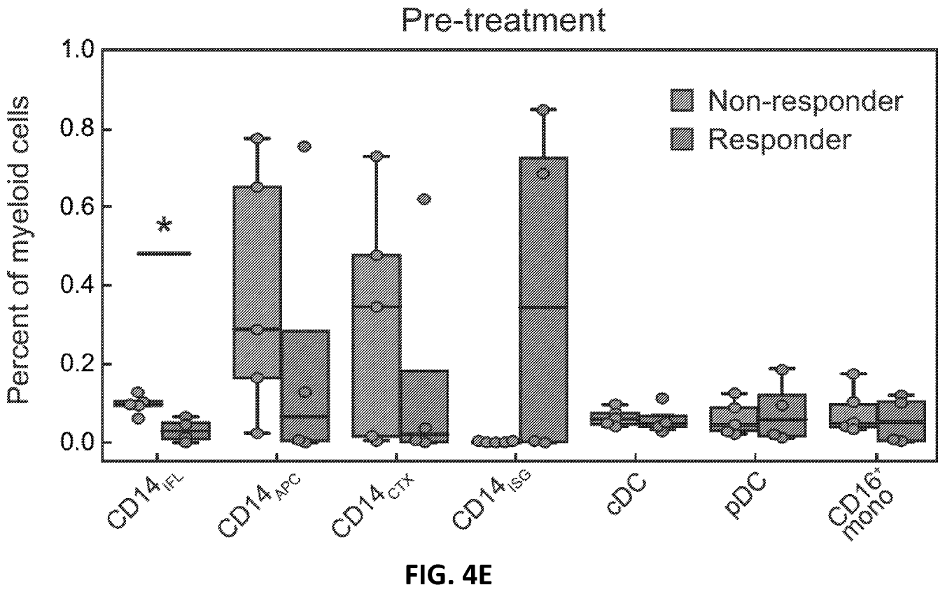

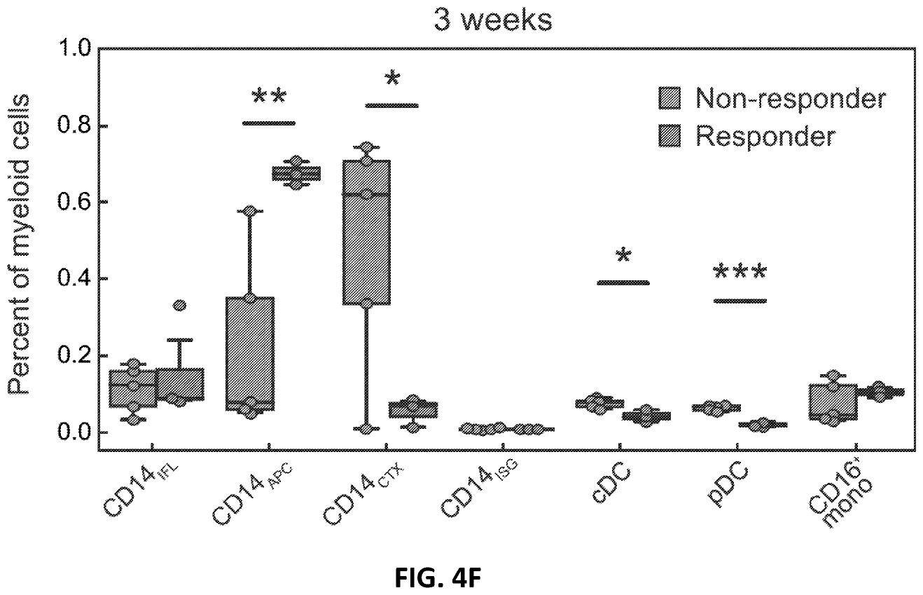

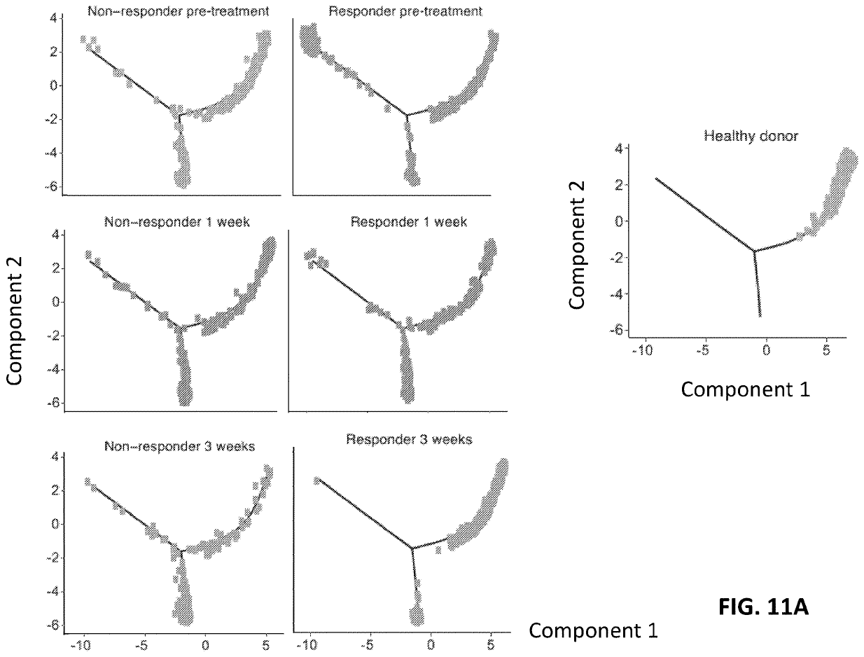

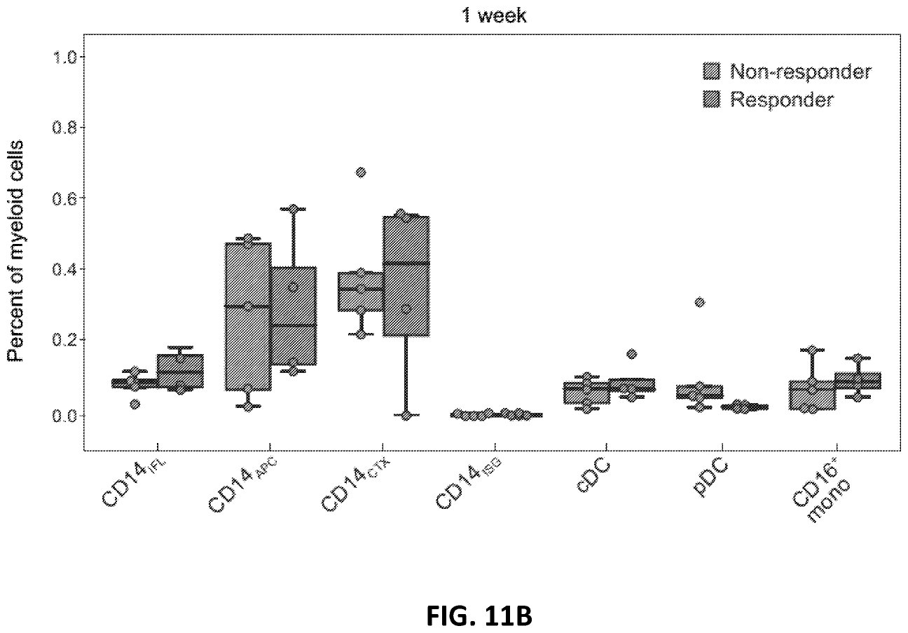

To examine whether the circulating monocyte subpopulations from the foregoing Example 4 may represent states of monocyte-macrophage differentiation, trajectory analysis from Trapnell et al., Nat Biotechnol 21:381-386 (2014) was used to order the four CD14⁺ monocyte sub-populations along pseudotime (FIG. 4A-4C). Genes differentially expressed along pseudotime overlapped with top differentially expressed genes in these populations and organized into several modules (FIG. 4D). Module 2 genes increased over pseudotime and included markers of monocytic lineage (CD14, VCAN, S100A8, S100A9, CD74), whereas modules 1 and 3 decreased over pseudotime and included ISGs (RSAD2, ISG15, IRF7), PD-L1 (CD274), and pro-inflammatory cytokines (CXCL8, CXCL10, CXCL11, IL6) (FIG. 4D). Furthermore, monocytes from BTC patients at baseline and 1 week were present across pseudotime in both responders and non-responders. However, by week 3, monocytes from responders were mainly found later in pseudotime alongside the CD14⁺ monocytes from cancer-free subjects (FIG. 4A; FIG. 11A). Differences across pseudotime between CD14⁺ sub-populations were noted. The composition of all myeloid populations was assessed by response category (FIGS. 4E-4F; FIG. 11B). The small population of CD14_{ISG} were derived from the circulation of two of the four responders at baseline (FIG. 4E). There were few other significant differences pre-treatment or at 1 week post-treatment between responders and non-responders (FIG. 4C; FIG. 11B). However, by three weeks post-anti-PD-1 treatment, responders had a markedly higher frequency of CD14_{APC}, whereas non-responders had an increased frequency of CD14_{CTX}, pDC, and cDC (FIG. 4F).

### Example 6. CD14_{CTX} Expressed a Program of Immunosuppressive Chemokines and Cytokines

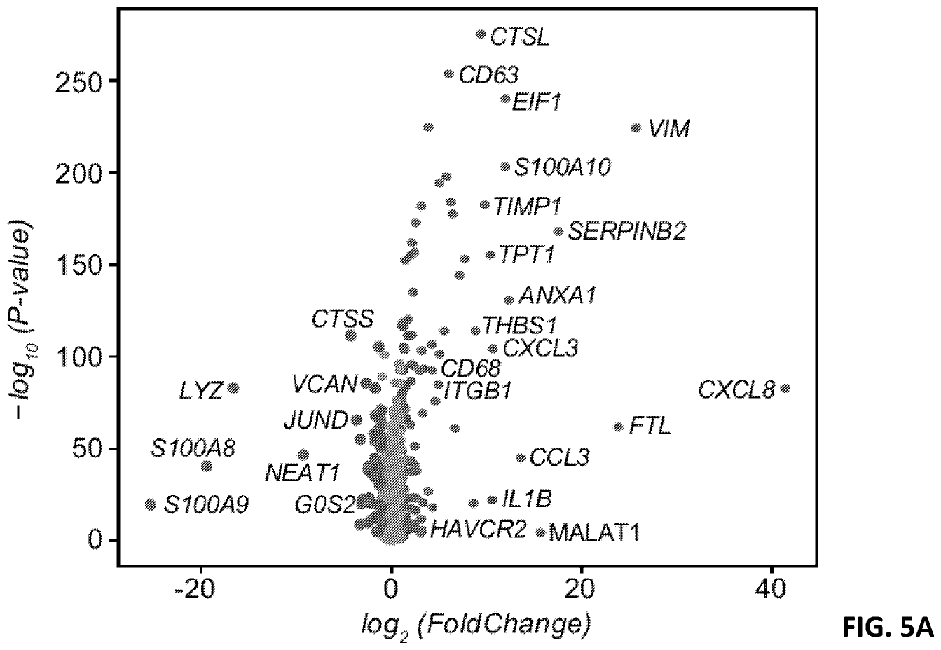

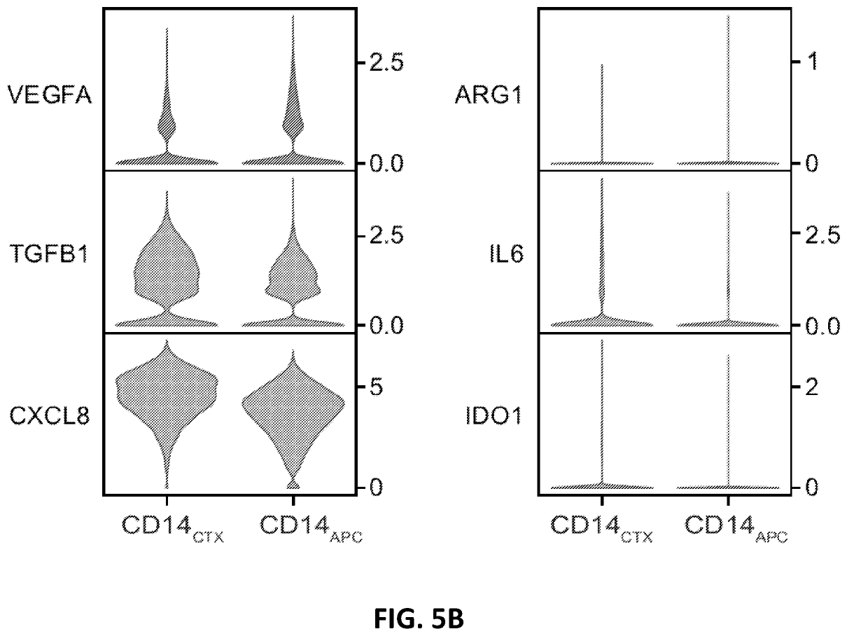

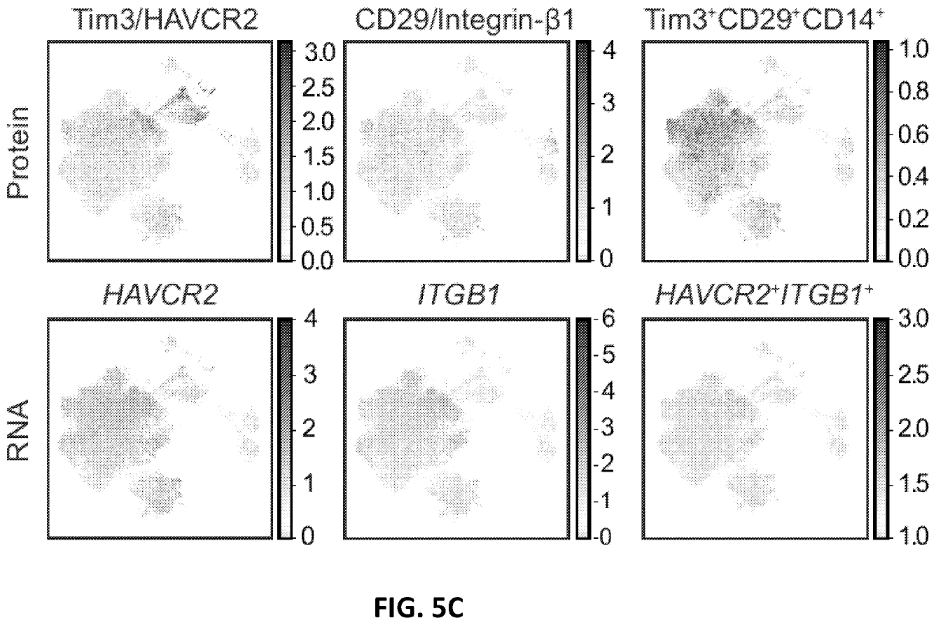

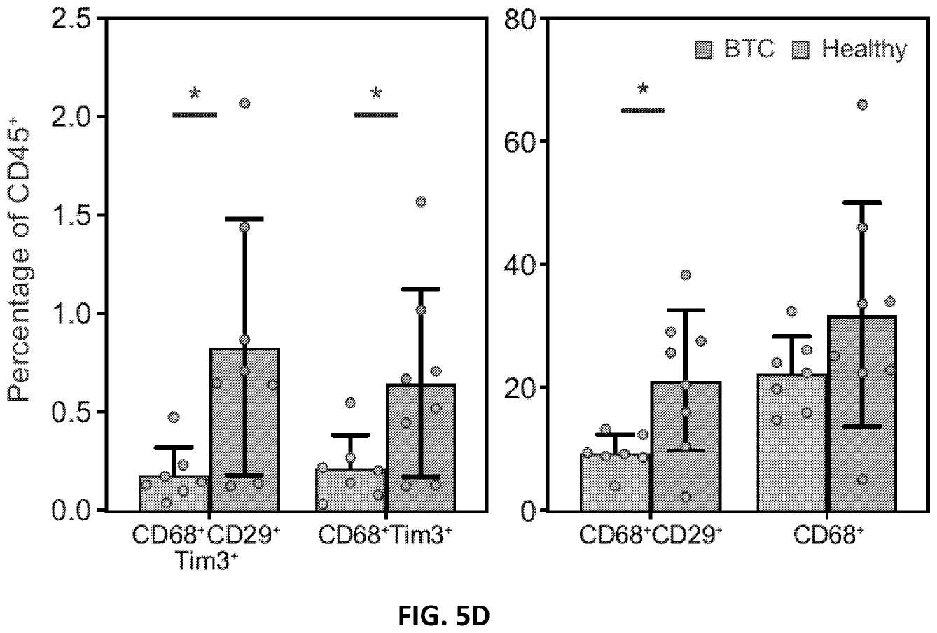

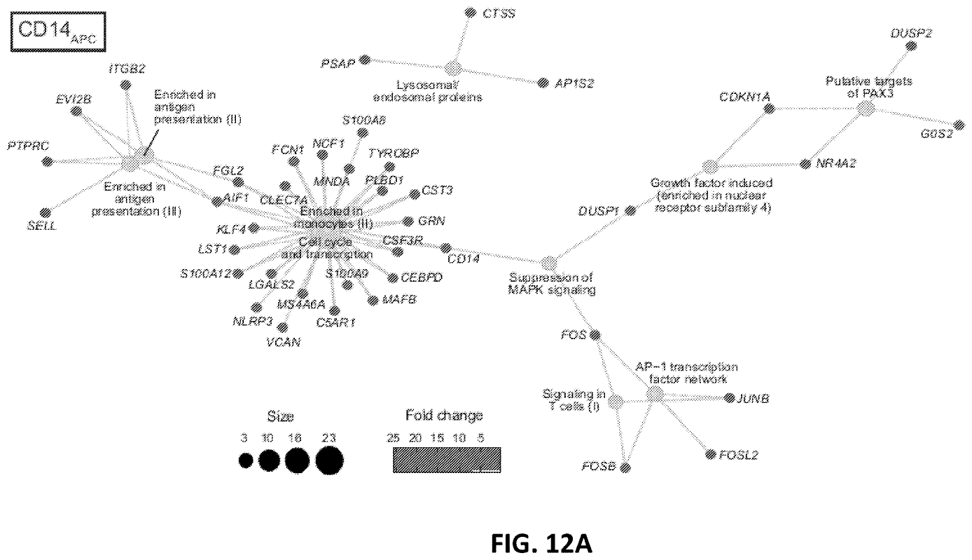

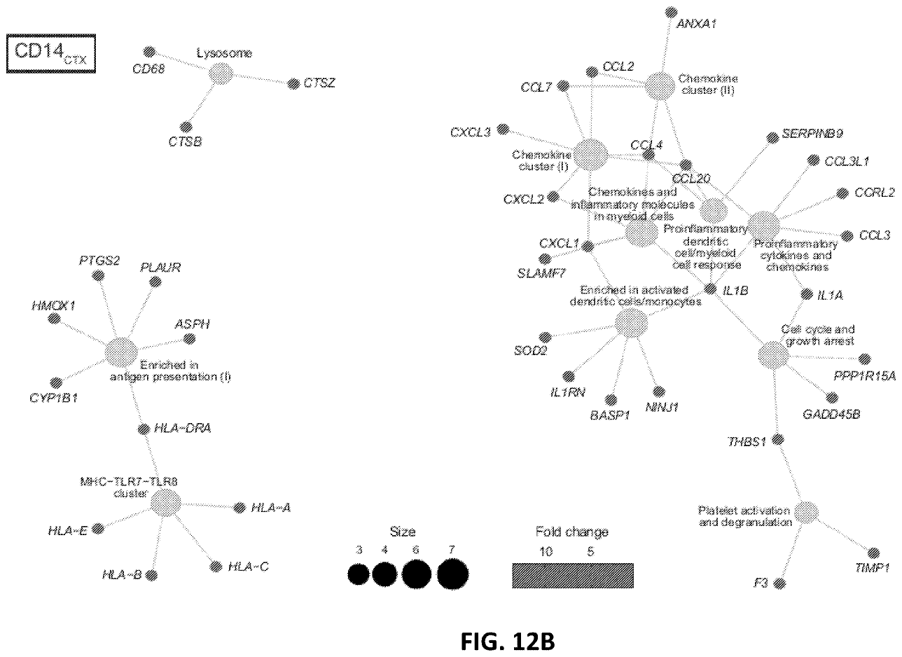

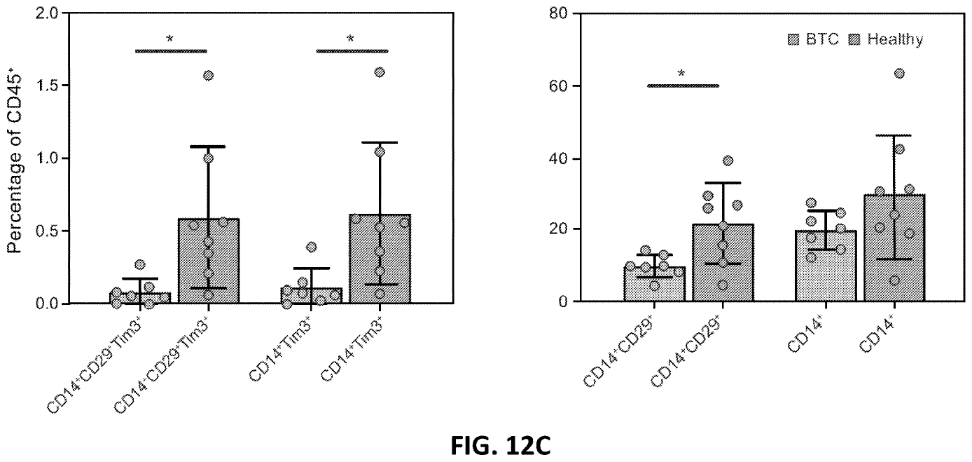

CD14_{CTX} to CD 14_{APC} cells from the foregoing Example 5 were compared using MAST. See Finak et al., Genome Biol 16:278 (2015) for MAST. CD14_{CTX} had increased expression of several tumor-associated macrophage (TAM) and/or myeloid-derived suppressor cell (MDSC)-related cytokines, including IL6, TGFBI, and CXCL8 (FIGS. 5A-5B). However, CD14_{CTX} lacked expression of other MDSC-associated genes including ARGI, VEGFA, and IDO1 (FIG. 5B). Although antigen processing and presentation pathways were enriched in both monocyte sub-population CD14_{CTX} and CD14_{APC}, the individual genes and pathways differed (FIGS. 12A-12B). CD14_{CTX} expressed COX2 (PTSG2) and HLA molecules (FIG. 12B), while CD 14_{APC} expressed genes related to monocyte surface phenotype (S100A8, S100A9, CD14, FCN1) and function (i.e., the inflammasome-related gene, NLRP3) (FIG. 5A; FIG. 12A). CD14_{CTX} also expressed a distinct set of chemokines involved in the recruitment of CCR2″ inflammatory monocytes, a population associated with poor outcomes in cancer patients (CCL2, CCL7), recruitment of neutrophils (CXCLI, CXCL2, CXCL3), and associated with T cell exhaustion (CCL20), pro-inflammatory cytokines (ILIA, ILIB), as well as molecules associated with cell migration and extracellular matrix digestion (TIMP1, CTSB, CTSZ) (FIG. 5A; FIG. 12B).

For further in vitro functional characterization, the surface protein abundance data from CITE-seq were used to identify markers that can distinguish of CD14_{CTX} from other monocyte subpopulations. First, COMET was used to identify two highly expressed surface markers in CD14_{CTX}: (1) Tim3 (HAVCR2), an immune checkpoint on T cells that is also expressed by dendritic cells and M2 macrophages; and (2) CD29 (ITGB1), an integrin that can mediate chemotaxis and is upregulated in macrophages compared to other myeloid cells. See Delaney et al., Mol Syst Biol 15: e9005 (2019) for COMET. High expression of Tim3 and CD29 by CD14_{CTX} at the RNA and protein levels were confirmed with UMAP. High expression of Tim3 and CD29 combination specifically distinguished CD14_{CTX} from other subpopulations (FIG. 5C). The BTC patients had an increased frequency of CD29 Tim3⁺CD68⁺ cells as well as Tim3⁺CD68⁺ and CD29⁺CD68⁺ cells compared to cancer-free subjects, with similar findings shown for CD14 gated cells (FIG. 5D; FIG. 12C). Enrichment of CD29 Tim3⁺ monocytes was specific to BTC patients, while the frequency of total CD14⁺ or CD68⁺ myeloid cells did not differ significantly between cancer-free subjects and BTC patients (FIG. 5D; FIG. 12C).

### Example 7. CD14_{CTX} Gene Signature Correlated with SPP1⁺ TAM in the Tumor Microenvironment and was Associated with Poor Prognosis in Other CPI-Insensitive Tumors

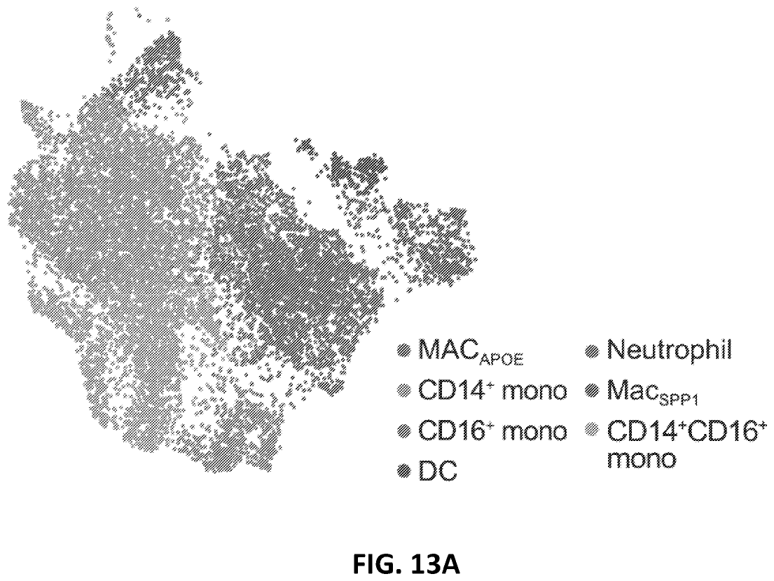

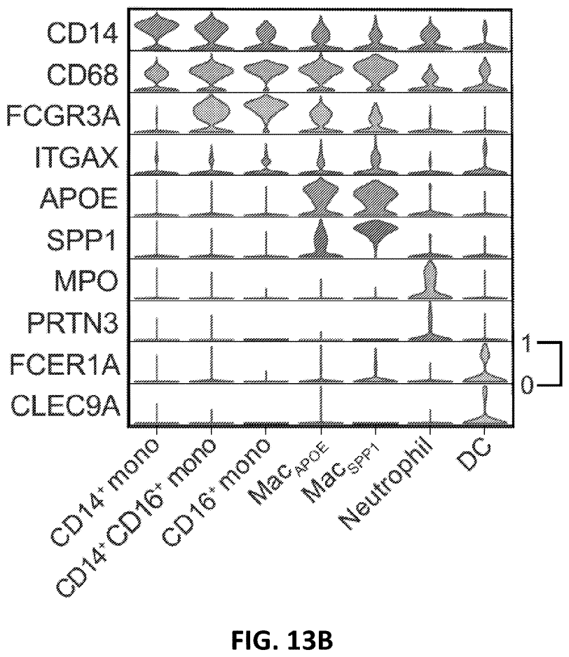

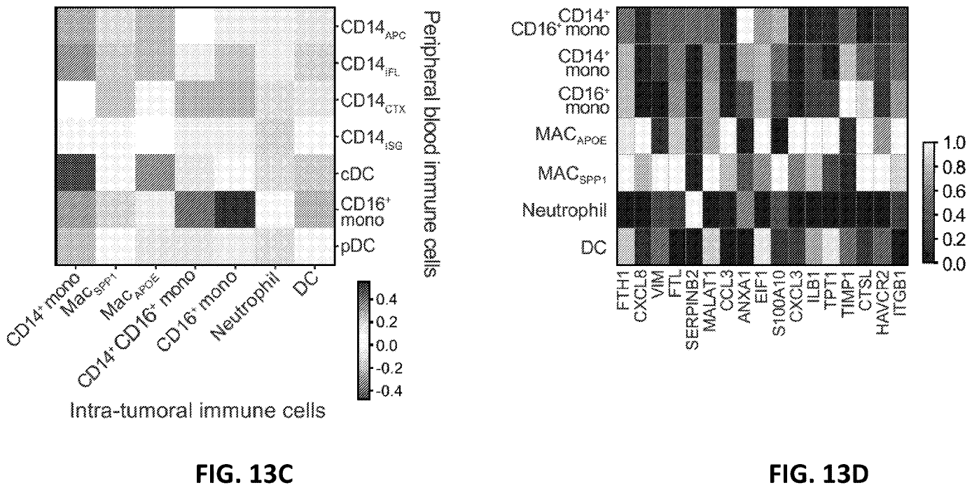

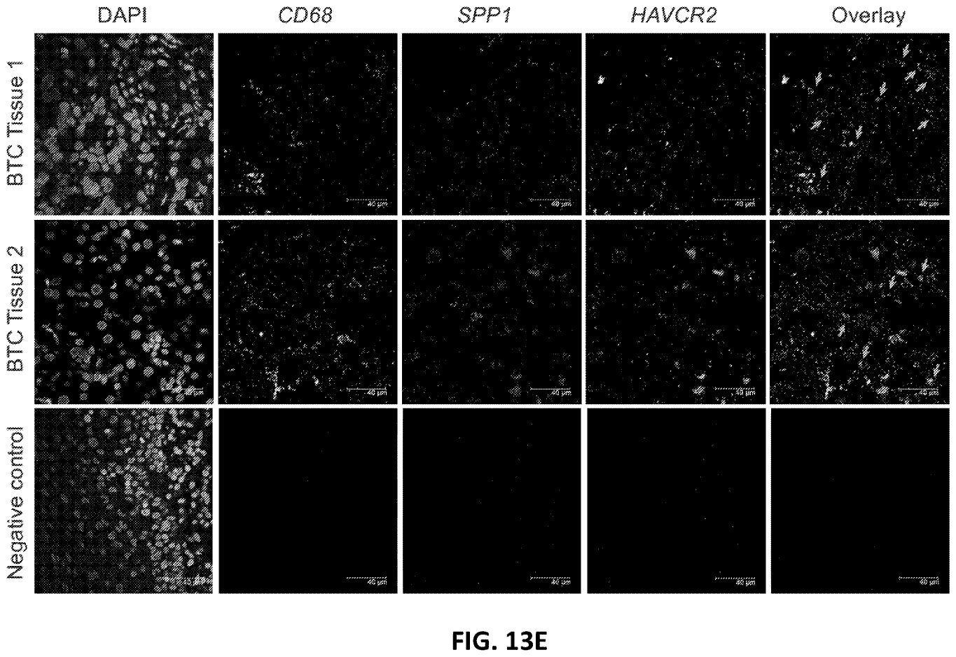

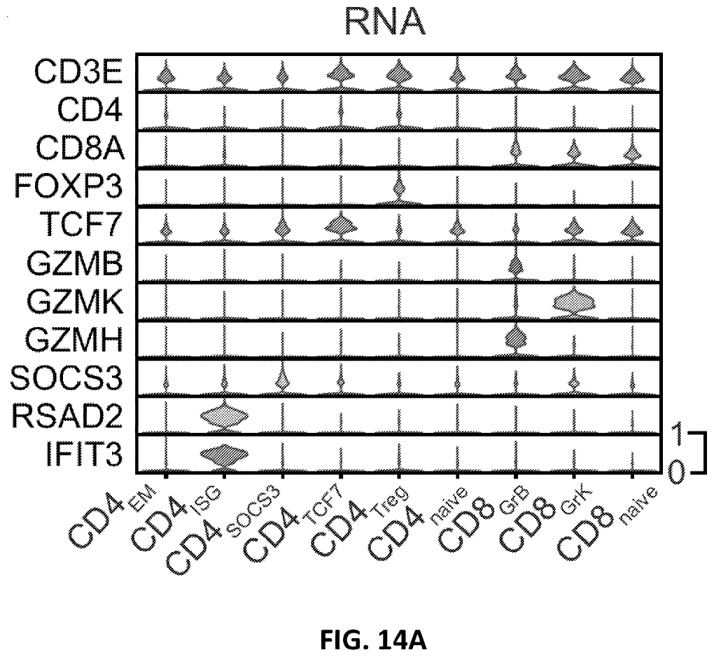

As shown in the foregoing Example 6, CD14_{CTX} expressed chemokine receptors that might facilitate migration into the tissues. Thus, the relationship between circulating and intra-tumoral myeloid states in BTC was examined next. scRNA-seq was performed on primary cholangiocarcinoma tumors (n=4) obtained from standard of care resections (Table 6) and a total of 10,913 myeloid cells were recovered. Tissue-associated myeloid cells were consisted of dendritic cells (DC), neutrophils (Neut), two populations of macrophages characterized by either high APOE expression (MaC_{APOE}) or high SPP1 expression (Mac_{SPP1}), CD14⁺ monocytes (CD14⁺ mono), CD16⁺ monocytes (CD16⁺ mono), and intermediate CD14⁺CD16⁺ monocytes (CD14⁺CD16⁺ mono) (FIGS. 13A-13B). Among the tissue-associated myeloid populations, the expression profile of CD14_{CTX} were most correlated with Mac_{SPP1}, exemplified by the shared expression of differentially expressed CD14_{CTX} genes including HAVCR2 and ITGB1 (FIGS. 13C-13D). Notably, two genes that differed in expression between MAC_{SPP1} and CD14crx are related to chemotaxis and extravasation (SERPINB2, TIMP 1), suggesting a transition in macrophages that had already migrated to the tumor microenvironment. Combination of in situ hybridization and immunofluorescence detected SPP1⁺HAVCR2⁺CD68⁺ myeloid cells within biliary tumor tissue from on-treatment biopsies, further suggesting the existence of a TAM population in BTC analogous to CD14_{CTX} (FIG. 13E).

TABLE 6
Clinical characteristics of BTC resection samples

| Sample | | | Age | | |
|---|---|---|---|---|---|
| number | Viral status | Prior treatment | (years) | Gender | Tumor classification |
| Tumor-1 | non-viral | None | 64 | Male | Extrahepatic |
| | | | | | cholangiocarcinoma |
| Tumor-2 | HBV cAb positive, | None | 43 | Female | Intrahepatic |
| | HBV sAg negative, | | | | cholangiocarcinoma |
| | HBV sAb positive | | | | |
| Tumor-3 | non-viral | 4 cycles | 60 | Male | Intrahepatic |
| | | gemcitabine/cisplatin, | | | cholangiocarcinoma |
| | | portal vein | | | |
| | | embolization | | | |
| Tumor-4 | non-viral | None | 37 | Male | Intrahepatic |
| | | | | | cholangiocarcinoma |

HBV = hepatitis B virus;
HCV = hepatitis C virus;
cAb = core antibody;
sAg = surface antigen;
sAb = surface antibody

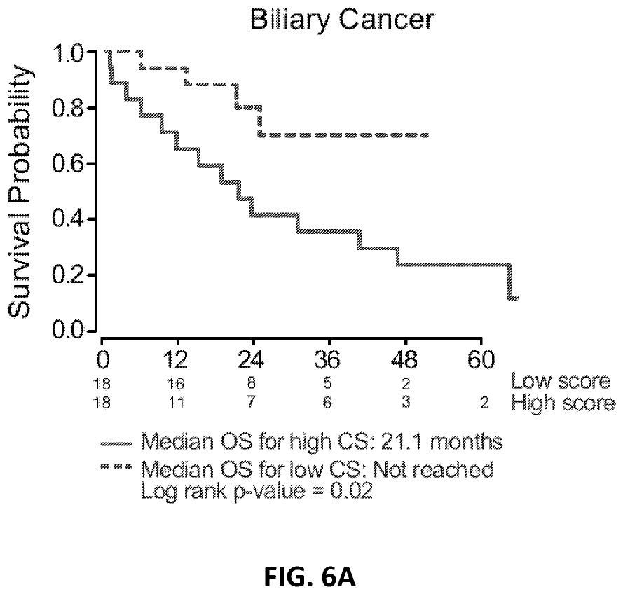

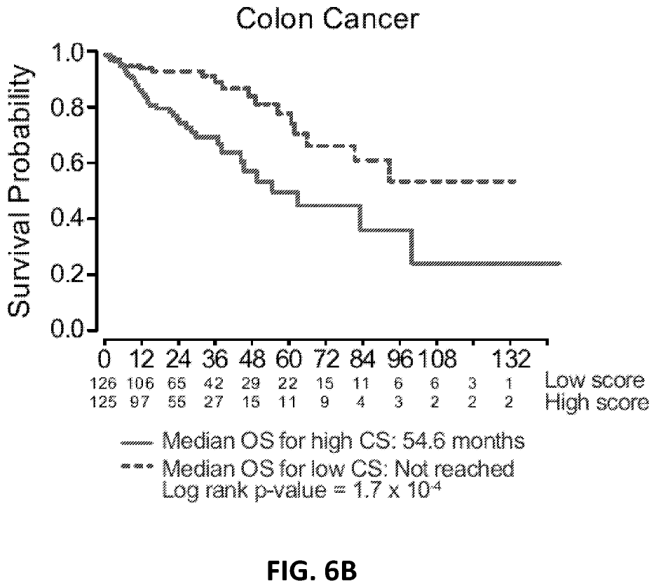

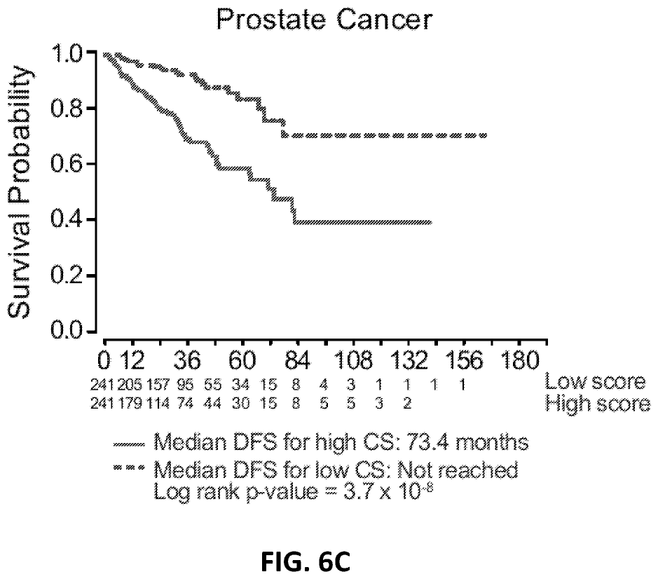

To test whether or not the CD14_{CTX} gene signature was prognostically relevant, the CD14_{CTX} gene signature was applied to the TCGA cholangiocarcinoma dataset (median overall survival=40.13 months, n=36). High expression of the CD14_{CTX} gene signature was indeed associated with a significantly worse overall survival (median survival=21.1 months v. not reached, p-value=0.02) (FIG. 6A). The CD14crx gene signature was applied to two other prototypical CPI insensitive cancers: colorectal and prostate cancers. The CD14_{CTX} gene signature was correlated with worse prognosis in both of these diseases as well. In colon cancer, a high CD14_{CTX} expression score correlated with overall survival of 54.6 months versus not reached for patients with a lower score (p=1.7×10⁻⁴, n=251) (FIG. 6B). In prostate cancer, a higher CD14_{CTX} gene signature expression score correlated with lower disease-free survival (DFS) (73.4 months v. not reached, p=3.7×10⁻⁸, n=482) (FIG. 6C).

### Example 8. CD14_{CTX} Frequency Correlated with CD4_{SOCS3} Cell Frequency

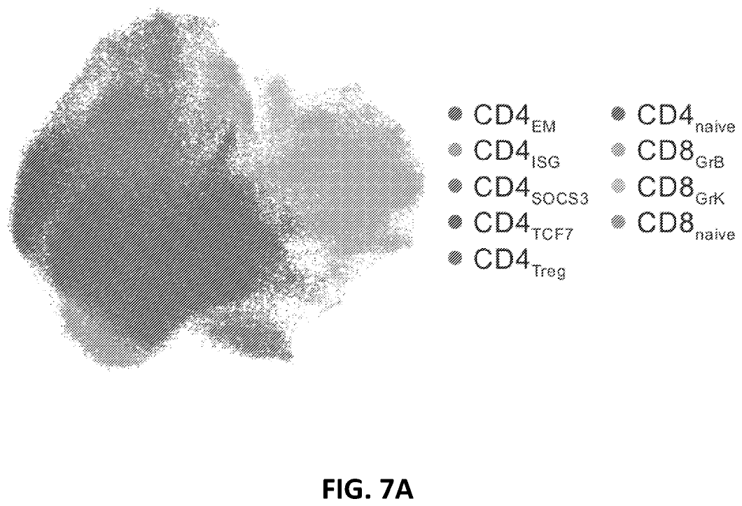

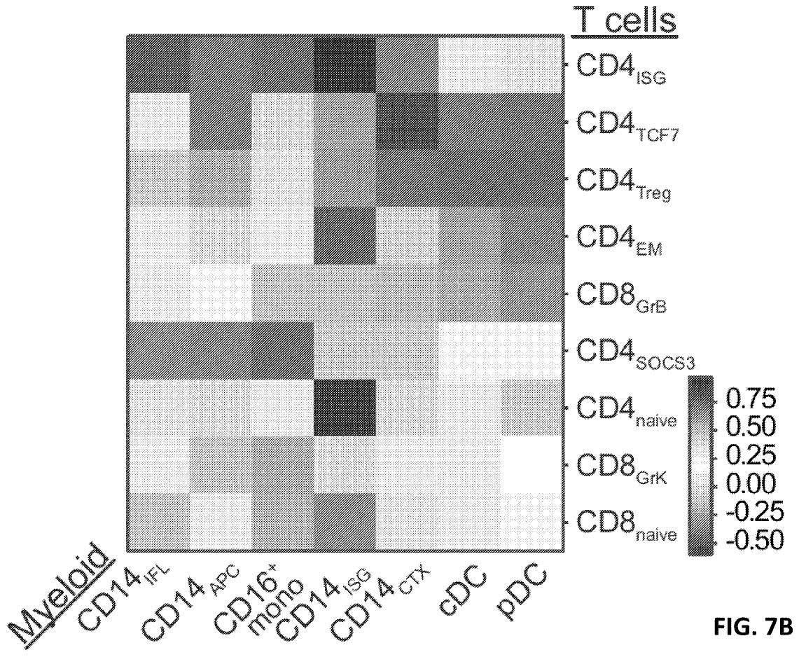

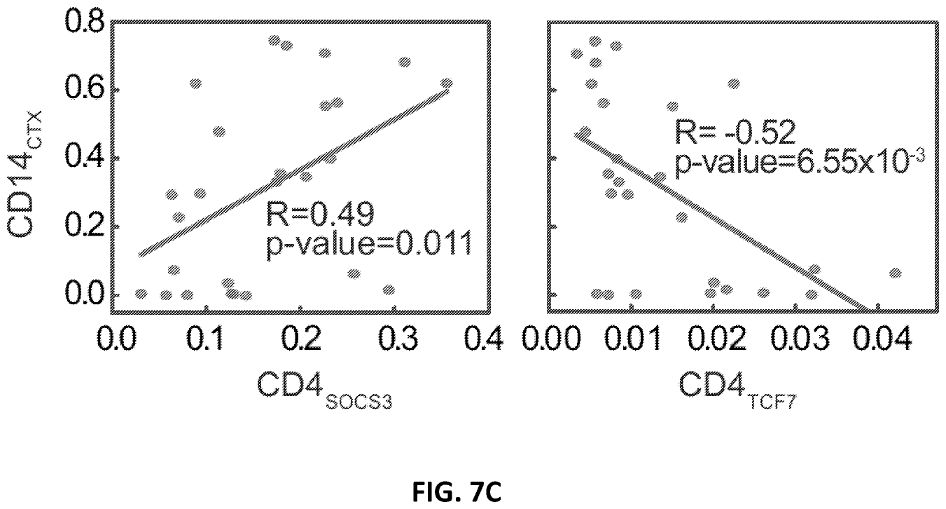

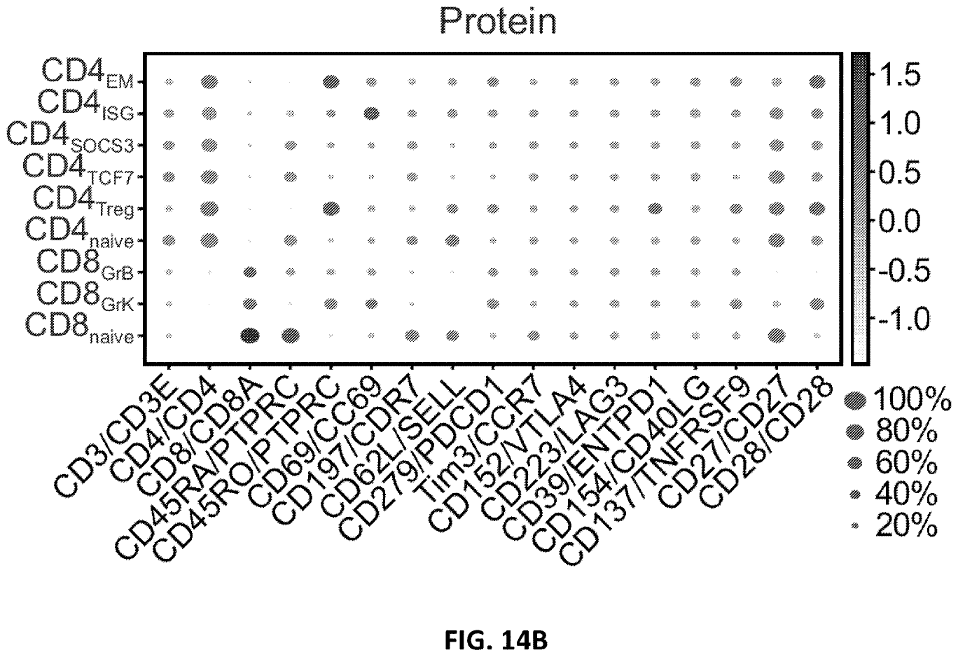

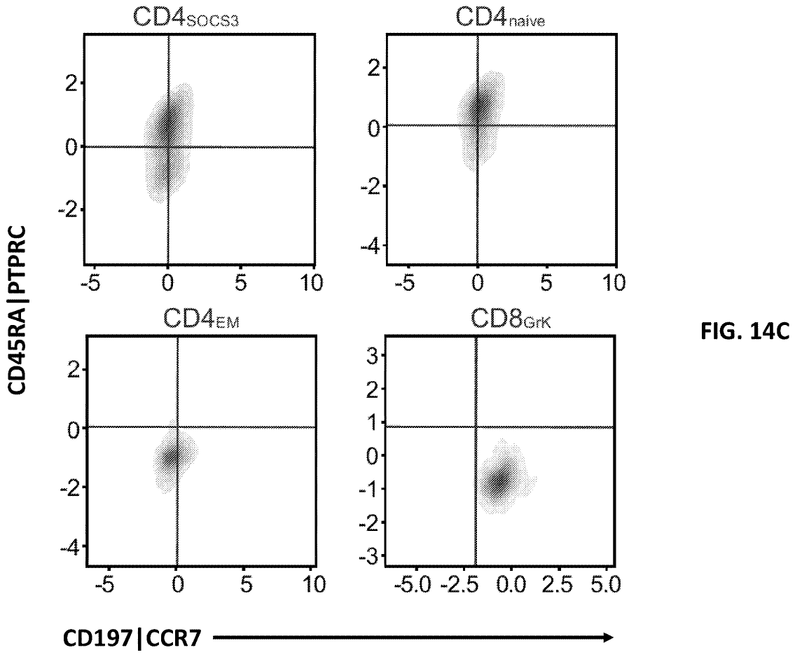

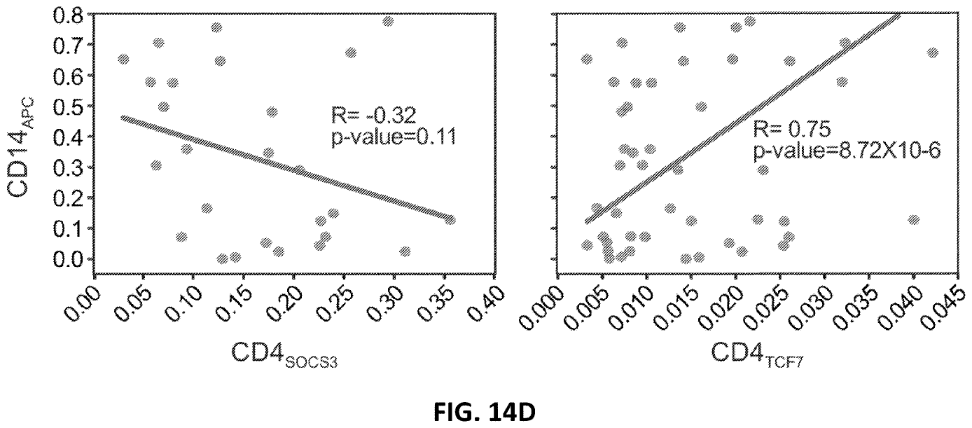

The CD4⁺ and CD8″ T cells were re-clustered to define T cell sub-populations present in cancer-free subjects and BTC patients (FIG. 7A). Nine unique T cell clusters were identified using both transcript and protein markers: six CD4⁺ T cell clusters including naïve and effector memory (CD4ₙₐᵢᵥₑ, CD4EM), FOXP3⁺ regulatory (CD4_{Treg}), and cells characterized by high expression of either TCF7, SOCS3, or ISG (CD4_{TCF7}, CD4_{SOCS3}, CD4_{ISG}); and three clusters of CD8⁺ T cells including naïve (CD8ₙₐᵢᵥₑ) and effectors expressing either predominantly GZMB/GZMH or GZMK (CD8_{GrB}, CD8_{GrK}) (FIGS. 14A-14C). To determine whether myeloid-T cell interactions were involved in CPI insensitivity, frequency association between myeloid cells and T cells sub-populations was examined (FIG. 7B). Surprisingly, in BTC patients, the frequency of CD14_{CTX} was positively correlated with the frequency of CD4_{SOCS3} (R=0.49, p-value=0.011) and negatively correlated with CD4TCF7 frequency (R=−0.52, p-value=6.55×10⁻³) (FIG. 7C), whereas the frequency of CD14_{APC} was positively correlated with the frequency of CD4_{TCF7} (R=0.75, p-value=8.72×10⁻⁶) and not correlated with the frequency of CD4_{SOCS3} (R=−0.32, p-value=0.11) (FIG. 14D). The positive correlation of CD4_{TCF7} with CD14_{APC} and negative correlation with CD14_{CTX} in BTC patients was unexpected because TCF7 expression within CD4⁺ T cells is associated with the capability to self-renew. Alternately, SOCS3 is a negative regulator of cytokine signaling and has been associated with T cell dysfunction.

### Example 9, CD14_{CTX} Suppressed CD4⁺ T Cells

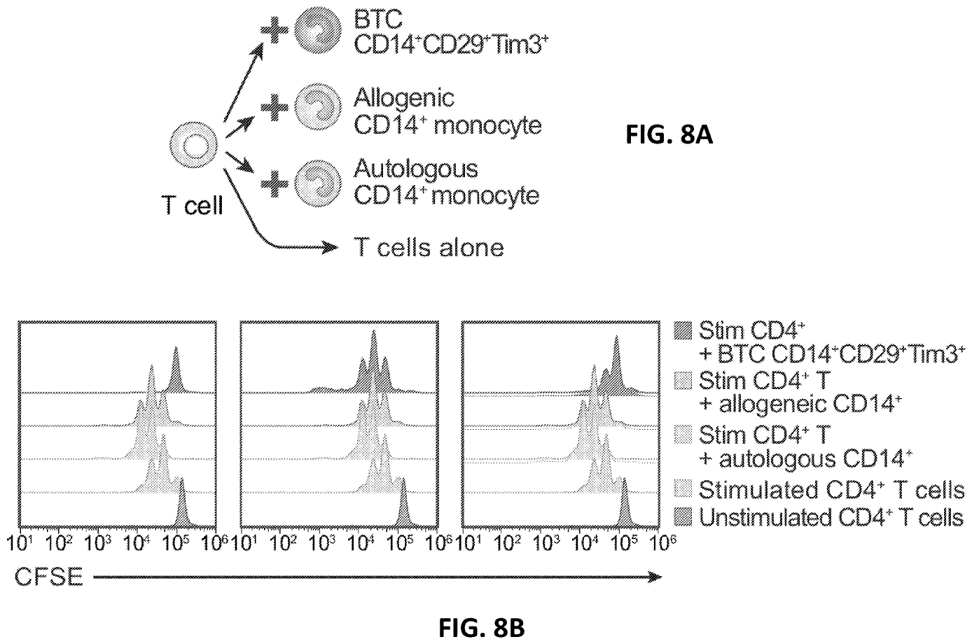

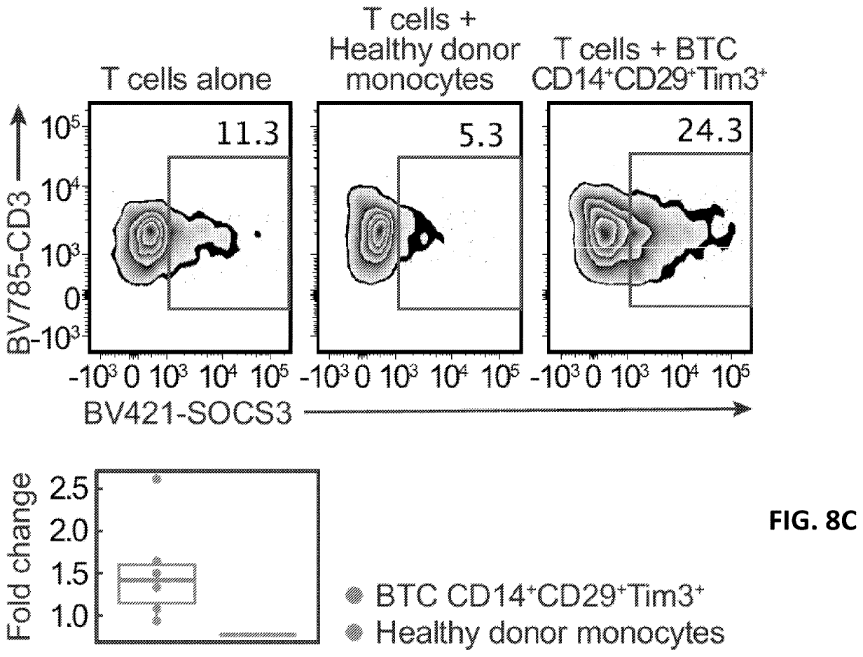

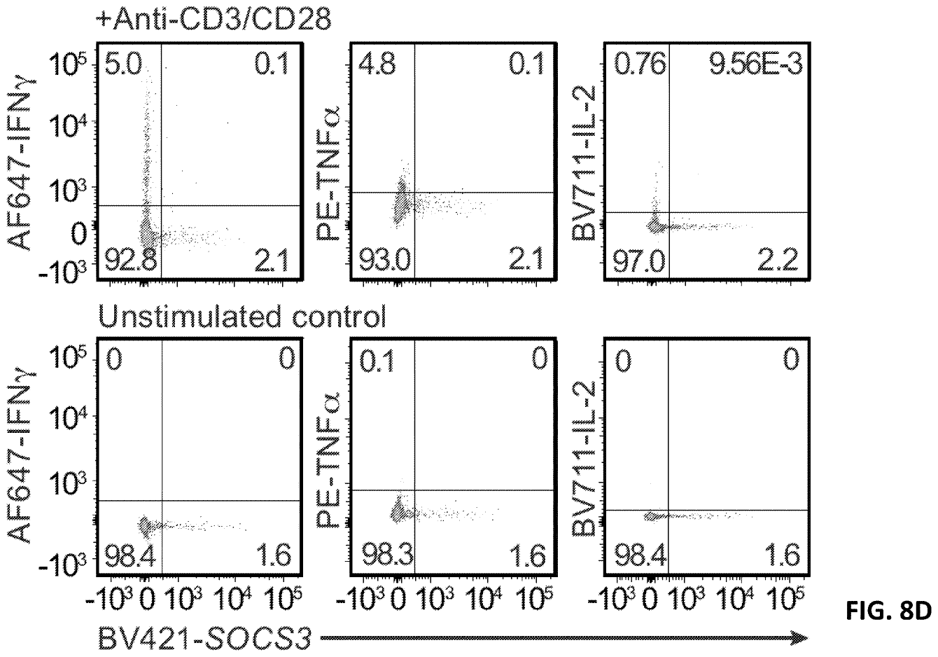

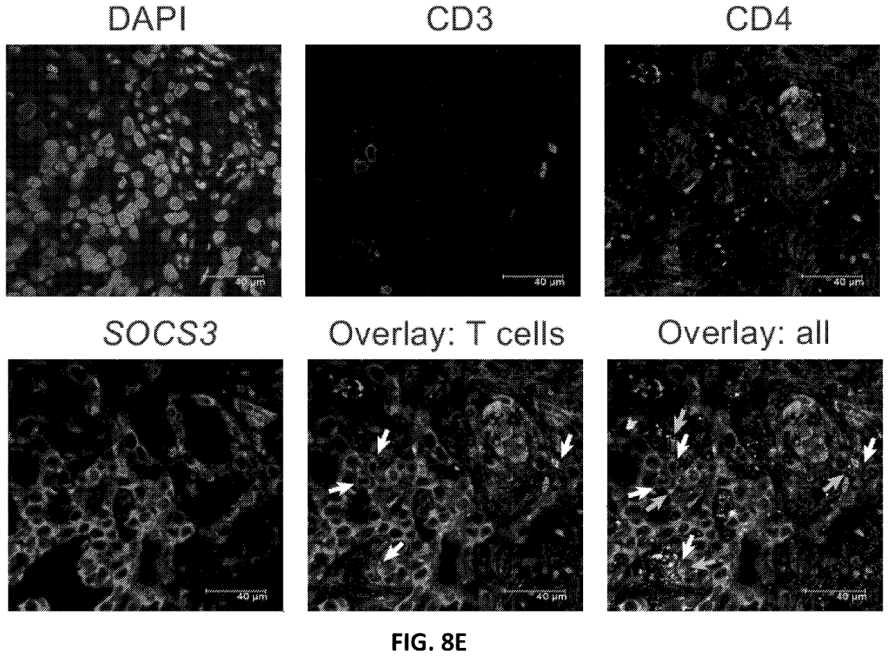

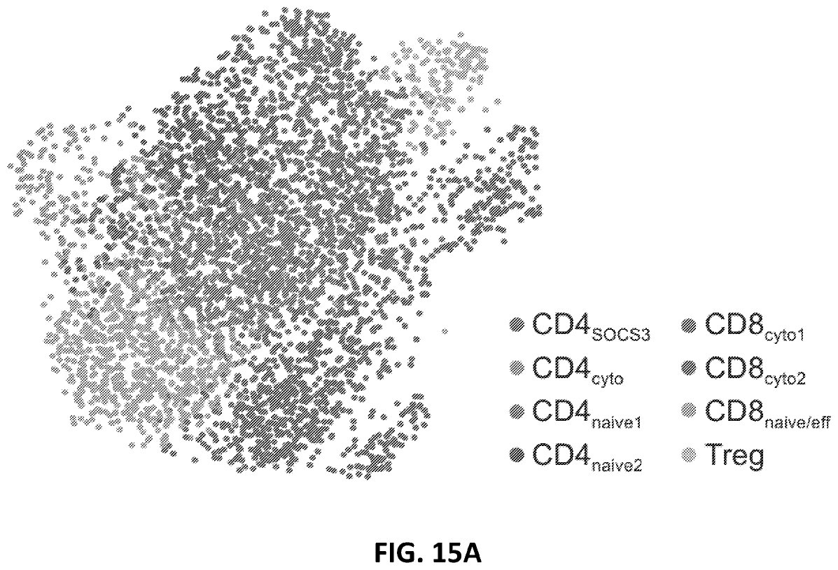

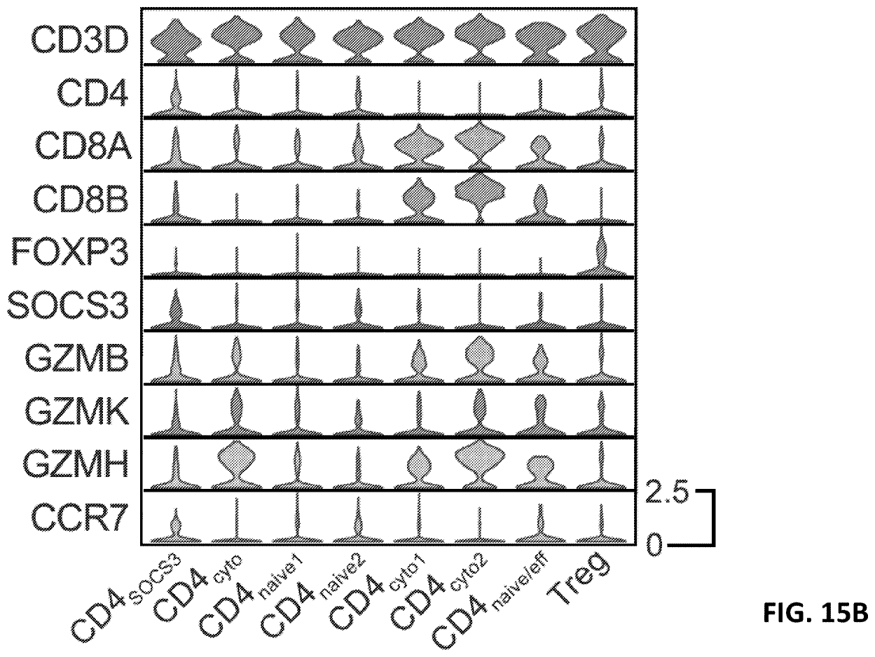

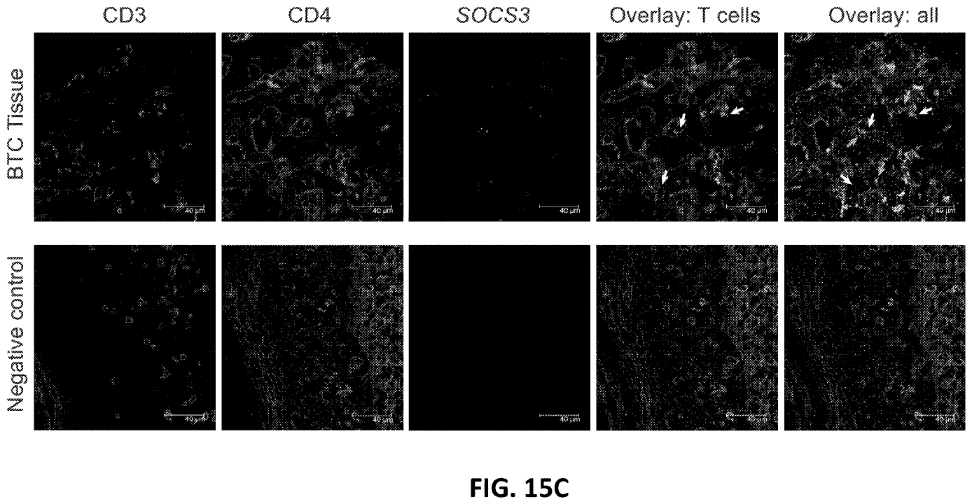

Next, the capacity of CD 14_{CTX} monocytes to alter the proliferation and function of CD4⁺ T cells were investigated in vitro. Using the markers identified from CITE-seq analysis and validated by flow cytometry (Tim3, CD29, CD14), CD14_{CTX} cells from BTC patients were isolated with fluorescence-activated cell sorting (FACS). The cells were co-cultured with cancer-free subject CD4⁺ T cells (FIG. 8A; FIGS. 10A-10B). Compared to autologous or allogeneic cancer-free subject CD14⁺ monocytes, CD14_{CTX} cells isolated from BTC patients' circulation suppressed the proliferation of CD4⁺ T cells (FIG. 8B). Further, CD14_{CTX} induced SOCS3 expression in sorted naïve CD4⁺ T cells compared to cancer-free subject monocytes (median fold change in each condition compared to cancer-free subject T cells alone for 3 experiments, p=0.031), consistent with the association between the frequency of CD14_{CTX} with CD4_{SOCS3} (FIG. 8C). SOCS3 expression is associated with “immune paralysis” in CD4⁺ T cells in the setting of cytokine exposure. Thus, the functional capacity of SOCS3⁺CD4⁺ T cells (CD4_{SOCS3} cells) induced by BTC-derived CD14crx monocytes was assessed. While CD4_{SOCS3} cells from biliary track cancer patients retained the ability to produce IFNγ, TNFα, and IL2, CD4_{SOCS3} cells failed to produce these cytokines in response to stimulation (FIG. 8D). Lastly, whether CD14_{CTX} and CD4_{SOCS3} interacted within the tumor microenvironment was examined because a population of CD4_{SOCS3} cells in biliary tumors was identified by scRNAseq (FIGS. 15A-15B). Foregoing results were corroborated in in situ hybridization, confirming not only the presence of CD3⁺CD4 SOCS3⁺ T cells in the human BTC tissue sections, but also their co-localization with HAVCR2 SPP1⁺CD68⁺ cells in the tumor microenvironment (FIG. 7E; FIG. 15C).

While the disclosure has been particularly shown and described with reference to specific embodiments (some of which are preferred embodiments), it should be understood by those having skill in the art that various changes in form and detail can be made therein without departing from the spirit and scope of the present disclosure as disclosed herein.

We claim:

1. A method of selecting a subject amendable to treatment with one or more immune checkpoint inhibitors from one or more subjects having or suspected of having cancer, the method comprising:
(a) isolating a test population of peripheral blood mononuclear cells (PBMCs) from the subject and one or more reference populations of PBMCs from each subject of the one or more subjects, wherein each reference population is isolated independently from each subject of the one or more subjects;
(b) quantifying frequency of CD14_{CTX} cells in the test population;
(c) quantifying frequency of CD14_{CTX} cells in the each reference population independently, and averaging the frequency CD14_{CTX} cells in the one or more reference populations;
(d) comparing the frequency of CD14_{CTX} cells in the test population to the average frequency of CD14_{CTX} in the one or more reference population; and
(e) selecting the subject as the subject amendable to the treatment if the frequency of CD14_{CTX} cells in the test population is lower than the average frequency of CD14_{CTX} cells in the one or more reference populations.

2. A method of treating cancer in a subject having or suspected of having the cancer with one or more immune checkpoint inhibitors, the method comprising:
(a) isolating a test population of PBMCs from the subject and one or more reference populations of PBMCs from one or more subjects having or suspected of having the cancer, wherein each reference population is isolated independently from each subject of the one or more subjects;
(b) quantifying frequency of CD14_{CTX} cells in the test population;
(c) quantifying frequency of CD14Crx cells in the each reference population independently, and averaging the frequency of CD14_{CTX} cells in the one or more reference populations;
(d) comparing the frequency of CD14_{CTX} cells in the test population to the average frequency of CD14_{CTX} in the one or more reference populations; and
(e) administering a therapeutically effective amount of the one or more immune checkpoint inhibitors to the subject if the frequency of CD14_{CTX} cells in the test population is lower than the average frequency of CD14_{CTX} cells in the one or more reference populations, thereby treating the cancer in the subject.

3. The method of claim 1 or 2, wherein the lower frequency of CD14_{CTX} cells leads to higher disease-free survival (DFS).

4. The method of claim 1 or 2, wherein the frequency of CD14_{CTX} cells is determined by sequencing PBMCs.

5. The method of any one of claims 1-4, wherein a cell surface marker of the CD14_{CTX} cells is selected from the group consisting of T cell immunoglobulin and mucin domain-containing protein 3 (Tim3), CD29 (integrin 1), CD63, and CD68.

6. The method of claim 5, wherein the cell surface marker is Tim3.

7. The method of claim 5, wherein the cell surface marker is CD29.

8. A method of selecting a subject amendable to treatment with one or more immune checkpoint inhibitors from one or more subjects having or suspected of having cancer, the method comprising:
(a) isolating a test population of peripheral blood mononuclear cells (PBMCs) from the subject and one or more reference populations of PBMCs from each subject of the one or more subjects, wherein each reference population is isolated independently from each subject of the one or more subjects;
(b) quantifying frequency of CD4_{SOCS3} cells in the test population;
(c) quantifying frequency of CD4_{SOCS3} cells in the each reference population independently, and averaging the frequency CD4_{SOCS3} cells in the one or more reference populations;
(d) comparing the frequency of CD4_{SOCS3} cells in the test population to the average frequency of CD4_{SOCS3} cells in the one or more reference populations; and
(e) selecting the subject as the subject amendable to the treatment if the frequency of CD4_{SOCS3} cells in the test population is lower than the average frequency of CD4_{SOCS3} cells in the one or more reference populations.

9. A method of treating cancer in a subject having or suspected of having the cancer with one or more immune checkpoint inhibitors, the method comprising:
(a) isolating a test population of PBMCs from the subject and one or more reference populations of PBMCs from one or more subjects having or suspected of having cancer, wherein each reference population is isolated independently from each subject of the one or more subjects;
(b) quantifying frequency of CD4_{SOCS3} cells in the test population;
(c) quantifying frequency of CD4_{SOCS3} cells in the each reference population independently, and averaging the frequency of CD4_{SOCS3} cells in the one or more reference populations;
(d) comparing the frequency of CD4_{SOCS3} cells in the test population to the average frequency of CD4_{SOCS3} in the one or more reference populations; and
(e) administering a therapeutically effective amount of the one or more immune checkpoint inhibitors to the subject if the frequency of CD4_{SOCS3} cells in the test population is lower than the average frequency of CD4_{SOCS3} cells in the one or more reference populations, thereby treating the cancer in the subject.

10. The method of claim 8 or 9, wherein the frequency of CD4_{SOCS3} cells is correlated with the frequency of CD14_{CTX} cells of any one of claims 1-4.

11. The method of any one of claims 8-10, wherein the lower frequency of CD4_{SOCS3} cells leads to higher disease-free survival (DFS).

12. The method of any one of claims 8-11, wherein the frequency of CD4_{SOCS3} cells is determined by sequencing PBMCs.

13. The method of claim 4 or 12, wherein the sequencing method is single cell RNA sequencing (scRNAseq), single cell cellular indexing of transcriptomes or epitopes by sequencing (CITE-seq).

14. The method of any one of claims 1-13, wherein the one or more immune checkpoint inhibitors targets PD-1/PD-L1 pathway.

15. The method of claim 14, wherein the one or more immune checkpoint inhibitors targeting PD-1/PD-L1 pathway is selected from the group consisting of AMP-224, and AMP-514 (MEDI-0680), atezolizumab (TECENTRIQ®), avelumab (BAVENCIO®), BI-754091, budigalimab (ABBV-181), camrelizumab (SHR-1210), cemiplimab (LIBTAYO®), cosibelimab (CK-301), dostarlimab (Jemperli), durvalumab (IMFINZI®), INCMGA00012 (MGA012), JTX-4014, nivolumab (OPDIVO®), pembrolizumab (KEYTRUDA®), pidilizumab (CT-011), retifanlimab (MGA012), sasanlimab (PF-06801591), sintilimab (IBI308), spartalizumab (PDR001), tislelizumab (BGB-A317), toripalimab (JS 001), and zimberelimab (AB122).

16. The method of claim 15, wherein the one or more immune checkpoint inhibitors is pembrolizumab (Keytruda®).

17. The method of any one of claims 1-16, wherein the treatment comprises one or more therapeutic agents.

18. The method of claim 17, wherein the one or more therapeutic agents is GM-CSF.

19. The method of any one of claims 1-18, wherein the PBMCs are isolated before the treatment is administered.

20. The method of any one of claims 1-18, wherein the PBMCs are isolated after at least one cycle of the treatment is administered.

21. The method of claim 20, wherein the PBMCs are isolated at least one week, at least two weeks, at least 3 weeks, at least 4 weeks, at least 5 weeks, or at least 6 weeks after the treatment is administered.

22. The method of claim 21, wherein the PBMCs are isolated at least three weeks after the treatment is administered.

23. The method of any one of claims 1-22, further comprising:
(f) isolating a test group of intratumoral myeloid cells from the subject and one or more reference groups of intratumoral myeloid cells from the one or more subjects, wherein each reference group is isolated independently from each subject of the one or more subjects;
(g) quantifying frequency of Mac_{SPP1} cells in the test group;
(h) quantifying frequency of Mac_{SPP1} cells in the each reference group independently, and averaging the frequency of Mac_{SPP1} cells in the one or more reference groups; and
(i) comparing the frequency of Mac_{SPP1} cells from the test group to the average frequency of Mac_{SPP1} cells from the one or more reference groups.

24. The method of claim 23, wherein the frequency of Mac_{SPP1} cells in the test group is lower than the average frequency of Mac_{SPP1} cells in the one or more reference groups.

25. A method of selecting a subject amendable to treatment with one or more immune checkpoint inhibitors from one or more subjects having or suspected of having cancer, the method comprising:
(a) isolating a test group of intratumoral myeloid cells from the subject and one or more reference groups of intratumoral myeloid cells from the one or more subjects, wherein each reference group is isolated independently from each subject of the one or more subjects;
(b) quantifying frequency of Mac_{SPP1} cells in the test group;
(c) quantifying frequency of Mac_{SPP1} cells in the each reference group independently, and averaging the frequency of Mac_{SPP1} cells in the one or more reference groups;
(d) comparing the frequency of Mac_{SPP1} cells from the test group to the average frequency of Mac_{SPP1} cells from the one or more reference groups; and
(e) selecting the subject as the subject amendable to the treatment if the frequency of Mac_{SPP1} cells in the test group is lower than the average frequency of Mac_{SPP1} cells in the one or more reference groups.

26. A method of treating cancer in a subject having or suspected of having the cancer with one or more immune checkpoint inhibitors, the method comprising:
(a) isolating a test group of intratumoral myeloid cells from the subject and one or more reference groups of intratumoral myeloid cells from the one or more subjects, wherein each reference group is isolated independently from each subject of the one or more subjects;
(b) quantifying frequency of Mac_{SPP1} cells in the test group;
(c) quantifying frequency of Mac_{SPP1} cells in the each reference group independently, and averaging the frequency of Mac_{SPP1} cells in the one or more reference groups;
(d) comparing the frequency of Mac_{SPP1} cells from the test group to the average frequency of Mac_{SPP1} cells from the one or more reference groups; and
(e) administering a therapeutically effective amount of the one or more immune checkpoint inhibitors to the subject if the frequency of Mac_{SPP1} cells from the test group is lower than the average frequency of Mac_{SPP1} cells from the one or more reference groups, thereby treating the cancer in the subject.

27. The method of any one of claims 1-26, wherein the cancer is selected from the group consisting of biliary tract cancer, prostate cancer, colon cancer, kidney cancer, and skin cancer.
